SEQUENCE LISTING (37 sequences)

SEQ ID NO: 1 - DNA, 1080 bp, Homo sapiens
  source 1..1080: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaac

SEQ ID NO: 2 - protein, 360 aa, Homo sapiens
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 3 - DNA, 951 bp, Homo sapiens
  source 1..951: mol_type = other DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 4 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 5 - DNA, 1410 bp, Homo sapiens
  source 1..1410: mol_type = other DNA
atggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 6 - protein, 470 aa, Homo sapiens
MAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 7 - DNA, 376 bp, synthetic construct
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 8 - DNA, 57 bp, Teschovirus A
  source 1..57: mol_type = other DNA
gccacgaacttctctctgttaaagcaagcaggagatgttgaagaaaaccccgggcct

SEQ ID NO: 9 - protein, 19 aa, Teschovirus A
ATNFSLLKQAGDVEENPGP

SEQ ID NO: 10 - DNA, 54 bp, Thosea asigna virus
  source 1..54: mol_type = other DNA
gagggcaggggaagtcttctaacatgcggggacgtggaggaaaatcccggccca

SEQ ID NO: 11 - protein, 18 aa, Thosea asigna virus
EGRGSLLTCGDVEENPGP

SEQ ID NO: 12 - DNA, 135 bp, synthetic construct
  source 1..135: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctggatc

SEQ ID NO: 13 - DNA, 4933 bp, synthetic construct
  source 1..4933: mol_type = other DNA
ctatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcatcccaatggcgcgccgagcttggctcgagcatggtcat

SEQ ID NO: 14 - DNA, 3594 bp, synthetic construct
  source 1..3594: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttt

SEQ ID NO: 15 - DNA, 7250 bp, synthetic construct
  source 1..7250: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 16 - DNA, 141 bp, synthetic construct
  source 1..141: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 18 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 19 - DNA, 747 bp, synthetic construct
  source 1..747: mol_type = other DNA
atgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaa

SEQ ID NO: 20 - protein, 248 aa, synthetic construct
MSRLDKSKIINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 21 - DNA, 5435 bp, synthetic construct
  source 1..5435: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 22 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 23 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 24 - DNA, 204 bp, synthetic construct; identical to SEQ ID NO: 17
  source 1..204: mol_type = other DNA

SEQ ID NO: 25 - DNA, 380 bp, synthetic construct; identical to SEQ ID NO: 18
  source 1..380: mol_type = other DNA

SEQ ID NO: 26 - DNA, 747 bp, synthetic construct
  source 1..747: mol_type = other DNA
atgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaa

SEQ ID NO: 27 - protein, 248 aa, synthetic construct
MSRLDKSKVINSALELLNGVGIEGLTTRKLAQKLGVEQPTLYWHVKNKRALLDALPIEMLDRHHTHSCPLEVESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 28 - DNA, 5435 bp, synthetic construct
  source 1..5435: mol_type = other DNA
tcgcgcgtttcggtgatgacggtgaaaacctctgacacatgcagctcccggagactgtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatactatggttgctttgacgtatgcggtgtgaaataccgcacagatgcgtaaggagaaaataccgcatcaggcgcccctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

SEQ ID NO: 29 - DNA, 141 bp, synthetic construct; identical to SEQ ID NO: 22
  source 1..141: mol_type = other DNA

SEQ ID NO: 30 - DNA, 122 bp, synthetic construct
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 31 - DNA, 589 bp, synthetic construct; identical to SEQ ID NO: 23
  source 1..589: mol_type = other DNA

SEQ ID NO: 32 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 33 - DNA, 141 bp, synthetic construct; identical to SEQ ID NO: 32
  source 1..141: mol_type = other DNA

SEQ ID NO: 34 - DNA, 141 bp, synthetic construct; identical to SEQ ID NO: 32
  source 1..141: mol_type = other DNA

SEQ ID NO: 35 - DNA, 4460 bp, synthetic construct
  source 1..4460: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattctttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagcggccgcgccaccatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaacgctagcggcagcggcgccacgaacttctctctgttaaagcaagcaggagatgttgaagaaaaccccgggcctgcatgcatgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatggcatgcggctccggcgagggcaggggaagtcttctaacatgcggggacgtggaggaaaatcccggcccactcgagatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatgactagtgcgcgcagcggccgaccatggcccaacttgtttattgcagcttataatggttacaaataaagcaatagcatcacaaatttcacaaataaagcatttttttcactgcattctagttgtggtttgtccaaactcatcaatgtatcttatcatgtctggatctcggtaccggatccaaattcccgataaggatcttcctagagcatggctacgtagataagtagcatggcgggttaatcattaactacaaggaaccccta

SEQ ID NO: 36 - DNA, 2735 bp, synthetic construct
  source 1..2735: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtctagactggacaagagcaaaatcataaacagcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacagctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaagcaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 37 - DNA, 4946 bp, synthetic construct
  source 1..4946: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctcggtccgcacgatctcaattcggccattacggccggatccggctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacgctaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccccgaattcaccatgtcccgcttggataagagcaaggtaataaatagcgcactcgaactcctcaacggcgtgggcatcgaaggtctgactactcgaaagctcgcccagaaattgggtgtggagcaacctacattgtattggcatgtcaagaacaaaagagccctgctggacgctcttcctattgaaatgcttgacaggcatcacactcattcctgcccccttgaggtcgagagttggcaagattttctccgaaacaatgcaaagtcctaccgctgcgcacttttgtcccatagggatggagcaaaagtgcacctgggaaccaggccaacagagaaacaatacgagactctcgagaaccagttggctttcttgtgccaacaggggttctcacttgaaaatgccctttacgcactgtcagccgttggacattttaccctggggtgcgttcttgaggagcaagaacatcaggttgctaaggaggagcgcgagactccaaccactgattctatgccacctttgctgaaacaggccattgaacttttcgatagacagggtgctgaacctgcctttctcttcgggttggagctgattatttgtggtctcgaaaaacagctgaaatgtgaaagtggtggccctactgacgccctcgatgatttcgacctggatatgctgccagccgatgcacttgatgatttcgatttggatatgcttccagccgacgcactggacgacttcgatttggacatgcttcccggttaactaagtaaggatcatcttaattaaatcgataaggatctggccgcctcggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaactagcgcgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctgttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaaacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggcccttttgtc

(19) United States
(12) Patent Application Publication
(10) Pub. No.: US 2026/0108630 A1
(43) Pub. Date: Apr. 23, 2026

(54) PARTIAL EPIGENETIC REPROGRAMMING FOR LIVER DISEASE TREATMENT

(71) Applicant: Life Biosciences, Inc., Boston, MA (US)

(72) Inventors: Sharon Rosenzweig-Lipson, Brunswick, NJ (US); Ming Yang, Baltimore, MD (US); Richard Davis, Albany, NY (US); Michel Wathier, Milton, MA (US)

(21) Appl. No.: 19/360,504

(22) Filed: Oct. 16, 2025

Related U.S. Application Data
(60) Provisional application No. 63/708,587, filed on Oct. 17, 2024.
(60) Provisional application No. 63/867,895, filed on Aug. 21, 2025.
(60) Provisional application No. 63/877,679, filed on Sep. 8, 2025.

(51) Int. Cl. A61K 48/00; A61K 38/17; A61P 1/16

(52) U.S. Cl. CPC A61K 48/005; A61K 38/1709; A61K 48/0075; A61P 1/16

(57) ABSTRACT

Provided are methods for treating a subject with a liver disease such as metabolic dysfunction-associated steatotic liver disease (MASLD) or metabolic dysfunction-associated steatohepatitis (MASH). The method may include administering to the subject one or more nucleic acids encoding OCT4, SOX2 and KLF4. The administration may rejuvenate hepatocytes or liver stellate cells in the subject, and may improve a symptom of the liver disease.

### CROSS-REFERENCE TO RELATED APPLICATIONS

This application claims the benefit of U.S. Provisional Application No. 63/708,587, filed on Oct. 17, 2024, U.S. Provisional Application No. 63/867,895, filed on Aug. 21, 2025, and U.S. Provisional Application No. 63/877,679, filed on Sep. 8, 2025, the content of each of which is hereby incorporated by reference in its entirety.

### INCORPORATION BY REFERENCE OF SEQUENCE LISTING

A Sequence Listing in the form of an XML file (entitled “LBO-00501-SL”, created on Aug. 21, 2025, and having a size of 81,963 bytes) is hereby incorporated by reference in its entirety.

### BACKGROUND

Liver diseases such as metabolic dysfunction-associated steatotic liver disease (MASLD; formerly called non-alcoholic fatty liver disease (NAFLD)) and metabolic dysfunction-associated steatohepatitis (MASH; formerly called non-alcoholic steatohepatitis (NASH)) are common. Improved treatments are needed.

### SUMMARY

Provided are methods of treating a subject having a liver disease. The treatment may include delivery of octamer-binding transcription factor 4 (OCT4), sex determining region Y-box 2 (SOX2) and Krüppel-like factor 4 (KLF4) for partial epigenetic reprogramming. Partial reprogramming in liver cells may be achieved by delivery of Yamanaka factors excluding MYC proto-oncogene (c-Myc). This may rejuvenate the liver cells, and improve a symptom or aspect of the disease. The rejuvenation may include a reversal of epigenetic change. Rejuvenation may also include reverting the liver disease to an earlier stage of the disease.

Provided herein, in some embodiments, are methods of treating a subject having a liver disease, reversing epigenetic change in a subject having a liver disease, or reverting a liver disease to an earlier stage of disease in a subject, comprising: administering to the subject an effective amount of a pharmaceutical composition comprising one or more polynucleotides encoding OCT4, SOX2 and KLF4, and not encoding c-Myc. In some embodiments, reversing epigenetic change comprises reverting a marker of epigenetic change associated with the liver disease to a state associated with a healthy liver or to a state associated with a stage of the disease that is earlier than a stage at which the pharmaceutical composition is administered. In some embodiments, the marker of epigenetic change comprises DNA methylation. In some embodiments, the administration rejuvenates liver cells in the subject. In some embodiments, the liver cells comprise hepatocytes or stellate cells. In some embodiments, the administration improves a symptom of the liver disease in the subject. In some embodiments, the composition does not reprogram a cell, tissue, or organ to a pluripotent state in the subject. In some embodiments, the composition does not induce c-Myc expression in the subject. In some embodiments, the one or more polynucleotides comprise DNA. In some embodiments, the one or more polynucleotides comprise an expression vector. In some embodiments, the one or more polynucleotides comprise a plasmid. In some embodiments, the one or more polynucleotides comprise RNA. In some embodiments, the RNA comprises messenger RNA (mRNA). In some embodiments, the OCT4, SOX2 and KLF4 are operably linked to one or more promoters. In some embodiments, the one or more promoters comprise an inducible promoter. Some embodiments include administering to the subject an inducing agent to induce expression of OCT4, SOX2, and KLF4 in the subject. In some embodiments, the one or more promoters comprise a constitutively active promoter. In some embodiments, the expression vector is polycistronic for OCT4, SOX2 and KLF4. In some embodiments, the expression vector further comprises a polynucleotide encoding a self-cleaving peptide. In some embodiments, the polynucleotide comprises inverted terminal repeats (ITRs). In some embodiments, the expression vector is a viral expression vector, wherein the viral vector is a lentivirus, a retrovirus, an adenovirus, alphavirus, vaccinia virus, or an adeno-associated virus (AAV) vector, optionally where the expression vector is an AAV1, AAV2, AAV3, AAV4, AAV5, AAV6, AAV7, AAV8, AAV9, AAV 10, AAV11, or AAVDJ vector, optionally where the expression vector is an AAV6 or AAV8 vector. In some embodiments, the composition comprises a lipid nanoparticle (LNP). In some embodiments, the LNP comprises a cationic lipid, an ionizable lipid, a helper lipid, a polyethylene glycol (PEG) conjugated (PEGylated) lipid, a cholesterol-based lipid, or a combination thereof. In some embodiments, the LNP comprises a targeting moiety. In some embodiments, the targeting moiety comprises N-acetylgalactosamine (GalNAc), an antibody, an antibody fragment, an scFv, a retinoid, ApoE, or analog thereof. In some embodiments, the polynucleotide comprises an OCT4 sequence at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 1. In some embodiments, the OCT4 comprises an amino acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 2. In some embodiments, the polynucleotide comprises a SOX2 nucleic acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 3. In some embodiments, the SOX2 comprises an amino acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 4. In some embodiments, the polynucleotide comprises a KLF4 nucleic acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 5. In some embodiments, the KLF4 comprises an amino acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 6. In some embodiments, the administration is intravenous. In some embodiments, the administration comprises an injection. Some embodiments include identifying the subject as having the liver disease before administering the pharmaceutical composition to the subject. In some embodiments, the liver disease is chronic. In some embodiments, the liver disease comprises liver steatosis. In some embodiments, the liver disease comprises metabolic dysfunction-associated steatotic liver disease (MASLD) or metabolic dysfunction-associated steatohepatitis (MASH). In some embodiments, the liver disease comprises liver fibrosis. In some embodiments, the method decreases plasma ALT in the subject. In some embodiments, the method decreases plasma AST in the subject. In some embodiments, the method decreases plasma total bile acids in the subject. In some embodiments, the method decreases plasma total cholesterol in the subject. In some embodiments, the method decreases the percentage of hepatocytes with lipid droplets in the subject. In some embodiments, the method decreases liver weight in the subject. In some embodiments, the method improves NAFLD score of the subject by at least 1 point. In some embodiments, the method improves steatosis score of the subject by at least 1 point. In some embodiments, the method does not cause a decrease in body weight of the subject and/or body weight of the subject does not decrease (e.g., does not decrease during and/or as a result of treatment). In some embodiments, the liver disease comprises cirrhosis. In some embodiments, the subject is a human.

Provided herein, in some embodiments, are methods for treating a subject in need of treatment for MASLD or MASH, reversing epigenetic change in a subject in need of treatment for MASLD or MASH, or reverting MASLD or MASH to an earlier stage of disease in a subject in need thereof, the method comprising administering to the subject a pharmaceutically effective amount of a pharmaceutical composition comprising an expression vector encoding three transcription factors, wherein the three transcription factors consist of OCT4, SOX2, and KLF4. In some embodiments, reversing epigenetic change comprises reverting a marker of epigenetic change associated with MASLD or MASH to a state associated with a healthy liver or to a state associated with a stage of MASLD or MASH that is earlier than a MASLD or MASH stage at which the pharmaceutical composition is administered. In some embodiments, the marker of epigenetic change comprises DNA methylation.

In some embodiments including a method of treating a subject as set forth herein (e.g., with an OSK therapy), the subject is administered, the subject has been administered, and/or the method includes administering to the subject an obesity therapy. In some embodiments, the obesity therapy includes a GLP-1 agonist, optionally where the GLP-1 agonist includes semaglutide, liraglutide, dulaglutide, exenatide, tirzepatide, lixisenatide, or albiglutide. In some embodiments, the subject is administered, the subject has been administered, and/or the method includes administering to the subject glucagon and/or a glucose-dependent insulinotropic polypeptide. In some embodiments, the obesity therapy includes orlistat, phentermine, topiramate, phentermine-topiramate, naltrexone, bupropion, naltrexone-bupropion, or setmelanotide. In some embodiments, the obesity therapy includes a surgery or device.

In some embodiments including a method of treating a subject as set forth herein (e.g., with an OSK therapy), the subject is administered, the subject has been administered, and/or the method includes administering to the subject a liver disease and/or MASH therapy. In some embodiments, the liver disease and/or MASH therapy includes a THR-agonist. In some embodiments, the liver disease and/or MASH therapy includes resmetirom. In some embodiments, the liver disease and/or MASH therapy includes a PPAR agonist. In some embodiments, the PPAR agonist includes lanifibranor or saroglitazar. In some embodiments, the liver disease and/or MASH therapy includes an FGF21 analog. In some embodiments, the FGF21 analog includes efruxifermin or pegbelfermin. In some embodiments, the liver disease and/or MASH therapy includes vitamin E.

Provided herein, in some embodiments, are methods of treating a subject having a liver disease and in need of treatment thereof or at risk of liver disease and in need of treatment thereof, comprising administering to the subject an amount of a pharmaceutical composition comprising one or more polynucleotides encoding: octamer-binding transcription factor 4 (OCT4), sex determining region Y-box 2 (SOX2), and Krüppel-like factor 4 (KLF4); wherein the one or more polynucleotides do not encode MYC proto-oncogene (c-Myc). In some embodiments, the liver disease is a chronic liver disease. In some embodiments, the liver disease is liver steatosis. In some embodiments, the liver disease is liver fibrosis. In some embodiments, the liver disease is cirrhosis. In some embodiments, the subject has or is suspected of having metabolic dysfunction-associated steatotic liver disease. In some embodiments, the subject has or is suspected of having metabolic dysfunction-associated steatohepatitis. In some embodiments, a liver biopsy from the subject after the administration exhibits an improvement in one or more epigenetic markers, as compared to a baseline liver biopsy from the subject. In some embodiments, the liver biopsy is of one or more hepatocyte cells. In some embodiments, the liver biopsy is of one or more stellate cells. In some embodiments, the administration results in a biochemical response in the subject. In some embodiments, the administration results in an improvement in one or more biomarkers of the liver disease in the subject. In some embodiments, the administration results in an improvement in one or more symptoms of the subject's liver disease, as compared to baseline. In some embodiments, the administration results in an improvement in the stage of the subject's liver disease. In some embodiments, the pharmaceutical composition is administered intravenously. In some embodiments, the pharmaceutical composition is administered subcutaneously or intramuscularly. In some embodiments, the administration of the pharmaceutical composition does not induce c-Myc expression in the subject. In some embodiments, the pharmaceutical composition comprises a plasmid that comprises the one or more polynucleotides. In some embodiments, the pharmaceutical composition comprises an expression vector that comprises the one or more polynucleotides. In some embodiments, the expression vector is a viral expression vector, for example, a lentivirus, a retrovirus, an adenovirus, alphavirus, vaccinia virus, or an adeno-associated virus (AAV) vector. In some embodiments, the expression vector is polycistronic for OCT4, SOX2, and KLF4. In some embodiments, the one or more polynucleotides are in the form of deoxyribonucleic acid. In some embodiments, the one or more polynucleotides are in the form of ribonucleic acid (RNA). In some embodiments, the RNA comprises mRNA. In some embodiments, the pharmaceutical composition comprises one or more lipid nanoparticles that comprise the one or more polynucleotides. In some embodiments, the OCT4, SOX4, and/or KLF4 are each operably linked to one or more promoters. In some embodiments, at least one of the promoters is an inducible promoter. In some embodiments, at least one of the promoters is a constitutively active promoter. In some embodiments, at least one of the polynucleotides comprises an OCT4 sequence at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 1. In some embodiments, at least one of the polynucleotides comprises an OCT4 sequence at least 95% identical, at least 96% identical, at least 97% identical, at least 98% identical, at least 99% identical, or 100% identical, to SEQ ID NO: 1. In some embodiments, at least one of the polynucleotides comprises a SOX2 sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 3. In some embodiments, at least one of the polynucleotides comprises a SOX2 sequence having at least 95% identical, at least 96% identical, at least 97% identical, at least 98% identical, at least 99% identical, or 100% identical, to SEQ ID NO: 3. In some embodiments, at least one of the polynucleotides comprises a KLF4 sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 5. In some embodiments, at least one of the polynucleotides comprises a KLF4 sequence having at least 95% identical, at least 96% identical, at least 97% identical, at least 98% identical, at least 99% identical, or 100% identical, to SEQ ID NO: 5.

### BRIEF DESCRIPTION OF THE DRAWINGS

FIG. 1 is an illustrative vector map of TRE3G-OSK-SV40 pA, an AAV2 vector encoding OSK (SEQ ID NO: 15).

FIG. 2 includes a table showing some aspects relating to a NAFLD activity score. The definitions in this figure are applicable only within the context of the figure.

FIG. 3 includes a table showing some aspects relating to MASH fibrosis score. The definitions in this figure are applicable only within the context of the figure.

FIG. 4 graphically depicts Sox2 and Klf4 protein measurements in response to administration of lipid nanoparticles that included mRNA encoding OSK. The figure includes data from the following in order from left to right above each protein name: LNP #1, LNP #2, LNP #3, LNP #4, and vehicles.

FIG. 5A includes plots showing liver inflammation and liver fibrosis in livers of mice affected by CCl₄. Histology scores in the figures show significant inflammation and fibrosis induced by CCl₄.

FIG. 5B is a DMC heat map showing DNA methylation changes in livers of mice affected by CCl₄.

FIG. 6 is a schematic representation of an experiment demonstrating use of AAV-OSK for treatment of MASH in a mouse model.

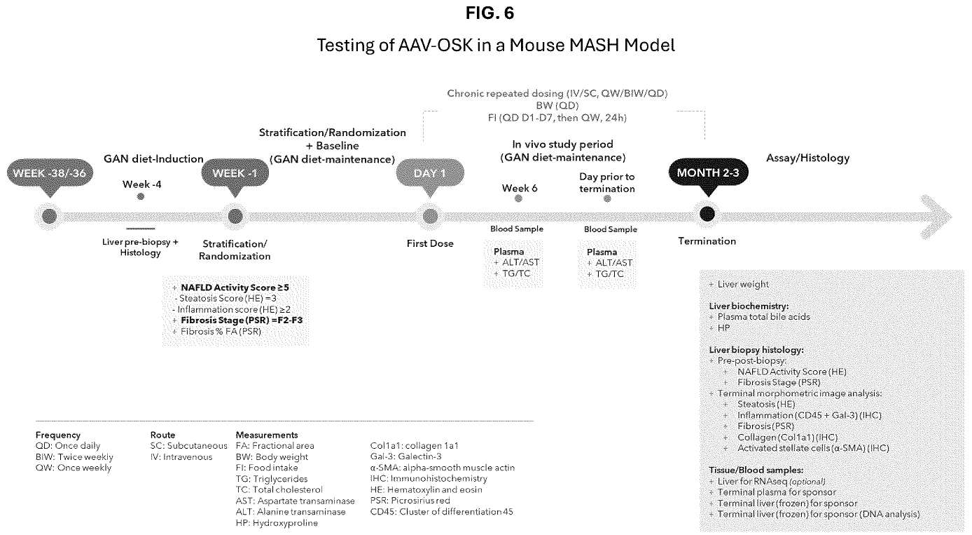

FIG. 7 is a graph showing the effect of AAV-OSK on body weight in a mouse model of MASH. AAV-OSK therapy did not lower body weight. The left bar of the graph represents a vehicle control, and the right bar of the graph represents OSK therapy.

FIG. 8 is a graph showing the effect of AAV-OSK on alanine transaminase (ALT) in plasma in a mouse model of MASH. AAV-OSK therapy significantly reduced ALT. The left bar of the graph represents a vehicle control, and the right bar of the graph represents OSK therapy.

FIG. 9 is a graph showing the effect of AAV-OSK on aspartate transaminase (AST) in plasma in a mouse model of MASH. AAV-OSK therapy significantly reduced AST. The left bar of the graph represents a vehicle control, and the right bar of the graph represents OSK therapy.

FIG. 10 is a graph showing the effect of AAV-OSK on total bile acids in plasma in a mouse model of MASH. AAV-OSK therapy significantly reduced total bile acids. The left bar of the graph represents a vehicle control, and the right bar of the graph represents OSK therapy.

FIG. 11 is a graph showing the effect of AAV-OSK on total cholesterol in plasma in a mouse model of MASH. AAV-OSK therapy significantly reduced total cholesterol. The left bar of the graph represents a vehicle control, and the right bar of the graph represents OSK therapy.

FIG. 12 is a graph showing the effect of AAV-OSK on nonalcoholic fatty liver disease (NAFLD) score in a mouse model of MASH. AAV-OSK therapy significantly improved NAFLD score. The left bar of the graph represents a vehicle control, and the right bar of the graph represents OSK therapy.

FIG. 13 is a graph showing the effect of AAV-OSK on the percentage of hepatocytes with lipid droplets in a mouse model of MASH. AAV-OSK therapy significantly reduced the percentage of hepatocytes with lipid droplets. The left bar of the graph represents a vehicle control, and the right bar of the graph represents OSK therapy.

FIG. 14 is a graph showing the effect of AAV-OSK on liver weight in a mouse model of MASH. AAV-OSK therapy significantly decreased liver weight. The left bar of the graph represents a vehicle control, and the right bar of the graph represents OSK therapy.

FIG. 15 includes plot showing the effect of AAV-OSK on steatosis score in a mouse model of MASH. AAV-OSK therapy significantly improved steatosis score. The left bar of the graph represents a vehicle control, and the right bar of the graph represents OSK therapy.

FIG. 16 is a graph showing the concentration of KLF4 protein (ng/mg total protein) in liver tissue in a mouse model of MASH, with or without administration of OSK therapy. OSK therapy significantly increased the level of KLF4 protein in liver.

FIG. 17 is a graph showing the concentration of SOX2 protein (ng/mg total protein) in liver tissue in a mouse model of MASH, with or without administration of OSK therapy. OSK therapy significantly increased the level of SOX2 protein in liver.

### DETAILED DESCRIPTION

Octamer-binding transcription factor 4 (OCT4), sex determining region Y-box 2 (SOX2) and Krüppel-like factor 4 (KLF4) (together, “OSK”) partial epigenetic reprogramming may address an unmet need for improved liver disease treatment, and be useful in treating liver diseases. OSK therapy may reverse the advancement of the DNA methylation clock, improve outcomes in aging, extend lifespan, and result in better disease outcomes. Transient OCT4, SOX2, KLF4, and MYC proto-oncogene (c-Myc) (together, “OSKM”) expression may induce cellular plasticity and counteract liver failure, or reduce fibrosis. OSK therapy may be efficacious at reversing the DNA methylation clock and other epigenetic markers, and for improving the symptoms of metabolic dysfunction-associated steatotic liver disease (MASLD) or metabolic dysfunction-associated steatohepatitis (MASH).

OSK therapy may be efficacious in liver diseases because epigenetic changes, such as changes in DNA methylation, may occur in MASH/MASLD. Similarly, epigenetic changes may occur in animal models of liver disease such as the Gubra Amylin-diet induced obesity MASH model and the CCL4-liver toxicity model. As such, these models may be tested to demonstrate an improvement in a liver disease by OSK therapy as provided herein.

Disclosed herein, in some embodiments, are methods for treating a subject having a liver disease, reversing epigenetic change in a subject having a liver disease, or reverting a liver disease to an earlier stage of disease in a subject. The method may include treating a subject having a liver disease. The method may include reversing epigenetic change in a subject having a liver disease. The method may include reverting a liver disease to an earlier stage of disease in a subject. The method may include administering a composition herein.

Provided herein, in some embodiments, are methods for treating a subject in need of treatment for MASLD or MASH, reversing epigenetic change in a subject in need of treatment for MASLD or MASH, or reverting MASLD or MASH to an earlier stage of disease in a subject in need thereof. The method may include treating a subject in need of treatment for MASLD or MASH. The method may include reversing epigenetic change in a subject in need of treatment for MASLD or MASH. The method may include reverting MASLD or MASH to an earlier stage of disease in a subject in need thereof. The method may include administering a composition herein.

Disclosed herein, in some embodiments, are methods for treating a subject with a liver disease such as MASLD or MASH that include administration of one or more nucleic acids encoding OCT4, SOX2 and KLF4. The one or more nucleic acids may exclude c-Myc, which may avoid fully reprogramming cells. The administration may be useful for treatment of liver disease by rejuvenating liver cells or may improve a liver disease symptom or revert the liver disease to an earlier stage. The administration may also be useful in preventing liver disease when administered to an at risk population (e.g. obese).

### Compositions

Disclosed herein, in some embodiments, are compositions. The composition may include a pharmaceutical composition. The pharmaceutical composition may include a pharmaceutically acceptable carrier. The composition may include a viral delivery vehicle (e.g. AAV) or a nanoparticle (e.g. a lipid nanoparticle). The composition may include one or more polynucleotides. In some embodiments, the composition is used in a method described herein, such as a method of treating a subject with a liver disease. The treatment may include rejuvenating cells. In some embodiments, the composition is used in a method that includes rejuvenating cells of a subject with a liver disease. The rejuvenation may include reversal of epigenetic change. Epigenetic change may include a change in methylation or other chromatin changes that may occur leading up to, after, or during a disease, and may be causative of at least some aspects of the disease. In some embodiments, epigenetic change is associated with liver disease and modifying the epigenome to a pre-disease state can result in treatment and/or prevention of disease. In some embodiments, the composition is used in a method that includes reversing epigenetic change in a subject with a liver disease. The composition may include an expression vector comprising a promoter operably linked to the partial reprogramming factors.

Disclosed herein, in some embodiments, are one or more polynucleotides. The one or more polynucleotides may be or include a polynucleotide or nucleic acid. The one or more polynucleotides may be or include multiple polynucleotides or nucleic acids. The terms, “polynucleotide” and “nucleic acid” may be used interchangeably. The one or more polynucleotides may encode partial reprogramming factors. The one or more polynucleotides may include an expression vector comprising a promoter operably linked to the partial reprogramming factors. The one or more polynucleotides may be recombinant. The one or more polynucleotides may be synthetic.

The present disclosure provides nucleic acid molecules that include a nucleic acid sequence encoding OCT4, a nucleic acid sequence encoding SOX2, a nucleic acid sequence encoding KLF4, or any combination thereof, and in the absence of an exogenous nucleic acid sequence encoding c-Myc. The nucleic acid molecule may be a vector, including for example an expression vector. In certain embodiments, the nucleic acid molecule includes a nucleic acid sequence encoding OCT4. In certain embodiments, the nucleic acid molecule includes a nucleic acid sequence encoding SOX2. In certain embodiments, the nucleic acid molecule includes a nucleic acid sequence encoding KLF4. In certain embodiments, the nucleic acid molecule includes any two of a nucleic acid sequence encoding OCT4, a nucleic acid sequence encoding SOX2, and a nucleic acid sequence encoding KLF4. In certain embodiments, the nucleic acid molecule includes a first nucleic acid sequence encoding OCT4, a second nucleic acid sequence encoding SOX2, and a third nucleic acid sequence encoding KLF4. In certain embodiments, OCT4, SOX2, KLF4, or any combination thereof is a human protein. In certain embodiments, OCT4, SOX2, KLF4, or any combination thereof is a non-human protein, for example, a protein from one or more mammals including from one or more primates (e.g., cynomolgus monkeys, rhesus monkeys). If two or more of OCT4, SOX2, and KLF4 are on one nucleic acid molecule, they may be in any order. The words “first.” “second,” and “third” do not necessarily imply an order of the genes on the nucleic acid molecule.

In some embodiments, the one or more polynucleotides comprise DNA. In some embodiments, the one or more polynucleotides comprise a plasmid. In some embodiments, the one or more polynucleotides comprise an expression vector. In some embodiments, the one or more polynucleotides comprise RNA. In some embodiments, the RNA comprises messenger RNA (mRNA).

### Partial Reprogramming Factors

Disclosed herein, in some embodiments, are reprogramming factors such as some Yamanaka factors. Yamanaka factors octamer-binding transcription factor 4 (OCT4), sex determining region Y-box 2 (SOX2), Krüppel-like factor 4 (KLF4), and MYC proto-oncogene (c-Myc). The reprogramming factors may be useful for epigenetic reprogramming. In some embodiments, reprogramming factors include or consist of partial reprogramming factors. The partial reprogramming factors may be useful for partial epigenetic reprogramming, which may refresh cells such as target cells without inducing pluripotency in the cells. Partial reprogramming factors may be or include a subset of OCT4, SOX2, KLF4, and c-Myc (e.g. a subset of 2 or 3 of the Yamanaka factors). Partial reprogramming factors may exclude c-Myc. Partial reprogramming factors may exclude a functional variant of c-Myc. Partial reprogramming factors may be or include OCT4, SOX2, KLF4 (e.g. without c-Myc). Partial reprogramming factors may be or include one or more variants of OCT4, SOX2, or KLF4. The reprogramming factors or partial reprogramming factors may be encoded by one or more polynucleotides or nucleic acids. In some embodiments, the OCT4, SOX2 and KLF4 are encoded by an expression vector or nucleic acid. In some embodiments, the OCT4, SOX2 and KLF4 are encoded by multiple expression vectors or nucleic acids.

In some embodiments, the polynucleotide comprises an OCT4 sequence at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 1. In certain embodiments, the nucleic acid sequence encoding OCT4 is at least 70% (e.g., at least 75%, 80%, 85%, 90%, 95%, 98%, 99%, or 100%) identical to SEQ ID NO: 1. In some embodiments, the OCT4 comprises an amino acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 2. In certain embodiments, OCT4 comprises an amino acid sequence at least 70% (e.g., at least 75%, 80%, 85%, 90%, 95%. 98%, 99%, or 100%) identical to SEQ ID NO: 2.

In some embodiments, the polynucleotide comprises a SOX2 nucleic acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 3. In certain embodiments, the nucleic acid sequence encoding SOX2 is at least 70% (e.g., at least 75%, 80%, 85%, 90%, 95%, 98%, 99%, or 100%) identical to SEQ ID NO: 3. In some embodiments, the SOX2 comprises an amino acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 4. In certain embodiments, SOX2 comprises an amino acid sequence at least 70% (e.g., at least 75%, 80%, 85%, 90%, 95%, 98%, 99%, or 100%) identical to SEQ ID NO: 4.

In some embodiments, the polynucleotide comprises a KLF4 nucleic acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 5. In certain embodiments, the nucleic acid sequence encoding KLF4 is at least 70% identical (e.g., at least 75%, 80%, 85%, 90%, 95%, 98%, 99%, or 100%) to SEQ ID NO: 5.

In some embodiments, the KLF4 comprises an amino acid sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 6. In certain embodiments, KLF4 comprises an amino acid sequence at least 70% (e.g., at least 75%, 80%, 85%, 90%, 95%, 98%, 99%, or 100%) identical to SEQ ID NO: 6.

### Expression Vectors

Disclosed herein, in some embodiments, are expression vectors. Expression vectors may include one or more polynucleotides. An expression vector may include a polynucleotide.

One or more expression vectors may encode and express reprogramming factors. One or more expression vectors may express partial reprogramming factors such as OCT4, SOX2, or KLF4. One or more expression vectors may express OCT4, SOX2, and KLF4. In some embodiments, the expression vectors include an expression vector that encodes and expresses OCT4, SOX2 and KLF4. In some embodiments, the expression vectors include multiple expression vectors that encode and expresses OCT4, SOX2 and KLF4.

The expression vector may be or include a nucleic acid. The expression vector may include a nucleic acid. The expression vector may be a nucleic acid. The nucleic acid may be or include DNA. The nucleic acid may be or include RNA. The expression vector may include a promoter. In some embodiments, the expression vector is polycistronic for OCT4, SOX2 and KLF4. In some embodiments, the expression vector further comprises a polynucleotide encoding a self-cleaving peptide. In some embodiments, the polynucleotide comprises inverted terminal repeats (ITRs).

The expression vector may include or be included with a delivery vehicle. The expression vector may include a delivery vehicle such as a viral delivery vehicle or a lipid nanoparticle. The delivery vehicle may include a virus or aspect of a virus. The delivery vehicle may include a lipid or lipids. The delivery vehicle may include a nanoparticle. The delivery vehicle may include a lipid nanoparticle.

The expression vector may be in a cell. The expression vector may be expressed in a cell. The cell may be eukaryotic. The cell may be a vertebrate cell. The cell may be an animal cell. The cell may be a mammalian cell. The cell may be a human cell. The cell may be a cell of a subject herein, such as a subject administered a composition such as an expression vector herein. The cell may be in vivo. The cell may be cultured. The cell may be a cell of a cell line. The cell may be in vitro.

In certain embodiments, the nucleic acid molecule (e.g., an expression vector encoding OCT4, KLF4, SOX2, an inducing agent, or a combination thereof) of the present disclosure may further comprise a nucleic acid sequence encoding a selection agent (e.g., an antibiotic, including blasticidin, geneticin, hygromycin B, mycophenolic acid, puromycin, zeocin, actinomycin D, ampicillin, carbenicillin, kanamycin, and neomycin) and/or detectable marker (e.g., GFP, RFP, luciferase, CFP, mCherry, DsRed2FP, mKate, biotin, FLAG-tag, HA-tag, His-tag, Myc-tag, V5-tag, etc.).

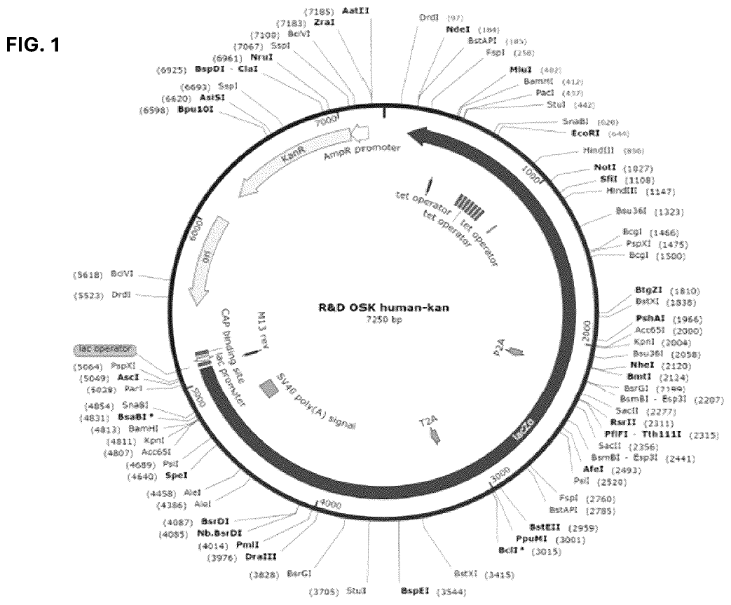

In some embodiments, the expression vector encoding OCT4, KLF4, and SOX2 comprises the sequence provided in SEQ ID NO: 13, SEQ ID NO: 14, or SEQ ID NO: 15. In some embodiments, the expression vector encoding OCT4, KLF4, and SOX2 comprises the elements depicted in FIG. 1, or a combination thereof. The expression vector may be a viral vector. The viral vector may be an adeno-associated virus (AAV) vector, retroviral vector, lentiviral vector, herpes viral vector, and the like.

### Promoters

Disclosed herein, in some embodiments, are promoters. The promoter may be included among one or more polynucleotides. Multiple promoters may be used when multiple polynucleotides are present. One or more promoters may drive expression of reprogramming factors. One or more promoters may drive expression of partial reprogramming factors such as OCT4, SOX2, or KLF4. One or more promoters may drive expression of OCT4, SOX2, and KLF4. In some embodiments, the OCT4, SOX2 and KLF4 are operably linked to one or more promoters. In some embodiments, the one or more promoters comprise an inducible promoter. In some embodiments, the one or more promoters comprise a constitutively active promoter. A promoter may include a liver cell-specific promoter or a liver tissue-specific promoter.

In some embodiments, the nucleic acid molecule of the present disclosure includes an inducible promoter. In some embodiments, the nucleic acid molecule has one inducible promoter. In such instances, the expression of OCT4, SOX2, and KLF4 are under the control of the same inducible promoter. In some embodiments, the nucleic acid molecule has more than one inducible promoter. The inducible promoter may include a tetracycline-responsive element (TRE) (e.g., a TRE3G promoter, a TRE2 promoter, or a P tight promoter), mifepristone-responsive promoters (e.g., GAL4-Elb promoter), or a coumermycin-responsive). As an example, a TRE (e.g., TRE3G) promoter may comprise a nucleic acid sequence that is at least 70% (e.g., at least 75%, 80%, 85%, 90%, 95%, 98%, 99%, or 100%) identical to SEQ ID NO: 7. See, e.g., US Publ. Appl. No. 2021-0403923 A, and the International Publ. No. WO2020/069339.

In certain embodiments, the inducing agent is capable of inducing expression of the first (e.g., OCT4), second (e.g., SOX2), third (e.g., KLF4) nucleic acids, or any combination thereof from the inducible promoter in the presence of a tetracycline (e.g., doxycycline). In certain embodiments, the inducing agent is a reverse tetracycline-controlled transactivator (rtTA) (e.g., M2-rtTA, rtTA3 or rtTA4). In certain embodiments, the rtTA is rtTA3 comprising an amino acid sequence that is at least 70% (e.g., at least 75%, 80%, 85%, 90%, 95%, 98%, 99%, or 100%) identical to SEQ ID NO: 20. In certain embodiments, rtTA3 is encoded by a nucleic acid sequence that is at least 70% (e.g., at least 75%, 80%, 85%, 90%, 95%, 98%, 99%, or 100%) identical to SEQ ID NO: 19. In certain embodiments, the rtTA is rtTA4 and comprises an amino acid sequence that is at least 70% (e.g., at least 75%, 80%, 85%, 90%, 95%, 98%, 99%, or 100%) identical to SEQ ID NO: 27. In certain embodiments, rtTA4 is encoded by a nucleic acid sequence that is at least 70% (e.g., at least 75%, 80%, 85%, 90%, 95%, 98%, 99%, or 100%) identical to SEQ ID NO: 26.

In certain embodiments, the inducing agent is capable of inducing expression of expression of the first nucleic acid (e.g., OCT4), second nucleic acid (e.g., SOX2), third nucleic (e.g., KLF4), or any combination thereof from the inducible promoter in the absence of a tetracycline (e.g., doxycycline). In certain embodiments, the inducing agent is tetracycline-controlled transactivator ((TA).

In certain embodiments, the nucleic acid molecule of the present disclosure comprises a constitutive promoter, for example, one or more of CP1, CMV, EF1 alpha, SV40, PGK1, Ubc, human beta actin, CAG, Ac5, polyhedrin, TEF1, GDS, CaM3 5S, Ubi, Hl, and/or U6 promoter. The constitutive promoter may be operably linked to nucleic acid sequences encoding OCT4, KLF4, SOX2, an inducing agent, or a combination thereof. In some embodiments, the nucleic acid molecule comprises one constitutive promoter. In some embodiments, the nucleic acid molecule comprises more than one constitutive promoter.

In certain embodiments, the nucleic acid molecule of the present disclosure comprises an SV40-derived terminator sequence. In certain embodiments, the SV40-derived sequence is at least 70% (e.g., at least 75%, 80%, 85%, 90%, 95%, 98%, 99%, or 100%) identical to SEQ ID NO: 12 or 30.

In certain embodiments, the nucleic acid molecule of the present disclosure comprises a separator sequence, which may be useful in producing two separate amino acid sequences from one transcript. The separator sequence may encode a self-cleaving peptide (e.g., 2A peptide, including a 2A peptide sequence that is at least 70% (e.g., at least 75%, 80%, 85%, 90%, 95%, 98%, 99%, or 100%) identical to SEQ ID NO: 8 or 10). In certain embodiments, the separator sequence is an Internal Ribosome Entry Site (IRES).

### Viral Delivery Vehicles

A virus may be used as a delivery vehicle. In some embodiments, the expression vector is a viral expression vector. In some embodiments, the expression vector is a viral expression vector, wherein the viral vector is a lentivirus, a retrovirus, an adenovirus, alphavirus, vaccinia virus, or an adeno-associated virus (AAV) vector. The virus may include a protein that binds to a target such as a liver cell or protein of a liver cell.

In certain embodiments, a nucleic acid molecule herein includes a viral vector (e.g., a lentiviral, a retroviral, or an adeno-associated virus (AAV) vector). An AAV vector of the present disclosure generally comprises inverted terminal repeats (ITRs) flanking a transgene of interest (e.g., a nucleic acid sequence encoding OCT4, SOX2, KLF4, an inducing agent, or a combination thereof). In some embodiments, the distance between two inverted terminal repeats is less than 5.0 kilobases (kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb).

In another aspect, the present disclosure provides recombinant viruses. The recombinant viruses can include one or more lentivirus, adenovirus, retrovirus, herpes virus, alphavirus, vaccinia virus or adeno-associated virus (AAV) comprising any of the expression vectors described herein. In certain embodiments, the recombinant virus encodes a transcription factor selected from OCT4; KLF4; SOX2; and any combination thereof. In certain embodiments, the recombinant virus encodes two or more transcription factors selected from the group consisting of OCT4, KLF4, and SOX2. In certain embodiments, the recombinant virus encodes OCT4 and SOX2, OCT4 and KLF4, or SOX2 and KLF4. In certain embodiments, the recombinant virus encodes OCT4, KLF4, and SOX2. In certain embodiments, the recombinant virus encodes four or more transcription factors, for example OCT4, SOX2, KLF4, and another transcription factor.

In some embodiments, an AAV vector comprising a nucleic acid molecule encoding OCT4, SOX2, and KLF4 is an AAV2-TRE-OSK vector and the AAV vector comprising a nucleic acid molecule encoding rtTA is an AAV2-CMV-rtTA3. The AAV composition may include an AAV2-TRE-OSK vector and an AAV2-CMV-rtTA3 vector. The compositions or methods herein may include the AAV2-TRE-OSK vector and the AAV2-CMV-rtTA3 vector in the same or in separate compositions.

In some embodiments, an AAV vector comprising a nucleic acid molecule encoding OCT4, SOX2, and KLF4 is an AAV2-TRE-OSK vector and the AAV vector comprising a nucleic acid molecule encoding rtTA is an AAV2-CMV-rtTA4. The AAV composition may include an AAV2-TRE-OSK vector and an AAV2-CMV-rtTA4 vector. The compositions or methods herein may include the AAV2-TRE-OSK vector and the AAV2-CMV-rtTA4 vector in the same composition or in separate compositions.

### Methods of Preparing AAV-OSK Vectors

Provided herein are methods for recombinant preparation of an AAV. In some embodiments, the method comprises introducing one or more vectors as contemplated herein into a cell under conditions whereby the AAV is produced. The cell may include a population of cells. The population of cells may include any suitable cells as understood in the art, including for example HEK293 cells, HEK293T cells, COS cells, CHO cells, BHK cells, HeLa cells, and the like. The one or more vectors may be introduced into the cell using one or more suitable techniques including for example transfection, transduction, and/or infection. Exemplary methods for recombinant preparation of the AAV include transient transfection (e.g., with one or more transfer plasmids containing a first, and a second, and optionally a third vector as described herein), viral infection (e.g., with one or more recombinant helper viruses, such as a adenovirus, poxvirus (such as vaccinia virus), herpes virus (including HSV, cytomegalovirus, or baculovirus, containing a first, and a second, and optionally a third vector as described herein)), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more AAV capsid proteins as described herein, and with an AAV genome as described herein being delivered in the form of a plasmid or a recombinant helper virus). The first vector may include one or more nucleic acid sequences expressing OCT4, SOX2, and/or KLF4 encoded by one or more of SEQ ID NOs: 13, 14, 15, and 35. The second vector may include one or more nucleic acid sequences expressing transactivator 3, for example SEQ ID NO: 21 or 36. SEQ ID NO: 21 may include pAAV2-CMV-rtTA3 (VP16) and be an example of a vector sequence encoding rtTA. Alternatively, the second vector may include one or more nucleic acid sequences expressing transactivator 4, for example SEQ ID NO: 28 or 37. SEQ ID NO: 28 may include pAAV2-CMV-rtTA4 and be an example of a vector sequence encoding rtTA.

Further provided herein are methods for generating an AAV comprising modifying a cell to express one or more plasmids. The one or more plasmids may include one or more AAV2 Rep-Cap plasmids, one or more helper plasmids, and one or more transfer plasmids. The one or more transfer plasmids may include a first transfer plasmid including one or more nucleic acids encoding one or more of: OCT4, SOX2, and KLF4; a second transfer plasmid including one or more nucleic acids encoding transactivator 3; or a transfer plasmid including one or more nucleic acids encoding one or more of: OCT4, SOX2, KLF4, and transactivator 3. The one or more transfer plasmids may also include a first transfer plasmid including one or more nucleic acids encoding one or more of: OCT4, SOX2, and KLF4; a second transfer plasmid including one or more nucleic acids encoding transactivator 4; or a transfer plasmid including one or more nucleic acids encoding one or more of: OCT4, SOX2, KLF4, and transactivator 4.

### Nanoparticles

In some embodiments, the composition comprises nanoparticles. In some embodiments, the composition comprises a polymer or polymers. The nanoparticles may include one or more polynucleotides herein. The nanoparticles or a polymer may include polyethyleneimine (PEI) particles. The nanoparticles may include lipids.

The nanoparticles may include lipid nanoparticles (LNPs). In some embodiments, the composition comprises an LNP. In some embodiments, the composition comprises LNP. In some embodiments, the LNPs comprise a cationic lipid, an ionizable lipid, a helper lipid, a polyethylene glycol (PEG) conjugated (PEGylated) lipid, a cholesterol-based lipid, or a combination thereof. The nanoparticle or LNP may include lipids. Some examples may include an ionizable lipid (e.g., an amino lipid, (ii) a sterol or other structural lipid, (iii) a non-cationic helper lipid or phospholipid, (iv) a payload comprising the one or more nucleic acids, or (v) a PEG-lipid, or a combination thereof. Some examples of nanoparticles, lipids, lipid nanoparticles, and methods of making lipid nanoparticles may be included in any of the following, which disclosure is hereby incorporated by reference: WO2016118697A1, WO2021026358A1, WO2021226597A2, and WO2022067446.

LNPs may be used to for passive targeting to hepatocytes. In some embodiments, LNPs interact with serum proteins and acquire a protein-coated interface (e.g. protein corona) upon their intravenous injection. In some embodiments, apolipoprotein E (ApoE) is adsorbed on LNPs. ApoE may be an endogenous ligand for the LDLR family and heparan sulfate proteoglycans (HSPGs). An ApoE-coated LNP may be taken up by ApoE-LDLR-mediated endogenous pathway in hepatocytes.

Some embodiments of a nanoparticle or LNP include a cationic lipid. Some such examples may include N-[1-(2,3-dioleyloxy) propyl]-N,N,N-trimethylammonium chloride (DOTMA), N-[1-(2,3-dioleoyloxy)propyl]-N,N,N-trimethylammonium chloride (DOTAP), 1,2-dioleoyl-sn-glycero-3-ethylphosphocholine (DOEPC), 1,2-dilauroyl-sn-glycero-3-ethylphosphocholine (DLEPC), 1,2-dimyristoyl-sn-glycero-3-ethylphosphocholine (DMEPC), 1,2-dimyristoleoyl-sn-glycero-3-ethylphosphocholine (14:1), NI-[2-((IS)-1-[(3-aminopropyl)amino]-4-[di(3-amino-propyl)arrdno]butylcarboxarrddo)emyl]-3,4-di [oleyloxy]-benzamide (MVL5), Dioctadecylamido-glycylspermine (DOGS), 3b N—(N\hat{ }N′-dimethylaminoethyl)carbamoyl]cholesterol (DC-Choi), Dioctadecyldimethylammonium Bromide (DDAB), SAINT-2, Ai-methyl-4-(dioleyl)methylpyridinium, 1,2-dimyristyloxypropyl-3-dimethylhydroxyethylammonium bromide (DMRIE), 1,2-dioleoyl-3-dimethyl-hydroxyethyl ammonium bromide (DORIE), 1,2-dioleoyloxypropyl-3-dimethylhydroxyethyl ammonium chloride (DORI), Di-alkylated Amino Acid (DILA2), C18:1-norArg-C16, Dioleyldimethylammonium chloride (DODAC), 1-palmitoyl-2-oleoyl-OT-glycero-3-emylphosphocholine (POEPC), 1,2-dimyristoleoyl-OT-glycero-3-ethylphosphocholine (MOEPC), (R)-5-(dimethylamino) pentane-1,2-diyl dioleate hydrochloride (DODAPen-Cl), (R)-5-guanidinopentane-1,2-diyl dioleate hydrochloride (DOPen-G), (R)-Ai,Ai,Ai-trimethyl-4,5-bis(oleoyloxy) pentan-1-aminium chloride (DOTAPen). A combination of cationic lipids may be included.

Some embodiments of a nanoparticle or LNP include an ionizable lipid. Some such embodiments may include a racemic mixture of the amino lipid, e.g., a mixture comprising a (R)-enantiomer and an(S)-enantiomer of an amino lipid. Some embodiments include an ionizable lipid having a chiral center. Some embodiments include an ionizable lipid comprising more than one branched alkyl chains. Some embodiments include a cyclic-substituted amino lipid, a carbocyclic-substituted amino lipid, a cycloalkenyl-substituted amino lipid, an amino lipid having a chiral center, an amino lipid comprising more than one branched alkyl chains, a cyclic-substituted amino lipid, a carbocyclic-substituted amino lipid, a cycloalkenyl-substituted amino lipid, a cyclobutenyl-substituted amino lipid, a cyclobutene-1,2-dione-substituted amino lipid, or a squaramide-substituted amino lipid, e.g., an amino lipid comprising a squaramide group. An ionizable lipid may include dioctadecyldimethylammonium bromide (DDAB), 1,2-dilinoleyloxy-3-dimethylaminopropane (DLinDMA), 2,2-dilinoleyl-4-(2dimethylaminoethyl)-[1,3]-dioxolane (DLin-KC2-DMA), heptatriaconta-6,9,28,31-tetraen-19-yl 4-(dimethylamino) butanoate (DLin-MC3-DMA), 1,2-Dioleoyloxy-3-dimethylaminopropane (DODAP), 1,2-Dioleyloxy-3-dimethylaminopropane (DODMA), or morpholinocholesterol (Mo-CHOL). An ionizable lipid may include cholesteryl hemisuccinate (CHEMS), phosphatidylserine, or palmitoylhomoserine,-tocopherol hemisuccinate. A combination of ionizable lipids may be included.

Some embodiments of a nanoparticle or LNP include a non-cationic helper lipid or phospholipid comprising a compound. Some such examples 1,2-distearoyl-OT-glycero-3-phosphocholine (DSPC), 1,2-dipalmitoyl-OT-glycero-3-phosphocholine (DPPC), 1,2-dimyristoyl-OT-glycero-3-phosphocholine (DMPC), 1,2-dioleoyl-sn-glycero-3-phosphoethanolamine (DOPE), 1,2-dilauroyl-sn-glycero-3-phosphoethanolamine (DLPE), 1,2-dimyristoyl-sn-glycero-3-phosphoethanolamine (DMPE), or 1,2-diphytanoyl-sn-glycero-3-phosphoethanolamine (DPHyPE). A helper lipid may include DSPC, DPPC, DMPC, DMPE, or DOPC. A helper lipid may include 1,2-dioleoyl-sn-glycero-sn-3-phosphatidylcholine (DOPC) or 1,2-dioleoyl-sn-glycero-3-phosphatidyl-ethanolamine (DOPE). A helper lipid may include DOPC. A helper lipid may include DOPE. A combination of non-cationic or helper lipids may be included.

Some embodiments of a nanoparticle or LNP include a PEGylated or PEG-modified lipid. Some such examples may include a PEGylated phosphatidic acid, a PEG-modified ceramide, a PEG-modified dialkylamine, a PEG-modified diacylglycerol, or a PEG-modified dialkylglycerol. Some examples of PEGylated lipids may include PEG-c-DOMG, PEG-DMG, PEG-DLPE, PEG-DMPE, PEG-DPPC, or PEG-DSPE lipid. The PEGylated lipid may include a PEGylated 1,2-dimyristoyl-rac-glycero-3-methoxy (DMG). The PEGylated lipid may include 1,2-dimyristoyl-rac-glycero-3-methoxypolyethylene glycol (PEG-DMG, also referred to as DMG-PEG). In some embodiments, the PEGylated lipid includes 1,2-dimyristoyl-rac-glycero-3-methoxypolyethylene glycol-2000 (DMG-PEG-2000 or DMG-PEG-2K). Some examples of a PEGylated lipid may include N-(Carbonyl-methoxypolyethyleneglycol 2000)-1,2-dimyristoyl-5,ra-glycero-3-phosphoethanolamine (DMPE-PEG 2,000), N-(Carbonyl-methoxypolyethyleneglycol 2000)-1,2-distearoyl-s,re-glycero-3-phosphoethanolamine (DSPE-PEG 2,000) polyethylene gycol-dimyristolglycerol (PEG-DMG), polyethylene glycol-distearoyl glycerol (PEG-DSG), or N-octanoyl-sphingosine-1-{succinyl [methoxy (polyethylene glycol)2000]} (C8 PEG2000 ceramide). A combination of PEGylated lipids may be included.

Some embodiments of a nanoparticle or LNP include cholesterol. Some embodiments of a nanoparticle or LNP include a cholesterol derivative. Some embodiments of a nanoparticle or LNP include a substituted cholesterol derivative.

In some embodiments, LNPs are actively targeted to a liver or a liver cell. Some embodiments of an LNP include a targeting moiety. A targeting moiety may increase delivery to a liver cell such as a hepatocyte or hepatic stellate cell (HSC). An example of a targeting moiety may include a N-acetylgalactosamine (GalNAc) moiety. The GalNAc or another moiety may target hepatocytes, e.g. via binding to a liver cell protein such as an asialoglycoprotein receptor. An example of a targeting moiety may include an antibody, antibody fragment, or scFv. The antibody, antibody fragment, or scFv may target a liver protein such as a hepatocyte protein (e.g. an asialoglycoprotein receptor) or a HSC surface receptor. A targeting moiety may include a targeting ligand. An example of a targeting moiety may include a retinoid (e.g. BMS-986263). The targeting moiety may be conjugated to an aspect of the LNP. A targeting moiety may be conjugated to a lipid. A targeting moiety may be conjugated to a nucleic acid (e.g. to one or more nucleic acids encoding partial reprogramming factors). Some embodiments include a retinoid conjugated LNP. The LNP may include a lipid formulation that targets liver cells or a liver of a subject. In some embodiments, ApoE or an ApoE analog is used as a targeting moiety. Activated HSC may include any of various cell surface receptors, such as retinol binding protein (RBP) receptor, integrin αvβ3, M6P/IGF II receptor, PDGF receptor-β, hyaluronic acid (HA) receptor CD44 and CXCR4, any of which may be targeted by a targeting moiety.

In some embodiments, a lipid nanoparticle is less than about 200 nm in size. For example, a lipid nanoparticle may be from about 30 nm to about 150 nm in size. In certain variations, the size of the lipid nanoparticle (e.g., between about 30 nm and about 150 nm) may help facilitate delivery to a liver.

### Methods of Treatment

Some embodiments relate to a method of treating a subject having a liver disease. Such methods may include administering to the subject an effective amount of a pharmaceutical composition comprising one or more polynucleotides encoding OCT4, SOX2 and KLF4 (together, OSK). In some embodiments, the one or more polynucleotides do not encode MYC proto-oncogene (c-Myc). Some embodiments include a method of treating a subject having a liver disease, comprising: administering to the subject an effective amount of a pharmaceutical composition comprising one or more polynucleotides encoding OCT4, SOX2 and KLF4, and not encoding c-Myc.

Some embodiments relate to treating a subject having a liver disease, reversing epigenetic change in a subject having a liver disease, or reverting a liver disease to an earlier stage of disease in a subject. Some embodiments relate to treating a subject in need of treatment for MASLD or MASH, reversing epigenetic change in a subject in need of treatment for MASLD or MASH, or reverting MASLD or MASH to an earlier stage of disease in a subject in need thereof. Treating may include reversing epigenetic change. Reversing epigenetic change in a liver or liver disease may include reverting a liver or liver disease to an earlier stage of disease. Reversing epigenetic change may include reverting a marker of epigenetic change associated with the liver disease to a state associated with a healthy liver or to a state associated with a stage of the disease that is earlier than a stage at which the pharmaceutical composition is administered. Reversing epigenetic change may include reverting a marker of epigenetic change associated with MASLD or MASH to a state associated with a healthy liver or to a state associated with a stage of MASLD or MASH that is earlier than a MASLD or MASH stage at which the pharmaceutical composition is administered. The marker of epigenetic change may include DNA methylation or another marker of epigenetic change.

Hepatocytes or hepatic stellate cells (HSCs) may be useful targets for the treatment of liver diseases such as MASLD or MASH. In some embodiments, OSK alleviates a liver disease (e.g. MASLD or MASH) via rejuvenation of hepatocytes. In some embodiments, OSK alleviates a liver disease (e.g. MASLD or MASH) by reducing fibrogenesis of HSCs.

The treatment may include rejuvenating cells. For example, some embodiments relate to a method of rejuvenating cells of a subject having a liver disease. The rejuvenated cells may be cells of a subject administered a pharmaceutical composition herein. The rejuvenation may include a reversal of epigenetic change. For example, some embodiments relate to a method of reversing epigenetic change in a subject with a liver disease. Some embodiments of any such method include administering to the subject an effective amount of a composition comprising one or more polynucleotides encoding OCT4, SOX2 and KLF4.

Provided herein, in some embodiments, are methods or compositions that reverse or alleviate an epigenetic change, or revert a disease to an earlier state. In some embodiments, alleviation of an epigenetic change is partial. In some embodiments, alleviation of an epigenetic change is complete. Some methods or compositions herein may revert a liver disease to an earlier stage of disease. The epigenetic change may be in diseased tissue (e.g. diseased liver tissue) relative to non-diseased tissue (e.g. non-diseased liver tissue), or relative to a baseline tissue (e.g. liver tissue before the epigenetic change) prior to disease development. The epigenetic change may be in diseased cell (e.g. diseased liver cell) relative to non-diseased cell (e.g. non-diseased liver cell), or relative to a baseline cell (e.g. liver cell) prior to disease development. The epigenetic change may include a marker of cell aging. The epigenetic change may include DNA methylation. The DNA methylation may include methylation at a CpG site such as a site identified in Johnson N D, et al. Differential DNA methylation and changing cell-type proportions as fibrotic stage progresses in NAFLD. Clin Epigenetics. 2021; 13 (1): 152; or Bi H, et al. Whole-genome DNA methylation and gene expression profiling in the livers of mice with nonalcoholic steatohepatitis. Life Sci. 2023:329:121951, which are incorporated herein by reference in their entirety. Some examples of epigenetic changes that may occur, and may be reversed or alleviated are included in Intl. Publ. No. WO2020/069373, incorporated by reference herein in its entirety. Some embodiments include reversing or alleviating such change in a liver cell or liver tissue of a subject that has a liver disease.

Reversing an epigenetic change may include reversing an epigenetic change in diseased cell or tissue (e.g. diseased liver cell or tissue). The reversal may be relative to a baseline (e.g. before treatment or administration with a composition herein). The reversal may include reverting a diseased cell or tissue back to a pre-diseased state. For example, reversing an epigenetic change may include reverting a marker of cell aging or a DNA methylation status to a pre-disease or youthful state. The epigenetic change may be reversed in diseased tissue (e.g. diseased liver tissue). The epigenetic change may be reversed in diseased cell (e.g. diseased liver cell).

Provided herein, in some embodiments, are methods for treating a subject in need of treatment for metabolic dysfunction-associated steatotic liver disease (MASLD) or metabolic dysfunction-associated steatohepatitis (MASH), the method comprising administering to the subject a pharmaceutically effective amount of a pharmaceutical composition comprising an expression vector encoding three transcription factors, wherein the three transcription factors consist of OCT4, SOX2, and KLF4.

Provided herein are methods for treating a liver disease in a subject in need thereof wherein the method includes administering to the subject one or more agents for upregulating OCT4, SOX2, KLF4, and/or one or more combinations thereof to the subject. In some embodiments, the one or more agents do not upregulate c-Myc. The one or more agents for upregulating OCT4, SOX2, and KLF4 (OSK) may include one or more means for inducing expression of OSK, including DNA, RNA, small molecules, and the like. In some embodiments, the methods include administering to the subject one or more nucleic acid molecules as contemplated herein. In some embodiments the one or more nucleic acid molecules include a nucleic acid molecule system having at least two nucleic acid molecules.

In some embodiments, OSK therapy can cause or contribute to a decrease in ALT in plasma in a subject. In some embodiments, OSK therapy can cause or contribute to a decrease in AST in plasma in a subject. In some embodiments, OSK therapy can cause or contribute to a decrease in total bile acids in plasma in a subject. In some embodiments, OSK therapy can cause or contribute to a decrease in total cholesterol in plasma in a subject. In some embodiments, OSK therapy can cause or contribute to an improvement of at least 1 point in NAFLD score in a subject. In some embodiments, OSK therapy reduced the percentage of hepatocytes with lipid droplets in a subject. In some embodiments, OSK therapy can cause or contribute to a decrease in liver weight in a subject. In some embodiments, OSK therapy can cause or contribute to an improvement of at least 1 point in steatosis score in a subject. In some embodiments, OSK therapy does not cause a decrease in body weight of a subject.

### Inducing Partial Reprogramming Factor Expression

Some embodiments include inducing expression of reprogramming factors. Some embodiments include inducing expression of partial reprogramming factors. The partial reprogramming factors may include OCT4, SOX2, and KLF4. The induction may be in a subject. The induction may include expressing or increasing expression of one or more polynucleotides encoding partial reprogramming factors. The induction may increase in a reprogramming factor measurement, relative to a baseline reprogramming factor measurement. Some embodiments include administering to the subject an inducing agent to induce expression of OCT4, SOX2, and KLF4 in a subject.

In some embodiments of the methods for treating a liver disease in a subject, the agent for upregulating OSK expression includes at least one nucleic acid molecule encoding OSK as described herein above. The nucleic acid molecule encoding OSK does not comprise a nucleic acid sequence encoding c-myc. The nucleic acid molecule encoding OSK may be an adeno-associated viral (AAV) vector. According to some embodiments, the methods further comprise administering to the subject a nucleic acid molecule encoding a reverse tetracycline-controlled transactivator (rtTA). The nucleic acid molecule encoding a reverse tetracycline-controlled transactivator (rtTA) may be an AAV vector. In some embodiments, the AAV vector comprising a nucleic acid molecule encoding reverse tetracycline-controlled transactivator (rtTA) is not the same AAV vector as the AAV vector comprising a nucleic acid molecule encoding OCT4, SOX2, and KLF4.

The nucleic acid molecule encoding OCT4, SOX2, and KLF4 is operably linked to an inducible promoter. In some embodiments, the inducible promoter is induced by a tetracycline class antibiotic. Tetracycline class antibiotics are known in the art and include, for example, tetracycline, chlortetracycline, oxytetracycline, demeclocyline, lymecycline, meclocycline, methacycline, minocycline, rolitetracycline, doxycycline, tigecycline, eravacycline, sarecycline, and omadacycline. Doxycycline is an exemplary tetracycline class antibiotic. In some embodiments, the inducible promoter is a tetracycline class antibiotic response element (TRE) including for example a TRE2 promoter.

The reverse tetracycline-controlled transactivator (rtTA) may be rtTA3, rtTA4, or combinations thereof.

In some embodiments, the nucleic acid molecule encoding rtTA is operably linked to a constitutive promotor including one or more of CP1, CMV, EF1 alpha, SV40, PGK1, Ubc, human beta actin, CAG, Ac5, polyhedrin, TEF1, GDS, CaM3 5S, Ubi, Hl, and/or U6 promoter. In some embodiments, the nucleic acid molecule encoding rtTA is operably linked a CMV promoter.

In some embodiments, the AAV vector is serotype-2 (AAV2). In some embodiments, the AAV vector is a hybrid vector comprising capsid proteins from one or more serotypes including AAV1, AAV2, AAV5, AAV6, AAV7, AAV8 and AAV9 (e.g., AAV2/2, AAV2/6. AAV2/1, AAV2/5, AAV2/7, AAV2/8 and AAV2/9). In some embodiments, the AAV is an AAV6 serotype AAV. In some embodiments, AAV6 vectors are useful in delivering a nucleic acid payload to cells of the liver, such as liver stellate cells. In some embodiments, the AAV is an AAV8 serotype AAV. In some embodiments, AAV8 vectors are useful in delivering a nucleic acid payload to cells of the liver, such as hepatocytes.

In some embodiments, the AAV vector comprising a nucleic acid molecule encoding OCT4, SOX2, and KLF4 comprises a self-cleaving peptide, for example a 2A peptide.

In some embodiments, the AAV vector comprising a nucleic acid molecule encoding OCT4, SOX2, and KLF4 comprises inverted terminal repeats (ITRs) flanking the first nucleic acid. In some embodiments, the AAV vector comprising a nucleic acid molecule encoding OCT4, SOX2, and KLF4 comprises inverted terminal repeats (ITRs) flanking the second nucleic acid. In some embodiments, the AAV vector comprising a nucleic acid molecule encoding OCT4, SOX2, and KLF4 comprises inverted terminal repeats (ITRs) flanking the third nucleic acid. In some embodiments, the AAV vector comprising a nucleic acid molecule encoding OCT4, SOX2, and KLF4 comprises inverted terminal repeats (ITRs) flanking one or more combinations of the first nucleic acid, the second nucleic acid, and/or the third nucleic acid. In some embodiments, the distance between two inverted terminal repeats (ITRs) is less than 5.0 kilobases (kb) (e.g., less than 4.9 kb, less than 4.8 kb, less than 4.7 kb, less than 4.6 kb, less than 4.5 kb, less than 4.4 kb, less than 4.3 kb, less than 4.2 kb, less than 4.1 kb, less than 4 kb, less than 3.5 kb, less than 3 kb, less than 2.5 kb, less than 2 kb, less than 1.5 kb, less than 1 kb, or less than 0.5 kb). In some embodiments, the distance between two ITRs is 4.7 kb or less.

The method can further include administering an inducing agent to the subject. The inducing agent can include for example a tetracycline-controlled transactivator (tTA). In certain aspects, the inducing agent is capable of inducing expression of expression of the first nucleic acid (e.g., OCT4), the second nucleic acid (e.g., SOX2), the third nucleic (e.g., KLF4), or any combination thereof from the inducible promoter in the absence of a tetracycline (e.g., doxycycline).

In some embodiments, the AAV-OSK vector comprising a nucleic acid molecule encoding OCT4, SOX2, and KLF4 comprises nucleic acid elements in a specific order. For example, the AAV vector comprising a nucleic acid molecule encoding OCT4, SOX2, and KLF4 can include elements in the following order: a) a first inverted terminal repeat sequence (ITR) sequence; b) a TRE2 promoter sequence; c) an OCT4 sequence: d) a P2A cleavage sequence; e) a SOX2 sequence: f) a T2A cleavage sequence; g) a KLF4 sequence; h) an SV-40-derived terminator sequence; and i) a second inverted terminal repeat (ITR) sequence, as described, for example in Intl. Application No. PCT/US2019/053545, published as Intl. Publ. No. WO2020/069373 titled CELLULAR REPROGRAMMING TO REVERSE AGING AND PROMOTE ORGAN AND TISSUE REGENERATION, incorporated by reference herein in its entirety.

In certain embodiments, the encoded OCT4 comprises an amino acid sequence at least 70% (e.g., at least 75%, 80%, 85%, 90%, 95%, 98%, 99%, or 100%) identical to SEQ ID NO: 2. In certain embodiments, the nucleic acid sequence encoding OCT4 is at least 70% (e.g., at least 75%, 80%, 85%, 90%, 95%, 98%, 99%, or 100%) identical to SEQ ID NO: 1. In certain embodiments, the encoded SOX2 comprises an amino acid sequence at least 70% (e.g., at least 75%, 80%, 85%, 90%, 95%, 98%, 99%, or 100%) identical to SEQ ID NO: 4. In certain embodiments, the nucleic acid sequence encoding SOX2 is at least 70% (e.g., at least 75%, 80%, 85%, 90%, 95%, 98%, 99%, or 100%) identical to SEQ ID NO: 3. In certain embodiments, the encoded KLF4 comprises an amino acid sequence at least 70% (e.g., at least 75%, 80%, 85%, 90%, 95%, 98%, 99%, or 100%) identical to SEQ ID NO: 6. In certain embodiments, the nucleic acid sequence encoding KLF4 is at least 70% identical (e.g., at least 75%, 80%, 85%, 90%, 95%, 98%, 99%, or 100%) to SEQ ID NO: 5. In some embodiments, the nucleic acid sequence encoding OCT4 is SEQ ID NO: 1, the nucleic acid sequence encoding SOX2 is SEQ ID NO: 3, and the nucleic acid sequence encoding KLF4 is SEQ ID NO: 5.

In some embodiments, the P2A sequence encodes for a polypeptide with the sequence ATNFSLLKQAGDVEENPGP (SEQ ID NO: 9). In some embodiments, the P2A sequence is GCCACGAACTTCTCTCTGTTAAAGCAAGCAGGAGATGTTGAAGAAAACCCCGGGCC T (SEQ ID NO: 8).

In some embodiments, the T2A sequence encodes a polypeptide of SEQ ID NO: 11. In some embodiments, the T2A sequence is GAGGGCAGGGGAAGTCTTCTAACATGCGGGGACGTGGAGGAAAATCCCGGCCCA (SEQ ID NO: 10).

In some embodiments, the TRE2 promoter sequence is SEQ ID NO: 7. In some embodiments, the TRE2 promoter sequence comprises at least one minimal CMV promoter sequence. In some embodiments, the at least one minimal SV40 promoter sequence includes SEQ ID NO: 12.

In some embodiments, the SV-40-derived terminator sequence includes SEQ ID NO: 12.

In some embodiments, the ITR sequence is SEQ ID NO: 16.

In some embodiments, the nucleic acid molecule encoding OCT4, SOX2, and KLF4 comprises SEQ ID NO: 13 or 14. In some embodiments, the nucleic acid molecule encoding OCT4, SOX2, and KLF4 comprises SEQ ID NO: 15.

The AAV vector comprising a nucleic acid molecule encoding OCT4, SOX2, and KLF4 and the AAV vector comprising a nucleic acid molecule encoding rtTA are administered sequentially or simultaneously.

### Liver Disease and Subjects

Disclosed herein, in some embodiments, are liver diseases. Examples of liver diseases may include metabolic dysfunction-associated steatotic liver disease (MASLD) or metabolic dysfunction-associated steatohepatitis (MASH). In some embodiments, the liver disease is chronic. In some embodiments, the liver disease comprises liver steatosis. In some embodiments, the liver disease comprises MASLD or MASH. In some embodiments, the liver disease comprises liver fibrosis. In some embodiments, the liver disease comprises cirrhosis.

In some embodiments, the subject has the liver disease. In some embodiments, the subject is suspected of having the liver disease. In some embodiments, the subject has or is suspected of having MASLD or MASH. Some embodiments include identifying the subject as having the liver disease before administering a pharmaceutical composition to the subject. Some embodiments include identifying the subject as suspected of having the liver disease before administering a pharmaceutical composition to the subject.

In some embodiments, such as before treatment, a subject has a symptom of a liver disease. For example, the subject may have liver steatosis, fibrosis, or inflammation. The inflammation may include lobular inflammation or ballooning. The symptom may be assessed on a numerical scale. For example, the fibrosis may be determined to be on a scale of 0 to 4 (e.g. 0, 1, 2, 3, or 4), where 0 indicates no fibrosis, and 4 is indicative of severe fibrosis. A DNA methylation determination may be used to assess liver fibrosis.

In some embodiments, the subject is a vertebrate. In some embodiments, the subject is an animal. In some embodiments, the subject is a mammal. In some embodiments, the subject is a primate. In some embodiments, the subject is a human. In some embodiments, the subject is male. In some embodiments, the subject is female.

### Administration

A composition (e.g. nucleic acid molecules disclosed herein) can be administered to a subject by any appropriate route including, without limitation, intravenous, intraperitoneal, subcutaneous, intramuscular, intranasal, topical, or intradermal routes. In certain embodiments, the composition is formulated for administration via intravenous injection or subcutaneous injection. In some embodiments, the dual vector system including an AAV vector comprising a nucleic acid molecule encoding OCT4, SOX2, and KLF4 and an AAV vector comprising a nucleic acid molecule encoding rtTA is administered intravenously or by injection. In some embodiments, the administration is intravenous. In some embodiments, the administration comprises an injection.

In some embodiments, one or more polynucleotides (e.g. a nucleic acid molecule) encoding OCT4, SOX2, and KLF4 and a nucleic acid molecule encoding rtTA (e.g., rtTA3, rtTA4, etc.) are administered at a ratio (nucleic acid molecule:nucleic acid molecule) of about 1:20, 1:19, 1:18, 1:17, 1:16, 1:15, 1:14, 1:13, 1:12, 1:11, 1:10; 1:9; 1:8; 1:7; 1:6; 1:5; 1:4; 1:3; 1:2; 1:1; 1:0.5; 2:1; 3:1; 4:1; 5:1; 6:1, 7:1; 8:1; 9:1; 10:1, 11:1, 12:1, 13:1, 14:1, 15:1, 16:1, 17:1, 18:1, 19:1, or 20:1. In some embodiments, the nucleic acid molecule encoding OCT4, SOX2, and KLF4 and the nucleic acid molecule encoding rtTA (e.g., rtTA3, rtTA4, etc.) are administered at a ratio of about 1:2; 1:1.9; 1:1.8; 1:1.7; 1:1.6; 1:1.5; 1:1.4; 1:1.3; 1:1.2; 1:1.1; 1:1; 1:0.9; 1:0.8; 1:0.7; 1:0.6; 1:0.5; 1:0.4; 1:0.3; 1:0.2; 1:0.1; 0.1:1; 0.2:1; 0.3:1; 0.4:1; 0.5:1, 0.6:1; 0.7:1; 0.8:1; 0.9:1; 1:1; 1.1:1; 1.2:1; 1.3:1; 1.4:1; 1.5:1; 1.6:1; 1.7:1; 1.8:1; 1.9:1 or 2:1. In some embodiments, the nucleic acid molecule encoding OCT4, SOX2, and KLF4 and the nucleic acid molecule encoding rtTA are administered at an about 1:1 ratio.

In some embodiments, the AAV vector comprising a nucleic acid molecule encoding OCT4, SOX2, and KLF4 and the AAV vector comprising a nucleic acid molecule encoding rtTA (e.g., rtTA3, rtTA4, etc.) are administered at a ratio (vector genome:vector genome (vg:vg)) of about 1:20, 1:19, 1:18, 1:17, 1:16, 1:15, 1:14, 1:13, 1:12, 1:11, 1:10; 1:9; 1:8; 1:7; 1:6; 1:5; 1:4; 1:3; 1:2; 1:1; 1:0.5; 2:1; 3:1; 4:1; 5:1; 6:1, 7:1; 8:1; 9:1; 10:1, 11:1, 12:1, 13:1, 14:1, 15:1, 16:1, 17:1, 18:1, 19:1, or 20:1. In some embodiments, the AAV vector comprising a nucleic acid molecule encoding OCT4, SOX2, and KLF4 and the AAV vector comprising a nucleic acid molecule encoding rtTA are administered at an about 1:1 (vg:vg) ratio. In some embodiments, the AAV vector comprising a nucleic acid molecule encoding OCT4, SOX2, and KLF4 and the AAV vector comprising a nucleic acid molecule encoding rtTA (e.g., rtTA3, rtTA4, etc.) are administered at a ratio (vg/vg) of about 1:10; 1:9; 1:8; 1:7; 1:6; 1:5; 1:4; 1:3; 1:2; 1:1; 1:0.5; 2:1; 3:1; 4:1; 5:1; 6:1, 7:1; 8:1; 9:1; or 10:1. In some embodiments, the AAV vector comprising a nucleic acid molecule encoding OCT4, SOX2, and KLF4 and the AAV vector comprising a nucleic acid molecule encoding rtTA (e.g., rtTA3, rtTA4, etc.) are administered at a ratio (vg/vg) of about 1:2; 1:1.9; 1:1.8; 1:1.7; 1:1.6; 1:1.5; 1:1.4; 1:1.3; 1:1.2; 1:1.1; 1:1; 1:0.9; 1:0.8; 1:0.7; 1:0.6; 1:0.5; 1:0.4; 1:0.3; 1:0.2; 1:0.1; 0.1:1; 0.2:1; 0.3:1; 0.4:1; 0.5:1, 0.6:1; 0.7:1; 0.8:1; 0.9:1; 1:1; 1.1:1; 1.2:1; 1.3:1; 1.4:1; 1.5:1; 1.6:1; 1.7:1; 1.8:1; 1.9:1 or 2:1. In some embodiments, the AAV vector comprising a nucleic acid molecule encoding OCT4, SOX2, and KLF4 and the AAV vector comprising a nucleic acid molecule encoding rtTA are administered at an about 1:1 (vg/vg) ratio. According to some embodiments of the disclosed methods, the AAV vector comprising a nucleic acid molecule encoding OCT4, SOX2, and KLF4 comprises SEQ ID NO: 13, 14, or 35. According to some embodiments, the AAV vector comprising a nucleic acid molecule encoding rtTA comprises SEQ ID NO: 19, 26, 36, or 37. According to some embodiments of the disclosed methods, the AAV vector comprising a nucleic acid molecule encoding OCT4, SOX2, and KLF4 comprises SEQ ID NO: 13, 14, or 35 and the AAV vector comprising a nucleic acid molecule encoding rtTA comprises SEQ ID NO: 19, 26, 36, or 37. According to some embodiments of the disclosed methods, the AAV vector comprising a nucleic acid molecule encoding OCT4, SOX2, and KLF4 comprises SEQ ID NO: 35 and the AAV vector comprising a nucleic acid molecule encoding rtTA comprises SEQ ID NO: 36 or 37.

In some embodiments, a concentration of the AAV2-TRE-OSK vector to be administered in accordance with the disclosed methods includes an amount in the range of from about 1×1010 vg/mL to about 2×1013 vg/mL, for example, 1×1012 vg/mL to about 2×1012 vg/mL. For example, the effective amount of the AAV2-TRE-OSK vector can include from about 1.0×1012 vg/mL to about 1.1×1012 vg/mL, from about 1.1×1012 vg/mL to about 1.2×1012 vg/mL, from about 1.2×1012 vg/mL to about 1.3×1012 vg/mL, from about 1.3×1012 vg/mL to about 1.4×1012 vg/mL, from about 1.4×1012 vg/mL to about 1.5×1012 vg/mL, from about 1.5×1012 vg/mL to about 1.6×1012 vg/mL, from about 1.6×1012 vg/mL to about 1.7×1012 vg/mL, from about 1.7×1012 vg/mL to about 1.8×1012 vg/mL, from about 1.8×1012 vg/mL to about 1.9×1012 vg/mL, from about 1.9×1012 vg/mL to about 2.0×1012 vg/mL and any and all increments therebetween.

In some embodiments, a concentration of the AAV2-CMV-rtTA3 vector administered in accordance with the disclosed methods includes an amount in the range of from about 1×1010 vg/mL to about 2×1013 vg/mL, for example, about 1×1013 vg/mL to about 2×1013 vg/mL. For example, the effective amount of the AAV2-CMV-rtTA3 vector can include from about 1.0×1013 vg/mL to about 1.1×1013 vg/mL, from about 1.1×1013 vg/mL to about 1.2×1013 vg/mL, from about 1.2×1013 vg/mL to about 1.3×1013 vg/mL, from about 1.3×1013 vg/mL to about 1.4×1013 vg/mL, from about 1.4×1013 vg/mL to about 1.5×1013 vg/mL, from about 1.5×1013 vg/mL to about 1.6×1013 vg/mL, from about 1.6×1013 vg/mL to about 1.7×1013 vg/mL, from about 1.7×1013 vg/mL to about 1.8×1013 vg/mL, from about 1.8×1013 vg/mL to about 1.9×1013 vg/mL, from about 1.9×1013 vg/mL to about 2.0×1013 vg/mL and any and all increments therebetween.

In some embodiments, a concentration of the AAV2-CMV-rtTA4 vector administered in accordance with the disclosed methods includes an amount in the range of from about 1×1010 vg/mL to about 2×1013 vg/mL, for example, about 1×1013 vg/mL to about 2×1013 vg/mL. For example, the effective amount of the AAV2-CMV-rtTA4 vector can include from about 1.0×1013 vg/mL to about 1.1×1013 vg/mL, from about 1.1×1013 vg/mL to about 1.2×1013 vg/mL, from about 1.2×1013 vg/mL to about 1.3×1013 vg/mL, from about 1.3×1013 vg/mL to about 1.4×1013 vg/mL, from about 1.4×1013 vg/mL to about 1.5×1013 vg/mL, from about 1.5×1013 vg/mL to about 1.6×1013 vg/mL, from about 1.6×1013 vg/mL to about 1.7×1013 vg/mL, from about 1.7×1013 vg/mL to about 1.8×1013 vg/mL, from about 1.8×1013 vg/mL to about 1.9×1013 vg/mL, from about 1.9×1013 vg/mL to about 2.0×1013 vg/mL and any and all increments therebetween.

Embodiments of the methods further include administering to the subject an effective amount of an antibiotic. In some embodiments, the antibiotic includes tetracycline or doxycycline. In some embodiments, the antibiotic is administered at least one day prior to administering the AAV vector comprising a nucleic acid molecule encoding OCT4, SOX2, and KLF4 and the AAV vector comprising a nucleic acid molecule encoding rtTA.

The antibiotic may be administered when the AAV vector comprising a nucleic acid molecule encoding OCT4, SOX2, and KLF4 and the AAV vector comprising a nucleic acid molecule encoding rtTA are administered. The antibiotic may be administered at least one day following administration of the AAV composition comprising the AAV vector comprising a nucleic acid molecule encoding OCT4, SOX2, and KLF4 and the AAV vector comprising a nucleic acid molecule encoding rtTA. The antibiotic may be administered 2 days, 3 days, 4 days, 5 days, or more than 5 days following administration of the AAV composition comprising the AAV vector comprising a nucleic acid molecule encoding OCT4, SOX2, and KLF4 and the AAV vector comprising a nucleic acid molecule encoding rtTA.

The disclosed methods of treating a liver disease may include methods of administering to a subject in need thereof an effective amount of an AAV genome including one or more polynucleotide sequences expressing OCT4, SOX2, and KLF4 and an AAV genome comprising a nucleic acid sequence encoding transactivator 3 or transactivator 4.

### Combination Therapy

A composition for OSK therapy as disclosed herein (e.g. a composition including one or more nucleic acid molecules encoding OCT4, SOX2, and KLF4) can be administered to a subject in combination with one or more additional therapies, agents, or regimens. As used herein, the term “combination therapy” refers to administration to a subject of to two or more therapies, agents, or regimens such that the two or more therapies, agents, or regimens together treat, or contribute to treatment of, a disease, condition, or disorder of the subject (such as liver disease, e.g., MASH). In some embodiments, the two or more therapies, agents, or regimens can be administered simultaneously, sequentially, or in an overlapping manner. Those of skill in the art will appreciate that combination therapy includes but does not require that the two therapies, agents, or regimens are administered at the same time. Those of skill in the art will appreciate that combination therapy includes but does not require that the therapies, agents, or regimens are administered together in a single composition.

The present disclosure includes combination therapies comprising an OSK therapy and one or both of an obesity therapy and a liver disease therapy (e.g., a MASH therapy). In some embodiments, the present disclosure includes combination therapies including an OSK therapy and an obesity therapy. In some embodiments, the present disclosure includes combination therapies including an OSK therapy and a liver disease therapy (e.g., a MASH therapy). In some embodiments, the present disclosure includes combination therapies including an OSK therapy, an obesity therapy, and a liver disease therapy (e.g., a MASH therapy).

In various embodiments, a combination therapy of the present disclosure provides an additive effect for treatment of liver disease (e.g., MASH). In various embodiments, a combination therapy of the present disclosure provides a synergistic effect for treatment of liver disease (e.g., MASH). In various embodiments, a combination therapy of the present disclosure provides an adjunctive effect for treatment of liver disease (e.g., MASH). In various embodiments, an effect for treatment of liver disease, e.g., MASH can be measured by any of one or more metrics of disease provided herein or known in the art, including without limitation a single metric of disease provided herein, and can be measured in any representative human, animal, or in vitro, population, system, or assay. Exemplary metrics of disease can include, without limitation, liver weight, total cholesterol in plasma, alanine transaminase (ALT) in plasma, aspartate transaminase (AST) in plasma, total bile acids in plasma, NAFLD (nonalcoholic fatty liver disease) score, steatosis score, and/or percentage of hepatocytes with lipid droplets.

Various obesity therapies are known in the art, and can include therapies that cause a reduction in calorie intake, increase in energy expenditure, or change in appetite. In some embodiments, the term obesity therapies, as used herein, includes or refers to therapies, agents, or regimens that can reduce the total body weight of an individual, e.g., of an individual characterized as having obesity or pre-obesity, and/or are prescribed for that purpose. Obesity therapies include obesity therapeutics, which as used herein include or refer to agents (e.g., drugs) that can be used to reduce the total body weight of an individual, e.g., of an individual characterized as having obesity or pre-obesity, and/or are prescribed for that purpose.

Specific examples of obesity therapies, and in particular of obesity therapeutics, include glucagon-like peptide-1 (GLP-1) agonists. A variety of GLP-1 agonists are known in the art and include, for example, semaglutide, liraglutide, dulaglutide, exenatide, tirzepatide, lixisenatide, and albiglutide. Semaglutide is found in products such as WEGOVY®, OZEMPIC®, and RYBELSUS®. Liraglutide is found in products such as SAXENDA® and VICTOZA®. Tirzepatide is found in products such as ZEPBOUND® and MOUNJARO®. Without wishing to be bound by any particular scientific theory, GLP-1 agonists (like semaglutide and liraglutide) mimic the natural hormone glucagon-like peptide-1 (GLP-1) and are exemplary of obesity therapeutics that are understood to promote fullness and delay stomach emptying.

In various examples of obesity therapies, a GLP-1 agonist can be used in further combination with one or more (e.g., one or two) additional agents. In some embodiments, one or more additional agents used in combination with a GLP-1 agonist can target other metabolic pathways. In some embodiments, one or more additional agents used in combination with a GLP-1 agonist can include glucagon (GCG) and/or a glucose-dependent insulinotropic polypeptide (GIP). For example, tirzepatide is a dual therapy that includes a glucose-dependent insulinotropic polypeptide (GIP) receptor agonist and a glucagon-like peptide-1 (GLP-1) receptor agonist. Retatrutide is a triple therapy that includes a glucose-dependent insulinotropic polypeptide (GIP) receptor agonist, a glucagon receptor agonist, and a glucagon-like peptide-1 (GLP-1) receptor agonist

Other examples of obesity therapeutics include orlistat, phentermine, topiramate, phentermine-topiramate, naltrexone, bupropion, naltrexone-bupropion, and setmelanotide. Without wishing to be bound by any particular scientific theory, orlistat is an obesity therapeutic thought to block fat absorption, phentermine-topiramate is an obesity therapeutic thought to suppress appetite (phentermine) and increase feelings of fullness (topiramate), naltrexone-bupropion is an obesity therapeutic thought to suppress appetite and food reward pathways, and setmelanotide is a melanocortin-4 (MC4) receptor agonist thought to reduce appetite. Orlistat is found in products such as XENICAL® and ALLI®. Phentermine is found in products such as ADIPEX®, SUPRENZA®, and LOMAIRA®. Phentermine-topiramate is found in products such as QSYMIA®. Naltrexone-bupropion is found in products such as CONTRAVER.

Other examples of obesity therapeutics include surgical interventions and medical devices. Exemplary surgical interventions include bariatric and metabolic surgeries, such as gastric bypass surgery, biliopancreatic diversion with duodenal switch (BPD/DS) surgery, and sleeve gastrectomy. Devices used in treatment of obesity can include, e.g., intragastric ballons, endoscopic suturing devices, vagal nerve stimulators, endoluminal bypass liners, hydrogel particles (e.g., a biodegradable, super-absorbent hydrogel released into the stomach such as in the in the oral superabsorbent hydrogel product PLENITY®), and others. Thus, in various embodiments, an OSK therapy of the present disclosure can be administered to a subject in a course of treatment that further includes administration of one or more additional therapies that are not therapeutic agents (e.g., surgery).

Various therapies for liver disease, and in particular therapies for MASH, are known in the art. Therapies for treatment of liver disease, and in particular for treatment of MASH, include agents that activate the liver's thyroid hormone receptor-beta (THR-β), reduce fibrogenesis, reduce liver steatosis, increase insulin sensitivity, reduce dyslipidemia, reduce triglyceride levels, and reduce liver cholesterol. Therapies for treatment of liver disease, and in particular for treatment of MASH, include agents that reduce one or more of liver weight, total cholesterol in plasma, alanine transaminase (ALT) in plasma, aspartate transaminase (AST) in plasma, total bile acids in plasma, NAFLD (nonalcoholic fatty liver disease) score, steatosis score, and/or percentage of hepatocytes with lipid droplets. Therapies for treatment of liver disease, and in particular for treatment of MASH, include, e.g., the therapeutic agent resmetirom, PPAR agonists, FGF21 analogs, and vitamin E.

Without wishing to be bound by any particular scientific theory, resmetirom functions as a THR-β agonist, specifically targeting liver function to achieve what can be described as “localized hyperthyroidism” within the liver. This targeted action stimulates lipid metabolism and reduces lipid accumulation and liver fibrosis. Resmetirom is found in the product REZDIFFRA®.

Without wishing to be bound by any particular scientific theory, PPAR agonists can modulate one or more of lipid metabolism, insulin sensitivity, and fibrosis progression. Example of PPAR agonists include lanifibranor (a pan-PPAR agonist) and saroglitazar (a PPAR-γ/α dual agonist).

Without wishing to be bound by any particular scientific theory, FGF21 analogs can modulate hepatic lipid metabolism, glucose homeostasis, and fibrosis progression. Examples of FGF21 analogs include efruxifermin and pegbelfermin.

The present disclosure includes the recognition that obesity therapy and a liver disease therapy can contribute to treatment of liver disease and/or MASH by mechanisms that are distinct from that of OSK therapy, and can therefore be additive, synergistic, and/or adjunctive with OSK therapy for treatment thereof.

In accordance with the above, exemplary combination therapies of the present disclosure can include, without limitation, an OSK therapy in combination with an obesity therapy, such as an OSK therapy in combination with a GLP-1 agonist (such as semaglutide, liraglutide, dulaglutide, exenatide, tirzepatide, lixisenatide, or albiglutide), an OSK therapy in combination with an obesity therapeutic such as orlistat, phentermine, topiramate, phentermine-topiramate, naltrexone, bupropion, naltrexone-bupropion, or setmelanotide, or an OSK therapy in combination with a surgery or device for treatment of obesity. In some embodiments, the present disclosure includes an OSK therapy in combination with semaglutide. In some embodiments, the present disclosure includes an OSK therapy in combination with liraglutide. In some embodiments, the present disclosure includes an OSK therapy in combination with dulaglutide. In some embodiments, the present disclosure includes an OSK therapy in combination with exenatide. In some embodiments, the present disclosure includes an OSK therapy in combination with tirzepatide. In some embodiments, the present disclosure includes an OSK therapy in combination with lixisenatide. In some embodiments, the present disclosure includes an OSK therapy in combination with albiglutide. In some embodiments, the present disclosure includes an OSK therapy in combination with a GLP-1 agonist and a further therapeutic agent. In some embodiments, the present disclosure includes an OSK therapy in combination with a GLP-1 agonist and glucagon. In some embodiments, the present disclosure includes an OSK therapy in combination with a GLP-1 agonist and a glucose-dependent insulinotropic polypeptide. In some embodiments, the present disclosure includes an OSK therapy in combination with a GLP-1 agonist, glucagon, and a glucose-dependent insulinotropic polypeptide.

In accordance with the above, exemplary combination therapies of the present disclosure can include, without limitation, an OSK therapy in combination with a liver disease therapy (e.g., a MASH therapy), such as an OSK therapy in combination with a THR-β agonist, an OSK therapy in combination with a PPAR agonist, an OSK therapy in combination with an FGF21 analog, or an OSK therapy in combination with vitamin E. In some embodiments, the present disclosure includes an OSK therapy in combination with resmetirom. In some embodiments, the present disclosure includes an OSK therapy in combination with lanifibranor and saroglitazar. In some embodiments, the present disclosure includes an OSK therapy in combination with efruxifermin and pegbelfermin.

Combination therapies of the present disclosure can include simultaneous exposure of a subject to two or more therapies, agents, or regimens, e.g., to pharmaceutical agents of two or more therapeutic regimens.

In certain embodiments, an OSK therapy as described herein can be administered together with (e.g., at the same time and/or in the same composition as) an additional therapy, agent, or regimen. In certain embodiments, a therapeutic agent of the present disclosure can be administered separately from an additional therapy, agent, or regimen (e.g., at a different time and/or in a different composition than the additional therapy, agent, or regimen). Dosing regimens of an OSK therapy and of one or more additional agents with which it is administered in combination can be coordinated or independently determined. In various embodiments, an additional therapy, agent, or regimen administered in combination with an OSK therapy described herein can be administered at the same time as the OSK therapy, on the same day as the OSK therapy, or in the same week as the OSK therapy. In various embodiments, an additional agent or therapy administered in combination with an OSK therapy as described herein can be administered such that administration of the OSK therapy the additional agent or therapy are separated by one or more hours before or after, one or more days before or after, one or more weeks before or after, or one or more months before or after administration of the OSK therapy. In various embodiments, the administration frequency and/or dosage of one or more additional agents can be the same as, similar to, or different from the administration of the OSK therapy. In some embodiments, an OSK therapy and an additional therapy, agent, or regimen can be administered simultaneously, sequentially, or overlappingly.

In certain embodiments, administration of an OSK therapy can be to a subject having previously received, scheduled to receive, or in the course of a treatment regimen including an additional therapy, agent, or regimen. In some embodiments, administration of an additional therapy, agent, or regimen can improve delivery or efficacy of an OSK therapy with which it is administered in combination. In some embodiments, administration of an OSK therapy can improve delivery or efficacy of an additional therapy, agent, or regimen with which it is administered in combination.

It is contemplated that therapeutic agent combination therapies can demonstrate additive effects, adjunctive effects, and/or greater-than-additive or synergistic effects between an OSK therapy and one or more additional therapeutic therapies, agents, or regimens with which it is administered in combination. An OSK therapy can be administered in any effective amount as determined independently or as determined by the joint action of the OSK therapy and any of one or more additional therapies, agents, or regimens administered. Administration of the OSK therapy may, in some embodiments, reduce the therapeutically effective dosage, required dosage, or administered dosage of the additional therapies, agents, or regimens relative to a reference regimen for administration of the additional therapies, agents, or regimens in the absence the therapeutic agent. In certain embodiment, a composition described herein can replace or augment other previously or currently administered therapies, agents, or regimens. For example, upon treating with OSK therapy, administration of one or more additional therapies, agents, or regimens can cease or diminish, e.g., be administered at lower levels.

### Effects

Disclosed herein, in some embodiments, are methods that include treating a subject or administering a composition. The administration may have an effect, such as improvement of a symptom of a liver disease or a reversal of epigenetic change. For example, the treatment may improve a liver steatosis, fibrosis, or inflammation measurement in a subject. The improvement may be relative to a baseline measurement. In some embodiments, the administration rejuvenates liver cells in the subject. In some embodiments, the liver cells comprise hepatocytes or stellate cells (HSCs). An effect may include a partial reversal of epigenetic change. An effect may include a complete reversal of epigenetic change. An effect may include reversion of a disease (e.g. liver disease) to an earlier state of disease.

In some embodiments, the induces expression of partial reprogramming factors (e.g. OCT4, SOX2, and KLF4) in the subject. In some embodiments, the composition does not induce c-Myc expression in the subject. In some embodiments, the composition does not reprogram a cell, tissue, or organ to a pluripotent state in the subject.

Some embodiments include obtaining a reprogramming factor measurement of a subject. The reprogramming factor measurement may be obtained following administration of a composition herein such as an expression vector encoding partial reprogramming factors. Some embodiments include obtaining a baseline reprogramming factor measurement. The baseline reprogramming factor measurement may be obtained before the administration. The reprogramming factor measurement may be or include an intracellular reprogramming factor measurement. The reprogramming factor measurement may be obtained from a blood or tissue sample.

Administration of a composition herein (e.g. an AAV-OSK or LNP-OSK) may increase expression of OSK proteins. In some embodiments, the administration increases a reprogramming factor measurement such as an OCT4 measurement, a SOX2 measurement, a KLF4 measurement, or a combination thereof in the subject. A reprogramming factor may be OCT4, SOX2, or KLF4. The OCT4 measurement may include an OCT4 protein or RNA measurement. The reprogramming factor measurement may include a SOX2 measurement. The SOX2 measurement may include a SOX2 protein or RNA measurement. The reprogramming factor measurement may include a KLF4 measurement. The KLF4 measurement may include a KLF4 protein or RNA measurement. An RNA measurement may be or include an mRNA measurement. The reprogramming factor measurement may include a combination of reprogramming factor measurement (e.g. a measurement of OCT4, SOX2, and KLF4 protein or mRNA levels).

In some embodiments, the administration increases a reprogramming factor measurement (e.g. a measurement of OCT4, SOX2, or KLF4 protein) in the subject. In some embodiments, the administration increases the reprogramming factor measurement relative to the baseline reprogramming factor measurement. In some embodiments, the reprogramming factor measurement is increased by about 2.5% or more, about 5% or more, or about 7.5% or more, relative to the baseline reprogramming factor measurement. In some embodiments, the reprogramming factor measurement is increased by at least 5% relative to a baseline measurement. In some embodiments, the reprogramming factor measurement is increased by about 10% or more, relative to the baseline reprogramming factor measurement. Some embodiments include performing an assay to determine the increased reprogramming factor following administration a composition (e.g. following administration of an expression vector or one or more nucleotides encoding partial reprogramming factors).

In some embodiments, a reprogramming factor measurement may include a c-Myc measurement such as a c-Myc protein or RNA measurement. In some embodiments, the reprogramming factor measurement is not affected by an administration. In some embodiments, the reprogramming factor measurement (e.g. c-Myc measurement) is increased by no more than about 2.5%, no more than about 5%, or no more than about 7.5%, relative to the baseline reprogramming factor measurement (e.g. baseline c-Myc measurement). In some embodiments, the reprogramming factor measurement (e.g. c-Myc measurement) is increased by no more than about 10%, relative to the baseline reprogramming factor measurement (e.g. baseline c-Myc measurement). Some embodiments include performing an assay to determine an effect or lack of effect on a reprogramming factor measurement following treatment or administration. In some embodiments, there is a small or insignificant effect on a c-Myc measurement upon treatment or after an administration of a composition.

Some embodiments include obtaining liver steatosis measurement of a subject. The liver steatosis measurement may be obtained following administration of a composition herein, such as an expression vector or one or more nucleotides encoding partial reprogramming factors. Some embodiments include obtaining a baseline liver steatosis measurement of a subject. The baseline liver steatosis measurement may be obtained before the administration. The liver steatosis measurement may be obtained from a biopsy. The liver steatosis measurement may be obtained by medical imaging. The liver steatosis measurement may be improved relative to a baseline liver steatosis measurement.

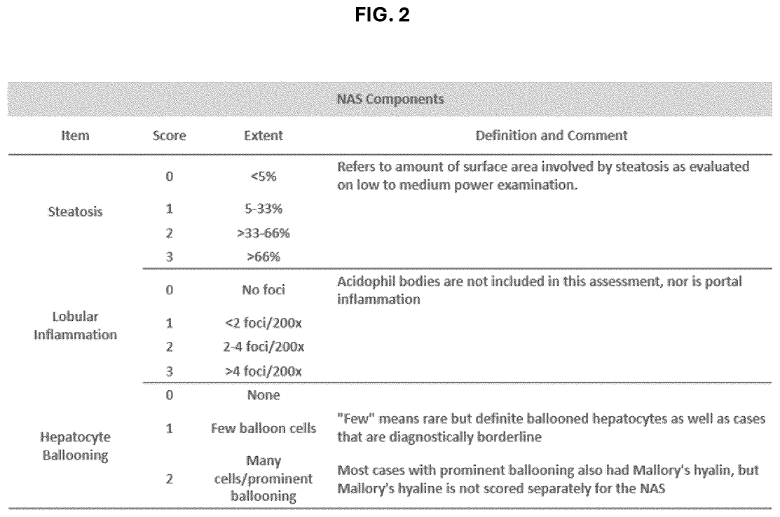

Some embodiments affect or improve a NAFLD activity score (NAS) of a subject. A NAS improvement may include a reduction in the NAS. The NAS may be determined based on any criteria in FIG. 2 (from Kleiner D. E., Brunt E. M., Van Natta M., Behlinh C., Contos M. J., Cummings O. W., Ferrell L. D., Liu Y.-C., Torbenson M. S., Unalp-Arida A., Yeh M., Mccullough A. J., Sanyal A. J. for the Nonalcoholic Steatohepatitis Clinical Research Network. Design and validation of a histological scoring system for nonalcoholic fatty liver disease. Hepatology 41:1313-1321, 2005, which is incorporated herein by reference in its entirety). In some embodiments, a NAS below 3 is indicative of a lack of MASH. In some embodiments, a NAS below 4 is indicative of a lack of MASH. In some embodiments, a NAS at least 4 or 5 is indicative of MASH.

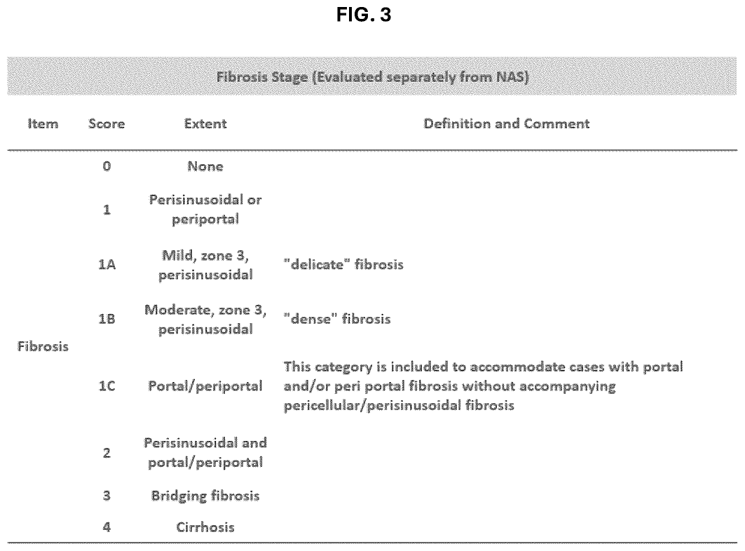

Some embodiments include obtaining liver fibrosis measurement of a subject. The liver fibrosis measurement may be obtained following administration of a composition herein, such as an expression vector or one or more nucleotides encoding partial reprogramming factors. Some embodiments include obtaining a baseline liver fibrosis measurement of a subject. The baseline liver fibrosis measurement may be obtained before the administration. The liver fibrosis measurement may be obtained from a biopsy. The liver fibrosis measurement may be obtained by medical imaging. The liver fibrosis measurement may be improved relative to a baseline liver fibrosis measurement. Some embodiments decrease DNA methylation. For example, some embodiments may decrease methylation at a CpG site associated with fibrosis. Some embodiments may result in an improvement in a MASH fibrosis stage or a MASH fibrosis score, for example as determined based on any criteria in FIG. 3 (from Honda Y, Yoneda M, Imajo K, Nakajima A. Elastography Techniques for the Assessment of Liver Fibrosis in Non-Alcoholic Fatty Liver Disease. International Journal of Molecular Sciences. 2020; 21 (11): 4039, which is incorporated herein by reference in its entirety).

Some embodiments include obtaining liver inflammation measurement of a subject. The liver inflammation measurement may be obtained following administration of a composition herein, such as an expression vector or one or more nucleotides encoding partial reprogramming factors. Some embodiments include obtaining a baseline liver inflammation measurement of a subject. The baseline liver inflammation measurement may be obtained before the administration. The liver inflammation measurement may be obtained from a biopsy. The liver inflammation measurement may be obtained by medical imaging. The liver inflammation measurement may be improved relative to a baseline liver inflammation measurement.

Some embodiments include obtaining liver disease biomarker measurement of a subject. The liver disease biomarker measurement may be obtained following administration of a composition herein, such as an expression vector or one or more nucleotides encoding partial reprogramming factors. Some embodiments include obtaining a baseline liver disease biomarker measurement of a subject. The baseline liver disease biomarker measurement may be obtained before the administration. The liver disease biomarker measurement may be obtained in a biofluid sample. The liver disease biomarker measurement may be obtained in a blood, serum, or plasma sample. The liver disease biomarker measurement may be obtained in a tissue sample. The liver disease biomarker measurement may be obtained from a biopsy. The liver disease biomarker measurement may be improved relative to a baseline liver disease biomarker measurement.

In some embodiments, treatment can be measured as a decrease in ALT in plasma in a subject. In some embodiments, treatment can be measured as a decrease in AST in plasma in a subject. In some embodiments, treatment can be measured as a decrease in total bile acids in plasma in a subject. In some embodiments, treatment can be measured as a decrease in total cholesterol in plasma in a subject. In some embodiments, treatment can be measured as an improvement of at least 1 point in NAFLD score in a subject. In some embodiments, treatment can be measured as a decrease in the percentage of hepatocytes with lipid droplets in a subject. In some embodiments, treatment can be measured as a decrease in liver weight in a subject. In some embodiments, treatment can be measured as an improvement of at least 1 point in steatosis score in a subject. In some embodiments, OSK therapy does not cause a decrease in body weight of a subject.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining.” “measuring,” “evaluating,” “assessing.” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

A liver disease may include metabolic dysfunction-associated steatotic liver disease (MASLD) and metabolic dysfunction-associated steatohepatitis (MASH). In some embodiments, a liver disease such as MASLD can be diagnosed using criteria found at Rinella M E, Lazarus J V, Ratziul V, et al. A multi-society Delphi consensus statement on new fatty liver disease nomenclature. Hepatology. Published online Jun. 24, 2023, which is incorporated herein by reference in its entirety. MASLD may include or progress to MASH. Some aspects relating to progression of MASLD are provided at Karim G, Bansal M B. Resmetirom: An Orally Administered, Small-molecule, Liver-directed, β-selective THR Agonist for the Treatment of Non-alcoholic Fatty Liver Disease and Non-alcoholic Steatohepatitis. touchREV Endocrinol. 2023; 19 (1): 60-70, which is incorporated herein by reference in its entirety.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

A subject to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals, such as cattle, pigs, horses, sheep, goats, cats, and/or dogs) and birds (e.g., commercially relevant birds, such as chickens, ducks, geese, and/or turkeys). In certain embodiments, the animal is a mammal. The animal may be a male or female and at any stage of development. A non-human animal may be a transgenic animal.

As used herein, the term “about” a number refers to that number plus or minus 15% of that number. The term “about” a range refers to that range minus 15% of its lowest value and plus 15% of its greatest value.

As used herein, the terms “treatment,” “treat,” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

A treatment may include reversing, alleviating, delaying the onset of, or inhibiting the progress of a disease or disorder, or one or more symptoms thereof, as described herein. In certain embodiments, treatment may be administered after one or more symptoms have developed. In other embodiments, treatment may be administered in the absence of symptoms. For example, treatment may be administered to a susceptible individual prior to the onset of symptoms. Treatment may also be continued after symptoms have resolved, for example, to prevent or delay their recurrence.

The section headings used herein are for organizational purposes only and are not to be construed as limiting the subject matter described.

“AAV” or “adeno-associated virus” is a nonenveloped virus that is capable of carrying and delivering nucleic acids (e.g., engineered nucleic acids encoding OCT4; KLF4; SOX2; or any combination thereof) and belongs to the genus Dependoparvovirus. In some instances, an AAV is capable of delivering a nucleic acid encoding an inducing agent. In general, AAV does not integrate into the genome. The tissue-specific targeting capabilities of AAV is often determined by the AAV capsid serotype (see, e.g., Table 1 for examples of AAV serotypes and their utility in tissue-specific delivery). Non-limiting serotypes of AAV include AAV1, AAV2, AAV3, AAV4, AAV5, AAV6, AAV7, AAV8, AAV9, AAV 10, AAV11, and variants thereof. In certain embodiments, the AAV serotype is a variant of AAV9 (e.g., AAV PHP.b). An AAV serotype identified as relevant to liver tissue may be useful as a delivery vehicle to liver.

TABLE 1
Non-limiting Examples of AAV Serotypes and their Use in Specific Tissues

| | Relevant Tissue | | | | | | | | |
|---|---|---|---|---|---|---|---|---|---|
| | | | | | | | | | Immune |
| | | | | | | Central | | | System |
| | | | | | | Nervous | | | (T-cells, |
| | | | Muscle | | Central | System | | | B-cells |
| | | | (e.g., | | Nervous | (Blood- | | | and |
| AAV | | | Skeletal | | System | brain | | | Dendritic |
| Serotype | Liver | Heart | Muscle) | Eye | (CNS) | barrier) | Pancreas | Lung | Cells) |
| AAV1 | | X | X | | X | | | | |
| AAV2 | X | | X | X | X | | | | |
| AAV3 | X | | | | | | | X | |
| AAV4 | | | | X | X | | | | |
| AAV5 | | | X | X | X | | X | X | |
| AAV6 | X | X | X | | | | | X | X |
| (e.g., | | | | | | | | | |
| AAV6.2) | | | | | | | | | |
| AAV7 | X | | X | | | | | | |
| AAV8 | X | X | X | | X | | X | | |
| AAV9 | X | X | X | X | X | X | X | X | |
| AAV10 | X | X | X | X | X | X | X | X | |
| (e.g., | | | | | | | | | |
| AAVrh10) | | | | | | | | | |
| AAVDJ | X | | X | X | X | | | | |
| AAVPHP.B | | | | | X | X | | | |

A “recombinant virus” is a virus (e.g., lentivirus, adenovirus, retrovirus, herpes virus, alphavirus, vaccinia virus or adeno-associated virus (AAV)) that has been isolated from its natural environment (e.g., from a host cell, tissue, or a subject) or is artificially produced.

The term “AAV vector” as used herein is a nucleic acid that comprises AAV inverted terminal repeats (ITRs) flanking an expression cassette (e.g., an expression cassette comprising a nucleic acid encoding OCT4, KLF4, and SOX2, each alone or in combination, or an expression cassette encoding rtTA or tTA). An AAV vector may further comprise a promoter sequence.

The terms “administer,” “administering,” or “administration,” as used herein refers to introduction of any of the compositions described herein; any of the nucleic acids capable of inducing OCT4, KLF4, and/or SOX2 expression; any of the nucleic acids capable of inducing expression of one or more transcription factors selected from the group consisting of OCT4, KLF4, SOX2, and any combinations thereof; any of the engineered proteins described herein; any of the chemical agents activating (e.g., inducing expression of) OCT4, KLF4, and/or SOX2; any of the chemical agents activating (e.g., inducing expression of) one or more transcription factors selected from OCT4, KLF4, SOX2, and any combinations thereof; any of the antibodies activating (e.g., inducing expression of) OCT4, KLF4, and/or SOX2, and any combinations thereof; and/or any of the recombinant viruses (e.g., lentivirus, adenovirus, alphavirus, vaccinia virus, retrovirus, herpes virus, or AAV) described herein, alone, or in combination to any cell, tissue, organ, and/or subject. In some embodiments, a nucleic acid encoding an inducing agent, an engineered protein encoding an inducing agent, a chemical agent capable of modulating (e.g., activating or inhibiting) the activity of an inducing agent, and/or a recombinant virus encoding an inducing agent is also administered to the cell, tissue, organ and/or subject. Any of the compositions described herein, comprising any of the nucleic acids capable of inducing expression of one or more transcription factors selected from OCT4, KLF4, SOX2, and any combinations thereof; any of the chemical agents activating (e.g., inducing expression of, e.g., tetracyline) OCT4, KLF4, and/or SOX2; any of the engineered proteins encoding OCT4, SOX2, KLF4, or any combinations thereof; any of the chemical agents activating (e.g., inducing expression of, e.g., tetracyline) OCT4, KLF4, SOX2, or any combination thereof; any of the antibodies activating (e.g., inducing expression of) OCT4, KLF4, and/or SOX2; and/or any of the recombinant viruses (e.g., lentivirus, adenovirus, alphavirus, vaccinia virus, retrovirus, herpes virus, or AAV) described herein, alone, or in combination may be administered as described herein, such as intravenously or by injection.

As used herein, the term “cell” is meant not only to include an individual cell but refers also to the particular tissue or organ from which it originates.

The term “gene expression” refers to the degree to which certain genes or all genes in a cell or tissue are transcribed into RNA. In some instances, the RNA is translated by the cell into a protein. The epigenome dictates gene expression patterns.

The terms “condition,” “disease,” and “disorder” may be used interchangeably. Metabolic dysfunction-associated steatotic liver disease (MASLD) may be used interchangeably with non-alcoholic fatty liver disease (NAFLD). Non-alcoholic steatohepatitis (NASH) may be used interchangeably with metabolic dysfunction-associated steatohepatitis (MASH).

Cellular causes of aging may include loss or modification of epigenetic information.

The terms, “MYC proto-oncogene,” “c-Myc,” or “Myc.” refer to a nuclear phosphoprotein that has been implicated in cell cycle progression. c-Myc is capable of forming a heterodimer with the transcription factor MAX and the heterodimer is capable of binding to an E box consequence sequence on nucleic acids (e.g., engineered nucleic acids) to regulate transcription of target genes. In certain embodiments, a nucleotide sequence encoding c-Myc comprises a sequence that is at least 70% (e.g., at least 75%, 80%, 85%, 90%, 95%, 98%, 99%, or 100%) identical to a sequence as described in the NCBI RefSeq database under accession number NM_001354870.1 or NM_002467.5. In certain embodiments, an amino acid sequence encoding c-Myc comprises a sequence that is at least 70% (e.g., at least 75%, 80%, 85%, 90%, 95%, 98%, 99%, or 100%) identical to NP 002458.2 or NP 001341799.1. In certain embodiments, the methods comprise inducing expression of OCT4; KLF4; SOX2; or any combination thereof in the absence of inducing c-Myc expression or in the absence of activating c-Myc. Absence of inducing c-Myc expression may refer to absence of substantial induction of c-Myc expression over endogenous levels of c-Myc expression in a cell, tissue, subject, or any combination thereof. Absence of substantial induction of c-Myc expression as compared to endogenous levels of c-Myc expression in a cell, tissue, subject, or any combination thereof, may refer to increasing c-Myc expression by less than 70%, less than 60%, less than 50%, less than 40%, less than 30%, less than 20%, less than 10%, or any values in between as compared to endogenous levels of c-Myc expression in the cell, tissue, subject, or any combination thereof. Absence of activating c-Myc expression may refer to absence of substantial activation of c-Myc (e.g., activity) over endogenous c-Myc activity in a cell, tissue, subject, or any combination thereof. Absence of substantial induction of c-Myc activity as compared to endogenous c-Myc activity in a cell, tissue, subject, or any combination thereof, may refer to increasing c-Myc activity by less than 70%, less than 60%, less than 50%, less than 40%, less than 30%, less than 20%, less than 10%, or any values in between as compared to endogenous c-Myc activity in the cell, tissue, subject, or any combination thereof.

The terms “effective amount” and “therapeutically effective amount,” as used herein, may refer to the amount of a compound or composition, that, when administered to a subject, is effective to at least partially treat a condition from which the subject is suffering.

As used herein, a protein that is “functional” or “active” may be one that retains its biological activity (e.g., capable of acting as a transcription factor or as an inducing agent). Conversely, a protein that is not functional or is inactive may be one that is not capable of performing one or more of its wild-type functions.

The term “gene” refers to a nucleic acid fragment that expresses a protein, including regulatory sequences preceding (5 ‘non-coding sequences) and following (3’ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

“Homolog” or “homologous” refers to sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity). Homologous sequences include but are not limited to paralogous or orthologous sequences. Paralogous sequences arise from duplication of a gene within a genome of a species, while orthologous sequences diverge after a speciation event. A functional homolog retains one or more biological activities of a wild-type protein. In certain embodiments, a functional homolog of OCT4, KLF4, or SOX2 retains at least 10%, at least 20%, at least 30%, at least 40%, at least 50%, at least 60%, at least 70%, at least 80%, at least 90%, or at least 100% of the biological activity (e.g., transcription factor activity) of a wild-type counterpart.

“KLF4” may also be referred to as Krüppel-like factor 4, EZF, or GKLF and is a zinc-finger transcription factor. KLF4 has been implicated in regulation of differentiation and proliferation and is capable of interacting with co-activators, including members of the p300-CBP coactivator family. A KLF4 transcription factor, homolog (e.g., functional homolog), or variant thereof, as used herein, may be derived from any species, including humans. In certain embodiments, the nucleic acid encoding human KLF4 comprises a sequence that is at least 70% (e.g., at least 75%, 80%, 85%, 90%, 95%, 98%, 99%, or 100%) identical to a nucleic acid described in the NCBI RefSeq database under accession number NM_004235.5 or NM_001314052.1. Non-limiting examples of KLF4 variants include Kruppel-like factor 4 transcript variant 1 and Kruppel-like factor 4 transcript variant 2. In certain embodiments, KLF4 is encoded by a nucleic acid molecule comprising a nucleic acid sequence that is at least 70% (e.g., at least 75%, 80%, 85%, 90%, 95%, 98%, 99%, or 100%) identical to SEQ ID NO: 5. SEQ ID NO: 5 is a non-limiting example of a nucleotide sequence encoding human KLF4. In certain embodiments, KLF4 comprises an amino acid sequence that is at least 70% (e.g., at least 75%, 80%, 85%, 90%, 95%, 98%, 99%, or 100%) identical to NP_001300981.1 or NP_004226.3. In certain embodiments, KLF4 comprises an amino acid sequence that is at least 70% (e.g., at least 75%, 80%, 85%, 90%, 95%, 98%, 99%, or 100%) identical to SEQ ID NO: 6. SEQ ID NO: 6 is a non-limiting example of an amino acid sequence of human KLF4.

“Inverted terminal repeats” or “ITRs” are nucleic acid sequences that are reverse complements of one another. In general, in an AAV vector, ITRs are found on either side of a cassette (e.g., an expression cassette comprising a nucleic acid encoding OCT4; KLF4; SOX2; or any combination thereof). For example, the ITRs flanking the OSK cassette may comprise SEQ ID NOs: 16 and 32. Similarly, in some instances, the AAV2-CMV-rtTA3 vector disclosed herein can include ITRs comprising SEQ ID NOs: 22 and 33, and the AAV2-CMV-rtTA4 vector disclosed herein can include ITRs comprising SEQ ID NOs: 29 and 34. In some instances, the cassette encodes an inducing agent. AAV ITRs include ITRs from AAV1, AAV2, AAV3, AAV4, AAV5, AAV6, AAV7, AAV8, AAV9, AAV 10, AAV11, and AAV variants thereof.

The terms “nucleic acid.” “polynucleotide”, “nucleotide sequence”, “nucleic acid molecule”, “nucleic acid sequence”, and “oligonucleotide” refer to a series of nucleotide bases (also called “nucleotides”) in DNA and RNA, and mean any chain of two or more nucleotides. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids.

Some nucleic acids described herein may be synthesized by standard methods known in the art, e.g., by use of an automated DNA synthesizer (such as those that are commercially available from Biosearch, Applied Biosystems, etc.). Such DNA sequences may be incorporated into a wide variety of vectors that incorporate suitable RNA polymerase promoters such as the T7 or SP6 polymerase promoters. A vector can be introduced in vivo such that it is taken up by a cell and directs the transcription of the nucleic acid molecule. Such a vector can remain episomal or become chromosomally integrated, as long as it can be transcribed. Such vectors can be constructed by recombinant DNA technology methods standard in the art. Vectors can be plasmid, viral, or others known in the art, used for replication and expression in mammalian cells. Expression of the sequence can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be administered to the subject.

Some nucleic acid molecules may include natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5″- and 3″-non-coding regions, and the like. A “recombinant nucleic acid molecule” or “engineered nucleic acid molecule” is a nucleic acid molecule that has undergone a molecular biological manipulation, i.e., non-naturally occurring nucleic acid molecule or genetically engineered nucleic acid molecule. Furthermore, the terms “recombinant DNA molecule” or “engineered nucleic acid” refer to a nucleic acid sequence which is not naturally occurring, or can be made by the artificial combination of two otherwise separated segments of nucleic acid sequence, i.e., by ligating together pieces of DNA that are not normally contiguous. By “recombinantly produced” is meant artificial combination often accomplished by either chemical synthesis means, or by the artificial manipulation of isolated segments of nucleic acids, e.g., by genetic engineering techniques using restriction enzymes, ligases, and similar recombinant techniques as described by, for example, Sambrook et al, Molecular Cloning, second edition, Cold Spring Harbor Laboratory, Plainview, N.Y.: (1989), or Ausubel et al, Current Protocols in Molecular Biology, Current Protocols (1989), and DNA Cloning: A Practical Approach, Volumes I and II (ed. D. N. Glover) IREL Press, Oxford, (1985); each of which is incorporated herein by reference.

Such manipulation may be done to replace a codon with a redundant codon encoding the same or a conservative amino acid, while typically introducing or removing a sequence recognition site. Alternatively, it may be performed to join together nucleic acid segments of desired functions to generate a single genetic entity comprising a desired combination of functions not found in nature. Restriction enzyme recognition sites are often the target of such artificial manipulations, but other site-specific targets, e.g., promoters, DNA replication sites, regulation sequences, control sequences, open reading frames, or other useful features may be incorporated by design.

“OCT4” may also be referred to as octamer-binding transcription factor 4, OCT3, OCT3/4, POU5F1, or POU class 5 homeobox 1 and is a transcription factor that has been implicated in embryonic development and determination of cell fate. Similar to other OCT transcription factors, OCT4 is characterized by a bipartite DNA binding domain called a POU domain. An OCT4 transcription factor, homolog, or variant thereof, as used herein, may be derived from any species, including humans. In certain embodiments, the nucleic acid encoding human OCT4 is at least 70% (e.g., at least 75%, 80%, 85%, 90%, 95%, 98%, 99%, or 100%) identical to a nucleic acid described in the NCBI RefSeq under accession number NM_002701, NM_203289, NM_001 173531, NM_001285986, or NM_001285987. In certain embodiments, the nucleic acid molecule encoding a human OCT4 comprises a nucleic acid sequence that is at least 70% (e.g., at least 75%, 80%, 85%, 90%, 95%, 98%, 99%, or 100%) identical to a nucleic acid sequence provided as SEQ ID NO: 1. SEQ ID NO: 1 is a non-limiting example of a nucleotide sequence encoding human OCT4. Non-limiting examples of OCT4 variants encompassed herein include POU5F1, transcript variant 1, POU5F1, transcript variant 2, POU5F1, transcript variant 3, POU5F1, transcript variant 4, and POU5F1 transcript variant 5. In certain embodiments, the nucleic acid molecule encodes an OCT4 comprising an amino acid sequence at least 70% (e.g., at least 75%, 80%, 85%, 90%, 95%, 98%, 99%, or 100%) identical to an amino acid sequence described in the NCBI RefSeq under accession number NP 001167002.1. NP_001272915.1, NP_001272916.1, NPJ302692.2, or NP_976034.4. In certain embodiments, the nucleic acid molecule encodes an OCT4 comprising an amino acid sequence that is at least 70% (e.g., at least 75%, 80%, 85%, 90%, 95%, 98%, 99%, or 100%) identical to SEQ ID NO: 2. SEQ ID NO: 2 is a non-limiting example of an amino acid sequence of human OCT4. Other OCT4 transcription factors (e.g., from other species) are known and nucleic acids encoding OCT4 transcription factors can be found in publicly available databases, including GenBank.

The term “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific, or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation of that sequence, expression of that sequence, or a combination thereof.

A promoter may promote ubiquitous expression or tissue-specific expression of an operably linked nucleic acid sequence from any species, including humans. In some embodiments, the promoter is a eukaryotic promoter. Non limiting examples of eukaryotic promoters may include TDH3, PGK1, PKC1, TDH2, PYK1, TPI1, ATI, CMV, EF1 alpha, SV40, PGK1 (human or mouse), Ubc, human beta actin, CAG, TRE, UAS, Ac5, Polyhedrin, CaMKIIa, GALI, GAL10, TEF1, GDS, ADHI, CaMV35S, Ubi, Hl, or U6. In some embodiments, a promoter of the present disclosure is suitable for use in AAV vectors. In certain embodiments, the promoter is a mammalian promoter. In certain embodiments, the promoter is a human promoter. A tissue-specific promoter may include a liver-specific promoter. The promoter may be recombinant (e.g. a recombinant liver-specific promoter).

Some embodiments relate to or include a liver-specific promoter. The liver-specific promoter may include a human liver-specific promoter. A liver-specific promoter may include a promoter of a liver secreted protein such as human serum albumin or alpha-1-antitrypsin. A liver-specific promoter may be recombinant version of a liver-specific promoter. The recombinant promoter may be chimeric.

Some embodiments include a recombinant chimeric promoter. An example of a chimeric promoter may include a combination of an apolipoprotein E/C-I hepatic control region combined with a human alpha-1-antitrypsin core promoter. Some embodiments of a recombinant promoter include two copies of alpha 1 microglobulin/bikunin enhancer coupled to a core promoter of human thyroxine-binding globulin (TBG). Expression may be further stabilized by inclusion of a woodchuck hepatitis virus post-transcriptional regulatory element (WPRE). Some embodiments of a recombinant promoter include randomly assembled hepatocyte-specific transcription factor binding sites linked to a murine promoter such as a murine transthyretin promoter.

Non-limiting examples of ubiquitous promoters may include tetracycline-responsive promoters (under the relevant conditions), CMV (e.g., SEQ ID NO: 17), EF1 alpha, a SV40 promoter, PGK1, Ubc. CAG, human beta actin gene promoter, a RSV promoter, an EFS promoter, and a promoter comprising an upstream activating sequence (UAS).

Non-limiting examples of constitutive promoters include CP1, CMV, EF1 alpha, SV40, PGK1, Ubc, human beta actin, beta tubulin, CAG, Ac5, Rosa26 promoter, COLIA1 promoter, polyhedrin, TEF1, GDS, CaM3 5S, Ubi, Hl, U6, red opsin promoter (red promoter), rhodopsin promoter (rho promoter), cone arrestin promoter (car promoter), rhodopsin kinase promoter (rk promoter). In some instances, the constitutive promoter is a Rosa26 promoter. In some instances, the constitutive promoter is a COL1A1 promoter. A tissue-specific promoter may be used to drive expression of an engineered nucleic acid, including e.g., a nucleic acid encoding a rtTA, tTA, OCT4, KLF4, SOX2, or any combination thereof. In some embodiments, a tissue-specific promoter is used to drive expression of artTA or a rTA. In some embodiments, a tissue-specific promoter is used to drive expression of OCT4, KLF4, and SOX2. In some embodiments, the SV40 promoter is used to drive expression of OCT4, KLF4, and SOX2.

“Tetracycline” refers to the tetracycline class of antibiotic compounds that includes, but is not limited to, tetracycline, chlortetracycline, oxytetracycline, demeclocyline, lymecycline, meclocycline, methacycline, minocycline, rolitetracycline, doxycycline, tigecycline, eravacycline, sarecycline, and omadacycline.

An “inducible promoter” is one that is characterized by initiating or enhancing transcriptional activity when in the presence of, influenced by, or contacted by an inducing agent. An inducing agent may be endogenous or a normally exogenous condition, compound, agent, or protein that contacts an engineered nucleic acid in such a way as to be active in inducing transcriptional activity from the inducible promoter. In certain embodiments, an inducing agent is a tetracycline-sensitive protein (e.g., tTA or rtTA. TetR family regulators).

Inducible promoters for use in accordance with the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline responsive promoter systems, which include a tetracycline repressor protein (TetR, or TetRKRAB), a tetracycline operator sequence (tetO) and a tetracycline transactivator fusion protein ((TA), and a tetracycline operator sequence (tetO) and a reverse tetracycline transactivator fusion protein (rtTA)), steroid-regulated promoters (e.g., promoters based on the rat glucocorticoid receptor, human estrogen receptor, moth ecdysone receptors, and promoters from the steroid/retinoid/thyroid 25 receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), pH-regulated promoters, and light-regulated promoters. A non-limiting example of an inducible system that uses a light-regulated promoter is provided in Wang et al, Nat. Methods. 2012 Feb. 12; 9 (3): 266-9.

In certain embodiments, an inducible promoter comprises a tetracycline (Tet)-responsive element. For example, an inducible promoter may be a TRE3G promoter (e.g., a TRE3G promoter that comprises a sequence that is at least 70% (e.g., at least 75%, 80%, 85%, 90%, 959%, 98%, 99%, or 100%) identical to SEQ ID NO: 7). As an example, a TRE (e.g., TRE2) promoter may comprise a nucleic acid sequence that is at least 70% (e.g., at least 75%, 80%, 85%, 90%, 95%, 98%, 99%, or 100%) identical to SEQ ID NO: 7.

Additional non-limiting examples of inducible promoters include mifepristone-responsive promoters (e.g., GAL4-Elb promoter) and coumermycin-responsive promoters. See, e.g., Zhao et al., Hum Gene Ther. 2003 Nov. 20; 14 (17): 1619-29.

A “reverse tetracycline transactivator” (“rtTA”), as used herein, is an inducing agent that binds to a TRE promoter (e.g., a TRE3G, a TRE2 promoter, or a P tight promoter) in the presence of a tetracycline (e.g., doxycycline) and is capable of driving expression of a transgene that is operably linked to the TRE promoter. rtTAs generally comprise a mutant tetracycline repressor DNA binding protein (TetR) and a transactivation domain (see, e.g., Gossen et al, Science. 1995 Jun. 23; 268 (5218): 1766-9 and any of the transactivation domains listed herein). The mutant TetR domain is capable of binding to a TRE promoter when bound to tetracycline. See, e.g., US Publ. Appl. No. 2021-0403923 A, and the International Publ. No. WO2020/069339, entitled MUTANT REVERSE TETRACYCLINE TRANSACTIVATORS FOR EXPRESSION OF GENES, each of which is herein incorporated by reference in its entirety.

“Sex determining region Y-box 2,” “SRY-box 2” or “SOX2” is a member of the SRY-related HMG-box (SOX) family of transcription factors. SOX2 has been implicated in promoting embryonic development. Members of the SOX (SRY-related HMG-box) family of transcription factors are characterized by a high mobility group 5 (HMG)-box DNA sequence. This HMG box is a DNA binding domain that is highly conserved throughout eukaryotic species. A SOX2 transcription factor, homolog or variant thereof, as used herein, may be derived from any species, including humans. In certain embodiments, the nucleic acid molecule encoding SOX2 comprises a sequence that is at least 70% (e.g., at least 75%, 80%, 85%, 90%, 95%, 98%, 99%, or 100%) identical to a nucleic acid described in the NCBI RefSeq under accession number NM_01 1443.4. In certain embodiments, the nucleic acid molecule encoding a human SOX2 comprises a sequence that is at least 70% (e.g., at least 75%, 80%, 85%, 90%, 95%, 98%, 99%, or 100%) identical to a nucleic acid molecule described in the NCBI RefSeq under accession number NM_003106.4. SEQ ID NO: 3 is a non-limiting example of a nucleotide sequence encoding human SOX2. In certain embodiments, the nucleic acid molecule encoding human SOX2 comprises a sequence that is at least 70% (e.g., at least 75%, 80%, 85%, 90%, 95%, 98%, 99%, or 100%) identical to SEQ ID NO: 3. According to some embodiments, the nucleic acid molecule encodes a SOX2 comprising an amino acid sequence that is at least 70% (e.g., at least 75%, 80%, 85%, 90%, 95%, 98%, 99%, or 100%) identical to the amino acid sequence described in the NCBI RefSeq under accession number NP_003097.1. According to some embodiments, the nucleic acid molecule encodes a SOX2 comprising the amino acid sequence described in the NCBI RefSeq under accession number NP_003097.1. In some instances, the nucleic acid molecule encodes a SOX2 comprising an amino acid sequence that is at least 70% (e.g., at least 75%, 80%, 85%, 90%, 95%, 98%, 99%, or 100%) identical to SEQ ID NO: 4. SEQ ID NO: 4 is a non-limiting example of an amino acid sequence of human SOX2.

A “multicistronic vector” is a vector that encodes more than one amino acid sequence (e.g., a vector encoding OCT4 and KLF4, OCT4 and SOX2, KLF4 and SOX2, or OCT4, SOX2, and KLF4 (OSK)). A multicistronic vector allows for expression of multiple amino acid sequences from a nucleic acid sequence. Nucleic acid sequences encoding each transcription factor (e.g., OCT4, KLF4, or SOX2) may be connected or separated such that they produce unconnected proteins. For example, internal ribosome entry sites (IRES) or polypeptide cleavage signals may be placed between nucleic acid sequences encoding each transcription factor in a vector. Exemplary polypeptide cleavage signals include 2A peptides (e.g., T2A, P2A, E2A, and F2A). A T2A peptide may comprise a sequence that is at least 70% (e.g., at least 75%, 80%, 85%, 90%, 95%, 98%, 99%, or 100%) identical to SEQ ID NO: 10. A P2A peptide may comprise a sequence that is at least 70% (e.g., at least 75%, 80%, 85%, 90%, 95%, 98%, 99%, or 100%) identical to SEQ ID NO: 8. In some embodiments, an expression vector of the present disclosure is a multicistronic expression vector.

A “terminator” or “terminator sequence.” as used herein, is a nucleic acid (s, engineered nucleic acid) sequence that causes transcription to stop. A terminator may be unidirectional or bidirectional. It is comprised of a DNA sequence involved in specific termination of an RNA transcript by an RNA polymerase. A terminator sequence prevents transcriptional activation of downstream nucleic acid sequences by upstream promoters. Thus, in certain embodiments, a terminator that ends the production of an RNA transcript is contemplated.

The most commonly used type of terminator is a forward terminator. When placed downstream of a nucleic acid sequence that is usually transcribed, a forward transcriptional terminator will cause transcription to abort. In some embodiments, bidirectional transcriptional terminators may be used, which usually cause transcription to terminate on both the forward and reverse strand. In some embodiments, reverse transcriptional terminators may be used, which usually terminate transcription on the reverse strand only.

Non-limiting examples of mammalian terminator sequences include bovine growth hormone terminator, and viral termination sequences such as, for example, the SV40 terminator, spy, yejM, secG-leuU, thrLABC, rrnB Tl, hisLGDCBHAFI, metZWV, rrnC, xapR, aspA, and arcA terminator. In certain embodiments, the terminator sequence is SV40 and comprises a sequence that is at least 70% (e.g., at least 75%, 80%, 85%, 90%, 95%, 98%, 99%, or 100%) identical to SEQ ID NO: 12.

A “Tet-Off” system, as used herein, is a type of inducible system that is capable of repressing expression of a particular transgene in the presence of a tetracycline (e.g., doxycycline (DOX)). Conversely, a Tet-Off system is capable of inducing expression of a particular transgene in the absence of a tetracycline (e.g., doxycycline, DOX). In certain embodiments, a Tet-Off system comprises a tetracycline-responsive promoter operably linked to a transgene (e.g., encoding OCT4; KLF4; SOX2; or any combination thereof) and a tetracycline-controlled transactivator (tTA). The transgene with the tetracycline-responsive promoter (e.g., TRE3G, P tight, or TRE2) and the tetracycline-controlled transactivator may be encoded on the same vector or be encoded on separate vectors. See, e.g., US Publ. Appl. No. 2021-0403923 A, and the International Publ. No. WO2020/069339, entitled MUTANT REVERSE TETRACYCLINE TRANSACTIVATORS FOR EXPRESSION OF GENES, each of which is herein incorporated by reference in its entirety.

A “Tet-On” system, as used herein, is a type of inducible system that is capable of inducing expression of a particular transgene in the presence of a tetracycline (e.g., doxycycline (DOX)). In certain embodiments, a Tet-On system comprises a tetracycline-responsive promoter operably linked to a transgene (e.g., encoding OCT4; KLF4; SOX2; or any combination thereof) and a reverse tetracycline-controlled transactivator (rtTA). For example, the rtTA may be rtTA3, rtTA4, or variants thereof. In certain embodiments, a nucleic acid encoding rtTA3 comprises a sequence that is at least 70% (e.g., at least 75%, at least 80%, at least 85%, at least 90%, at least 95%, or at least 100%) identical to SEQ ID NO: 19. In certain embodiments, rtTA3 comprises an amino acid sequence that is at least 70% (e.g., at least 75%, at least 80%, at least 85%, at least 90%, at least 95%, at least 98%, at least 99%, or at least 100%) identical to SEQ ID NO: 20. In certain embodiments, a nucleic acid encoding rtTA4 comprises a sequence that is at least 70% (e.g., at least 75%, at least 80%, at least 85%, at least 90%, at least 95%, at least 98%, at least 99%, or at least 100%) identical to SEQ ID NO: 26. In certain embodiments, rtTA4 comprises an amino acid sequence that is at least 70% (e.g., at least 75%, at least 80%, at least 85%, at least 90%, at least 95%, at least 98%, at least 99%, or at least 100%) identical to SEQ ID NO: 27. The expression cassette encoding a tetracycline-responsive promoter (e.g., a promoter comprising a TRE, including TRE3G, P tight, and TRE2) and a reverse tetracycline-controlled transactivator may be encoded on the same vector or be encoded on separate vectors. See, e.g., US Publ. Appl. No. 2021-0403923 A, and the International Publ. No. WO2020/069339.

The term “tissue” refers to any biological tissue of a subject (including a group of cells, a body part, or an organ) or a part thereof, including blood and/or lymph vessels, which is the object to which a compound, particle, and/or composition as disclosed herein is delivered. A tissue may be an abnormal, damaged, or unhealthy tissue, which may need to be treated. A tissue may also be a normal or healthy tissue that is under a higher than normal risk of becoming abnormal or unhealthy, which may need to be prevented. In certain embodiments, the tissue is considered healthy but suboptimal for performance or survival in current or future conditions. In certain embodiments, the tissue is liver tissue. In certain embodiments, the tissue is damaged (e.g., due to a congenital defect, an injury, an accident, or an iatrogenic injury), diseased, and/or aged.

As used herein, a “TRE promoter” is a promoter comprising a tetracycline-responsive element (TRE). As used herein, a TRE comprises at least one (e.g., at least 2, 3, 4, 5, 6, 7, 8, 9, 10, 11, 12, 13, 14, 15, 16, 17, 18, 19, or 20) Tet-O sequences. In some embodiments, a TRE promoter further comprises a minimal promoter located downstream of a tet-O sequence. A minimal promoter is a promoter that comprises the minimal elements of a promoter (e.g., TATA box and transcription initiation site), but is inactive in the absence of an upstream enhancer (e.g., sequences comprising Tet-O). As an example, a minimal promoter may be a minimal CMV promoter that comprises a sequence that is at least 70% (e.g., at least 75%, 80%, 85%, 90%, 95%, 98%, 99%, or 100%) identical to SEQ ID NO: 17 or 24. For example, a TRE promoter may be a TRE3G promoter (e.g., a TRE3G promoter that comprises a sequence that is at least 70% (e.g., at least 75%, 80%, 85%, 90%, 95%, 98%, 99%, or 100%) identical to SEQ ID NO: 7.

The term “tissue repair” in the context of damaged tissue refers to restoration of tissue architecture, function following tissue damage, or a combination thereof. Tissue repair includes tissue regeneration, cell growth, tissue replacement, and/or rewiring of existing tissue (reprogramming).

The term “tissue regeneration” refers to production of new tissue or cells within a tissue that are the same type as the tissue of interest (e.g., same type as the damaged tissue or cell). In some embodiments, the methods provided herein promote organ regeneration.

The term “tissue replacement” refers to production of a different type of tissue compared to the tissue of interest (e.g., connective tissue to replace damaged tissue).

The term “variant” refers to a sequence that comprises a modification relative to a wild-type sequence. Non-limiting modifications in an amino acid sequence include insertions, deletions, and point mutations. Non-limiting modifications to nucleic acid sequences include frameshift mutations, nucleotide insertions, and nucleotide deletions.

The term “WPRE” refers to a Woodchuck Hepatitis Virus (WHP) Posttranscriptional Regulatory Element (WPRE). WPREs create tertiary structures in nucleic acids (e.g., expression vectors) and are capable of enhancing transgene expression (e.g., from a viral vector). In certain embodiments, a WPRE sequence is at least 70% (e.g., at least 75%, at least 80%, at least 85%, at least 90%, at least 95%, at least 98%, at least 99%, or at least 100%) identical to SEQ ID NO: 23 or 31.

### NUMBERED EMBODIMENTS

Some aspects relate to any of the following numbered embodiments:

1. A method of treating a subject having a liver disease and in need of treatment thereof or at risk of liver disease and in need of treatment thereof, comprising administering to the subject an amount of a pharmaceutical composition comprising
one or more polynucleotides encoding:
   octamer-binding transcription factor 4 (OCT4),
   sex determining region Y-box 2 (SOX2), and
   Krüppel-like factor 4 (KLF4);
wherein the one or more polynucleotides do not encode MYC proto-oncogene (c-Myc).

2. The method of embodiment 1, wherein the liver disease is a chronic liver disease.

3. The method of embodiment 1, wherein the liver disease is liver steatosis.

4. The method of embodiment 1, wherein the liver disease is liver fibrosis.

5. The method of embodiment 1, wherein the liver disease is cirrhosis.

6. The method of embodiment 1, wherein the subject has or is suspected of having metabolic dysfunction-associated steatotic liver disease.

7. The method of embodiment 1, wherein the subject has or is suspected of having metabolic dysfunction-associated steatohepatitis.

8. The method of any one of the preceding embodiments wherein a liver biopsy from the subject after the administration exhibits an improvement in one or more epigenetic markers, as compared to a baseline liver biopsy from the subject.

9. The method of embodiment 8, wherein the liver biopsy is of one or more hepatocyte cells.

10. The method of embodiment 8 or 9, wherein the liver biopsy is of one or more stellate cells.

11. The method of any one of the preceding embodiments, wherein the administration results in a biochemical response in the subject.

12. The method of any one of the preceding embodiments, wherein the administration results in an improvement in one or more biomarkers of the liver disease in the subject.

13. The method of any one of the preceding embodiments, wherein the administration results in an improvement in one or more symptoms of the subject's liver disease, as compared to baseline.

14. The method of any one of the preceding embodiments, wherein the administration results in an improvement in the stage of the subject's liver disease.

15. The method of any one of the preceding embodiments, wherein the pharmaceutical composition is administered intravenously.

16. The method of any one of the preceding embodiments, wherein the pharmaceutical composition is administered subcutaneously or intramuscularly.

17. The method of any one of the preceding embodiments, wherein the administration of the pharmaceutical composition does not induce c-Myc expression in the subject.

18. The method of any one of the preceding embodiments, wherein the pharmaceutical composition comprises a plasmid that comprises the one or more polynucleotides.

19. The method of any one of the preceding embodiments, wherein the pharmaceutical composition comprises an expression vector that comprises the one or more polynucleotides.

20. The method of embodiment 19, wherein the expression vector is a viral expression vector, for example, a lentivirus, a retrovirus, an adenovirus, alphavirus, vaccinia virus, or an adeno-associated virus (AAV) vector.

21. The method of embodiment 19 or 20, wherein the expression vector is polycistronic for OCT4, SOX2, and KLF4.

22. The method of any one of the preceding embodiments, wherein the one or more polynucleotides are in the form of deoxyribonucleic acid.

23. The method of any one of the preceding embodiments, wherein the one or more polynucleotides are in the form of ribonucleic acid (RNA).

24 The method of 23, wherein the RNA comprises mRNA.

25. The method of any one of the preceding embodiments, wherein the pharmaceutical composition comprises one or more lipid nanoparticles that comprise the one or more polynucleotides.

26. The method of any one of the preceding embodiments, wherein the OCT4, SOX2, and/or KLF4 are each operably linked to one or more promoters.

27 The method of embodiment 26, wherein at least one of the promoters is an inducible promoter.

28 The method of embodiments 26 or 27, wherein at least one of the promoters is a constitutively active promoter.

29. The method of any one of the previous embodiments, wherein at least one of the polynucleotides comprises an OCT4 sequence at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 1.

30. The method of any one of the previous embodiments, wherein at least one of the polynucleotides comprises an OCT4 sequence at least 95% identical, at least 96% identical, at least 97% identical, at least 98% identical, at least 99% identical, or 100% identical, to SEQ ID NO: 1.

31. The method of any one of the previous embodiments, wherein at least one of the polynucleotides comprises an SOX2 sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 3.

32. The method of any one of the previous embodiments, wherein at least one of the polynucleotides comprises an SOX2 sequence having at least 95% identical, at least 96% identical, at least 97% identical, at least 98% identical, at least 99% identical, or 100% identical, to SEQ ID NO: 3.

33. The method of any one of the previous embodiments, wherein at least one of the polynucleotides comprises an KLF4 sequence having at least 75% identical, at least 80% identical, at least 85% identical, at least 90 identical, at least 91% identical, at least 92% identical, at least 93% identical, at least 94% identical, at least 95% identical, at least 96% identical, at least 97% identical, at least 98% identical, at least 99% identical, or 100% identical, to SEQ ID NO: 5.

34. The method of any one of the previous embodiments, wherein at least one of the polynucleotides comprises an KLF4 sequence having at least 95% identical, at least 96% identical, at least 97% identical, at least 98% identical, at least 99% identical, or 100% identical, to SEQ ID NO: 5.

### EXAMPLES

The following examples are included for illustrative purposes only and are not intended to limit the scope of the invention.

### Example 1: Vector Production

The present study set out to develop a method for manufacturing batches of vectors and to assess the stability of the prepared batches.

### Materials

Cells: HEK293T cells were used to produce the vectors as described herein. The cells were grown in DMEM media (Invitrogen, cat. no. 11995073) containing 10% fetal bovine serum (FBS) (Invitrogen HI FBS, cat. no. 16140), penicillin/streptomycin (Invitrogen, cat. no. 15140-122), Glutamine (Invitrogen cat. no. 25030).

Plasmids: The recombinant AAV2-TRE-OSK plasmid (shown in FIG. 1) was prepared using the following components: (1) a nucleic acid sequence encoding an AAV2 capsid protein or a fragment thereof, (2) a nucleic encoding a functional rep gene, (3) a recombinant AAV transfer vector comprising AAV2 inverted terminal repeats (SEQ ID NO: 16, SEQ ID NO: 32) flanking a transgene encoding OCT4, KLF4, and SOX2 (SEQ ID NO: 13) operably linked to an inducible TRE promoter (TRE3G, SEQ ID NO: 7), and (4) a helper vector with rAAV2 Rep-Cap proteins. Plasmids were obtained from Stratagene/Agilent (Stratagene cat no: 240071). The AAV2 Rep-Cap plasmid included an pAAV-RC plasmid (Stratagene cat. no. 240071). In some cases, AAV2 hybrid vectors e.g., AAV2/1 AAV2/2, AAV2/5, AAV2/6, AAV2/7, AAV2/8 and AAV2/9 with capsid proteins from AAV1, 2, 3, 5, 6, 7, 8, and 9 serotypes. The helper plasmid included a pHelper plasmid (Stratagene, cat. no. 240071) and carried adenovirus-derived genes for introducing helper functions. As shown in FIG. 1 the entire AAV2-TRE3G-OSK-SV40 pA vector is 7250 base pairs in length, and two inverted terminal repeats (ITRs) flank the OSK sequences.

The first expression vector encoding OCT4, SOX2, and KLF4 includes the nucleic acid sequence set forth in SEQ ID NO: 15. The recombinant AAV vector can include a nucleic acid encoding an inducing agent.

The recombinant pAAV2-CMV-rtTA, pAAV2-CMV-ItTA3 (V16) (SEQ ID NO: 21), for the Tet-On plasmid was prepared using a similar approach to that described above but using a transfer plasmid with a CMV constitutive promoter (SEQ ID NO: 17) operably linked to rtTA3 (SEQ ID NO 19) inducing agent having 3 vp16 domains at the 3′ end. An alternative recombinant pAAV2-CMV-rtTA, pAAV2-CMV-ItTA4 (V16) (SEQ ID NO: 28), may be prepared in the same way.

Polyethyleneimine (PEI) Solution: PEI solution (1 μg/μl, Polysciences, cat. no. 23966-2) was prepared by dissolving PEI powder in H2O that was heated to 80° C., cooled to room temperature, neutralized to pH 7.0, filter sterilized, aliquoted and stored at −20° C. The transfection efficiency was tested when each new batch was prepared.

### Vector Production

On day 1, HEK293T cells were plated on 10 15-cm dishes before transfecting for 24 hours. Cells were seeded with 25 mL medium per 15-cm culture dish. Cells were split to a density of 70-90% (standard transfection density) ten times on 15 cm plates in order to provide a yield of 5E12 viral genomes (vg). Media was changed with 5% FBS to slow growth and reduce purification time one plate at a time in order to prevent cells from dying.

On day 2, 1 hour before transfection, medium was changed to 20 mL of freshly warmed medium. A DNA solution was prepared in a 50 mL FALCON™ tube. The amounts of DNA and reagents per dish are shown in the Table 2. Based on the size of inverted terminal repeat (ITR) plasmid, the amount of DNA was calculated. A tube of master mixture was prepared for 5 15-cm dishes. As shown in Table 2, all plasmids were diluted to 1 μg/μl in sterile H2O.

A 10-mL sample of DMEM (without phenol red, Invitrogen cat. no. 31053-036) was prepared. A 785 μl aliquot of PEI solution was added, and the media was mixed. It was then incubated at room temperature for 20-30 minutes.

TABLE 2
Amounts of reagent per plate

| | | Amount of | Amount of | | |
|---|---|---|---|---|---|
| | Plasmid | Plasmid Per | Plasmid Per | [plasmid] | |
| Plasmid | size | Plate | 5 Plates | (μg/μl) | VOLUME |
| AAV2-RC | 7.3 kb | 15.2 μg | 76 μg | 1 μg/μl | 76 μl |
| HELPER | 11.6 kb | 24 μg | 120 μg | 1 μg/μl | 120 μl |
| ITR plasmid-Gene of | 6.3 kb | 13.117808 μg | 65.58904 μg | 1 μg/μl | 65.5 μl |
| Interest (e.g., OSK) | | | | | |

Next, 2 mL of DNA-PEI mixture was added to each of 10 15-cm dishes. The transfected cells were then incubated.

On Day 3, the medium was removed, and 25 mL of freshly warmed medium was added. Serum-containing medium was added for AAV2/2, AAV2/6. Serum-free medium was used for AAV2/1, AAV2/5, AAV2/7, AAV2/8 and AAV2/9.

On Day 5, cells were scraped with a cell scraper in their current medium and transferred to a 50 mL tube. The cell suspension was then spun at 1000 relative centrifugal force (rcf) for 5 minutes. The supernatant was then discarded.

### Transfection Procedures

All cells were combined into 1 50-mL tube, washed with PBS, and spun at 1000 rpm for 5 minutes. The supernatant was then discarded.

The cell pellet was resuspended in a volume of hypotonic buffer five times the volume of the packed cell volume (approximately 25 mL). It was then incubated on ice for 10 minutes. A 0.11% by volume amount of 10× restore buffer was then added and mixed by pipetting (generally a volume of 3.3 mL).

The nuclei were then spun down at 2000 ref for 10 minutes, generating a nuclear pellet of about 1 mL per 10 15-cm dishes. The pellet was stored at −80° C. for further purification.

### Preparation of Solutions

5 M NaCl salt solution: A mass of 292.2 g NaCl was added to 200 of deionized (DI) water in a 2 L bottle. The mixture was shaken to mix, and poured into a large, graduated cylinder. DI water was then added up to 1 L and poured back into the 2 L bottle. A stir bar was added, and the volume was heated and stirred until dissolved. Alternatively, the volume was autoclaved. The solution was then cooled and sterile filtered through 0.2 μm filter.

40% PEG-8000, 2.5M NaCl (5× stock solution): In a 1 L graduated cylinder, 400 g PEG-8000 and 500 mL 5 M NaCl were mixed. DI water was added to 1 L. This generally required about 100 ml of water. The solution was transferred to a 2 L flask and shaken to mix. A large stir bar was added, and the solution was heated overnight in a water bath at 55° C. The next day, the solution was sterile filtered with a 0.2 μm filter. This filtration step generally takes about 30 minutes.

Harvesting AAV from Media and Cells

The media and cells were harvested without use of trypsin. The media was collected with a pipette and sterile filtered with a 0.2 μM filter. The cells were collected and spun down. Any remaining supernatant was added to the media sterile filtered with a 0.2 μm filter. The cells were harvested in one of several ways. Cells were collected with a cell scraper. Alternatively, cells were “blasted” with calcium- and magnesium-free PBS at a volume of 10 mL per 2 plates. A 40% solution of PEG 8000 adjusted to pH 7.4 was added to a final concentration of 12%. Approximately 25 mL per 100 mL of media/cells were used. The solution was stirred in a cold room for 1 hour and was either left to sit for 3 hours without spinning or was left overnight in cold room. The next day, the PEG mixture was spun at 3000×g for 20 minutes. The supernatant was discarded. The pellet was resuspended in less than about 7 mL of 1×PBS. The pellet was first suspended with about 5 mL and a volume of up to 2 mL was added once nearly suspended. A solution of benzonase at 1:10,000 was then added and the mixture was incubated for 45 minutes at 37° C. Optionally, the mixture was spun down at 2415×g for 10 minutes at 4° C. The supernatant was transferred to new tube.

Concentrating AAV with Ultracentrifugation

A 1 M MgCl2 stock solution was prepared which was at 1000× and was used for making MK buffer. A 2.5 M stock solution of KCl was prepared which was a 1000× and was also used for preparing MK buffer.

A 1 M solution of NaCl/phosphate-buffered saline (PBS) MK buffer was prepared by dissolving 58.4 g of NaCl in 1 mL of 1 M MgCl2 and 1 mL of 2.5 M KCl. Then, 1×PBS (Ca—Mg-Dulbecco's phosphate-buffered saline (DPBS), Gibco) was added to final volume of 1 L. The solution was sterilized by passing through a 0.22-μm filter and stored at 4° C. The solution was brought to final concentration of 1 M NaCl, 1 mM MgCl2, and 2.5 mM KCl.

A 1×PBS-MK buffer was prepared by dissolving 1 mL of 1 M MgCl2, and 1 mL 2.5 M KCl to 2×500 ml bottles of Ca—Mg-DPBS (Gibco). The solution was sterilized by passing through a 0.22-μm filter and stored at 4° C. The solution was brought to final concentrations of 1 mM MgCl2 and 2.5 mM KCl.

A 0.001% Pluronic-F68 (formulation buffer) solution was prepared by adding 500 μL of sterile 1000× Pluronic F-68 (1% solution) to 500 mL of 1×DPBS (Gibco, TC stock). The solution was stored at 4° C. for up to one month, or aliquoted and stored at −80° C. for up to one year.

A 0.001% PLURONIC™-F68+5% sorbitol (freezing buffer) solution was prepared by adding 25 g of sorbitol to 500 μL of sterile 1000×PLURONIC™ F-68 (1% solution) to 500 mL of 1×DPBS (Gibco, TC stock). The solution was stored at 4° C. for up to one month, or aliquoted and stored at −80° C. for up to one year.

As shown in Table 3, 15% iodixanol solution was prepared by mixing 30 mL of 60% iodixanol and 90 mL of 1 M NaCl/PBS-MK buffer. A 25% iodixanol solution was prepared by mixing 112.5 mL of 60% iodixanol, 157.5 mL of 1×PBS-MK buffer, and 900 μL of phenol red. A 40% iodixanol solution was prepared by mixing 202.5 mL of 60% iodixanol and 67.5 mL of 1×PBS-MK buffer. A 60% iodixanol solution was prepared by mixing 150 mL of 60% iodixanol and 675 μL of phenol red.

TABLE 3
Iodixanol solutions

| | mL 60% | mL 1M NaCl | mL PBS- | μL phenol |
|---|---|---|---|---|
| % solution | iodixanol | PBS-MK | MK | red |
| 15 | 30 | 90 | 0 | 0 |
| 25 | 112.5 | 0 | 157.5 | 675 |
| 40 | 180 | 0 | 90 | 0 |
| 60 | 150 | 0 | 0 | 675 |

*Note all solutions were sterile-filtered with 0.2 um filters.

In order to create an ultra-gradient, the benzonased supernatants were added to Beckman optiseal tubes. If volumes were unequal, 1 M NaCl, PBS-MK mix was added to make them equal, to a final volume of approximately 7 mL.

The tubes were filled from the bottom using 10 mL syringes and long hypodermic needles. For each solution the same syringe was reused but the needles were changed after every sample in order to prevent cross-contamination between AAV preparations. 5 mL was the minimum volume for any layer.

The tubes were balanced to 5-10 μg. The tubes were balanced in pairs. They were first sorted so that the most similar tubes were paired, and then PBS was added to the lighter tube in each pairing. PBS was added by touching the tip to the side of the tube to prevent droplets from disturbing the layers. Caps were added and tubes were loaded into the ultracentrifuge.

The tubes were then spun in an ultracentrifuge using a VTi50 rotor for 1 hour at max speed (50,000 rpm=242,000×g). The fractions were collected from the ultracentrifuged tubes by piercing the bottom with an 18-gauge needle. The black stopper was removed at the top before piercing with the needle, otherwise air bubbles were created that disturb the layers. The majority of the 60% fraction was removed. The remaining part of the 60% layer was then collected along with the 40% fraction in a 50 mL tube. Sample collection was complete when the color changed significantly, or the solution was cloudy Separation of fractions on a protein gel

Samples were denatured with 4×LDS with 2.5% β-mercaptoethanol at 70° C. for 10 minutes. In order to prepare 20 μL aliquots, 10 μL of sample was combined with 5 μL of H2O, 5 μL of LDS with 2.5% β-mercaptoethanol. If necessary, extra running buffer (200 mL 10× Tris-Glycine SDS buffer, 1800 mL Millipure water) was prepared.

Samples were then loaded into a Tris Glycine gel. Either a 4-12% or 4-20% gradient gel was used. For AAV capsids, either is suitable.

The gel box was assembled, and the wells of the gel were loaded with 20 μL of sample per well. Gels were run at 225 V for 30-45 minutes, until the blue dye reached the bottom of the gel.

The gels were then stained with SYPRO™ red and imaged. First, a staining solution was prepared (7.5% acetic acid, SYPRO™ red is 5000×) by adding 10 μL of SYPRO™ red to 50 mL of stain solution. This was enough for 1 gel. Gels were stained for 1 hour while covered at room temperature on a slow rocker. The stain solution was then removed and acetic acid solution with no dye was added. The gels were incubated for 1 to 5 minutes in order to de-stain. The gels were images on a gel dock with EtBr settings (UV).

Good fractions were then combined and washed with 1×PBS with 0.001% F68. In some cases about 20 mL PBS+F68 was added to bring the final volume to just less than 30 mL in order to dilute the iodixanol which facilitates passing through the filter.

Samples were spun at 4700 g for 5 minutes. After everything flowed through, the samples were washed 3× more with 15 mL per wash in order to prepare a clear final solution. The samples were then aliquoted into labelled tubes (name of virus, payload, date) and stored at 4° C. for up to 1 week. For longer storage, the final elution was prepared with 1×PBS with 0.001% F68 and 5% sorbitol. Samples were frozen at −80° C.

Titering Virus with qPCR

Samples were prepared by aliquoting 12.5 μl Master Mix into tubes. Either fast advanced TAQMAN™ Master Mix from ThermoFisher or IDT Primetime Master Mix was used. To each tube, 0.0625 μl of primer 1, 0.0625 μl of primer 2, 0.125 μl of probe, 1 μl of virus, and 11.3 μl of H2O was added for a total volume of 25 μl.

### Example 2: LNP Administration

Lipid nanoparticles (LNPs) in Table 4 were generated. Each of the LNPs included an indicated in the table, as well as cholesterol, a helper lipid (LNP #1, LNP #2, and LNP #4: DOPE; LNP #3: DOPC), and a PEGylated lipid (DMG-PEG-2K in this case). The LNPs further included an mRNA encoding OCT4, SOX2, and KLF4 (OSK).

TABLE 4
LNPs

| | | | | | Zeta | | | |
|---|---|---|---|---|---|---|---|---|
| | Ionizable | Formulation | Size | | Potential | EE | Conc | Yield |
| LNP # | Lipid | Buffer | (nm) | PDI | (mV) | (%) | (μg/mL) | (%) |
| LNP | MC3 | Citrate, pH 3 | 81 | 0.12 | −0.1 | 96 | 243 | 51 |
| #1 | | | | | | | | |
| LNP | ALC- | Citrate, pH 5 | 110 | 0.19 | −8.6 | 93 | 259 | 68 |
| #2 | 0315 | | | | | | | |
| LNP | SS-OP | Citrate, pH 5 | 94 | 0.21 | 2.0 | 91 | 243 | 62 |
| #3 | N/P 16 | | | | | | | |
| LNP | LP-01 | Citrate, pH 3 | 87 | 0.09 | 0.5 | 96 | 251 | 64 |
| #4 | | | | | | | | |

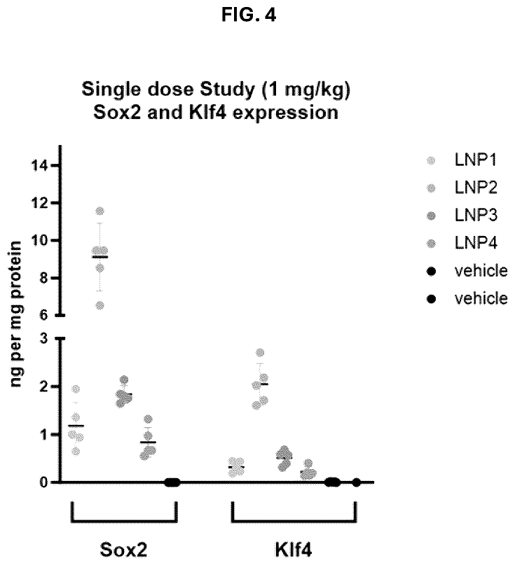

The LNPs were administered in a single 1 mg/kg intravenous dose to mice (n=5/group). OSK protein expression was determined in homogenized perfused liver tissue by an enzyme linked immunosorbent assay (ELISA). The ELISA included 3 replicates per sample. Resulting protein expression data are shown in FIG. 4, which shows increases in SOX2 and KLF4 expression in livers in response to administration of LNP #1, LNP #2, LNP #3, and LNP #4, relative to a negative control which included vehicle only. Similar results are expected for OCT4 expression in response to the LNPs. LNP #2 administration resulted in greater liver OSK expression than the other LNPs in this experiment. No expression seen in response to the negative control.

### Example 3: Liver Disease Treatment

Studies will be performed to determine whether epigenetic reprogramming can improve liver disease symptoms of rodent mouse models. In one set of experiments, a Gubra amylin NASH (GAN) diet-induced MASH mouse model will be used, for example as described in Vacca, M. et al. An unbiased ranking of murine dietary models based on their proximity to human metabolic dysfunction-associated steatotic liver disease (MASLD). Nat Metab 6, 1178-1196 (2024), which is incorporated herein by reference in its entirety. Mice will be provided a diet that includes 40% fat of mostly palm oil, 40% carbohydrates of which half is fructose, 2% cholesterol, and 18% protein.

Mice will be provided a treatment of nucleic acids encoding partial reprogramming factors OCT4, SOX2, KLF4 delivered as DNA using an AAV vector or delivered as mRNA in lipid nanoparticles (LNPs), or will receive a negative control treatment such as vehicle empty vector. Treatment groups will be provided at various time points, including an early treatment group prior to liver disease symptom generation, a group that receives treatment upon identification of liver disease symptoms (e.g. 1 week after liver disease symptoms such as liver steatosis occurs), and later-stage such as after liver fibrosis occurs. In addition to morphological and histological liver measurements, epigenetic changes will be determined as well, including methylation assessment via DNA sequencing and fibrosis-related gene expression assessed by RNA sequencing, with the hypothesis that treatment may improve any of these outcomes.

Tests will be performed with and without a liver targeting moiety. Some treatment groups will include an LNP without a targeting ligand for passive targeting for hepatocytes. In addition, a retinoid conjugated LNP will be tested for active targeting for HSCs.

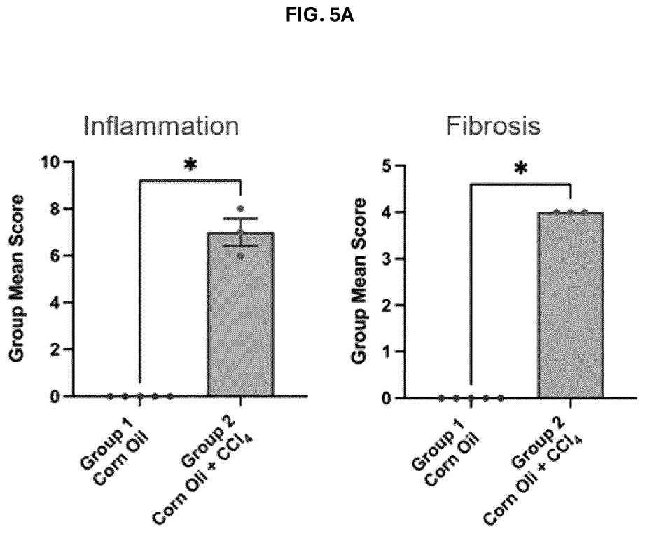

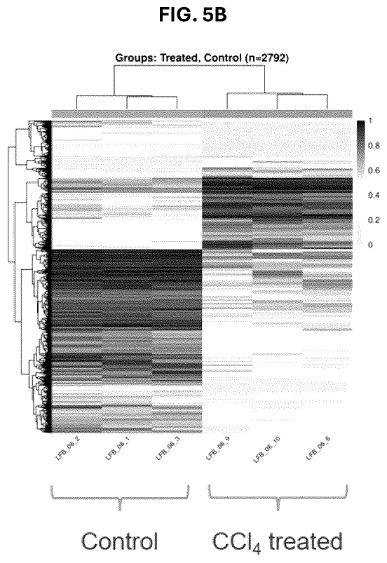

Additional follow-up studies will be performed using another model system, which includes CCl₄, and which will include a similar hypothesis that treatment with nucleic acids encoding partial reprogramming factors may improve at least some physiological indications or biomarkers associated with liver steatosis or fibrosis. As shown in FIG. 5A-5B, CCl₄ is used to induce liver fibrosis and inflammation, and cause DNA methylation changes in mice, where the fibrosis and inflammation are associated with differential DNA methylation changes. OSK treatment as provided herein may reduce the liver inflammation, liver fibrosis, or inhibit or reverse methylation changes induced by CCl₄. This may demonstrate rejuvenation of liver cells, reversion to an earlier stage of disease, and improvement in one or more symptoms of the MASLD or MASH.

### Example 4: Clinical Trial

Studies will be performed in human subjects who have metabolic dysfunction-associated steatotic liver disease (MASLD) and metabolic dysfunction-associated steatohepatitis (MASH). Nucleic acids encoding partial reprogramming factors OCT4, SOX2, KLF4 will be delivered either as DNA using an AAV vector or via mRNA in a lipid nanoparticle. Negative control treatments groups will receive empty vector or empty nanoparticles. Subjects treated with nucleic acids encoding partial reprogramming factors via either delivery method are expected to demonstrate rejuvenated liver cells, reversion to an earlier stage of disease, and possible improvement in one or more symptoms of the MASLD or MASH relative to a baseline measurement or relative to a negative control treatment group.

### Example 5: Treatment of MASH

The present Example demonstrates the treatment of MASH by OSK therapy in a mouse model of disease. Results demonstrate remarkable improvement in disease phenotypes, without loss of body weight, including significant improvement in each of body weight, liver weight, total cholesterol in plasma, alanine transaminase (ALT) in plasma, aspartate transaminase (AST) in plasma, total bile acids in plasma, NAFLD (nonalcoholic fatty liver disease) score, steatosis score, and percentage of hepatocytes with lipid droplets. The present Example utilized the DIO mouse model, also referred to as the Gubra GAN DIO-MASH mouse model or GAN DIO-MASH model. In this model, male C57BL/6J mice were fed a Gubra Amylin NASH (GAN) diet for a minimum of 30 weeks, which diet included 40% fat (mostly palm oil), 40% carbohydrates (20% fructose), and 2% cholesterol. This model is known to mirror the metabolic burden of human MASH and provide a clinically relevant model of MASH.

In the present Example, mice were fed the GAN diet for 36 to 38 weeks before being randomly stratified into experimental and control groups, with measurement of baseline metrics of disease four weeks prior to intravenous administration of a single dose of AAV vector delivering a nucleic acid payload encoding OSK (AAV-OSK), or a vehicle control. After administration of the OSK therapy or vehicle control, mice were maintained on the GAN diet for a study period of 2-3 months.

Data collected during the present Example included measurements of body weight, liver weight, total cholesterol, alanine transaminase (ALT), aspartate transaminase (AST), total bile acids, NAFLD (nonalcoholic fatty liver disease) score, steatosis score, and percentage of hepatocytes with lipid droplets. The effect of treatment on the NAFLD score was measured as the percentage of animals showing greater than one point improvement in the NAFLD score. The effect of treatment on the steatosis score was measured as the percentage of animals showing greater than one point improvement in the steatosis score. NAFLD score was determined from Hematoxylin and Eosin-stained liver samples based as a sum of scores for steatosis, inflammation and ballooning (see, e.g., Kleiner 2005 Hepatology 41 (6); 1313-21). Steatosis score was calculated based on percentage of hepatocytes with lipid droplets (<5%-score 0, 5-33%-score 1, >33-66%-score 2, >66% score 3).

Data demonstrated remarkable efficacy for treatment of MASH, including improvement across numerous metrics of disease. Moreover, the measured improvements in the metrics of disease were achieved without concomitant significant loss of body weight. Mice treated with OSK therapy showed consistent improvement in liver weight, plasma total cholesterol, plasma ALT, plasma AST, plasma total bile acids, NAFLD score, steatosis score, and percentage of hepatocytes with lipid droplets.

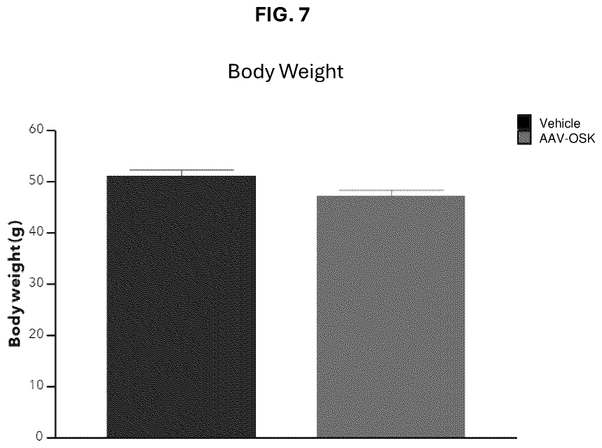

FIG. 7 compares body weight of mice administered OSK therapy and body weight of control mice. Body weight did not significantly decrease in mice administered OSK therapy as compared to control mice.

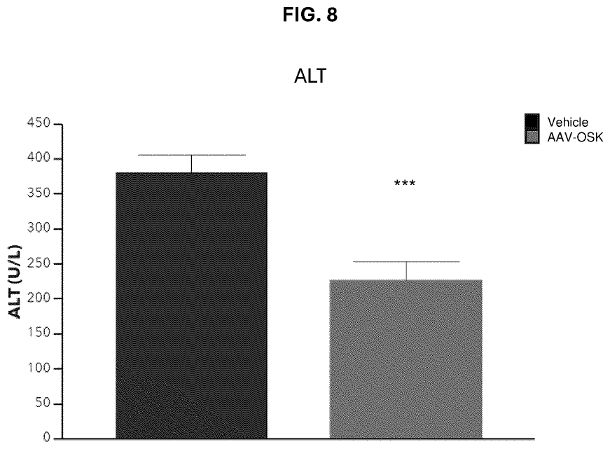

FIG. 8 compares plasma ALT in mice administered OSK therapy and plasma ALT in control mice. ALT was significantly reduced in mice administered OSK therapy as compared to control mice.

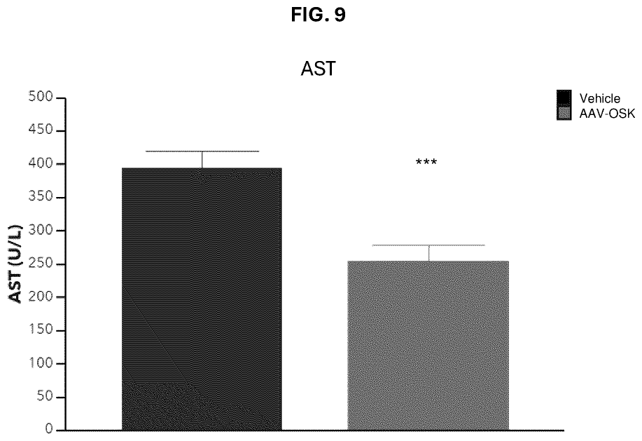

FIG. 9 compares plasma AST in mice administered OSK therapy and plasma AST in control mice. AST was significantly reduced in mice administered OSK therapy as compared to control mice.

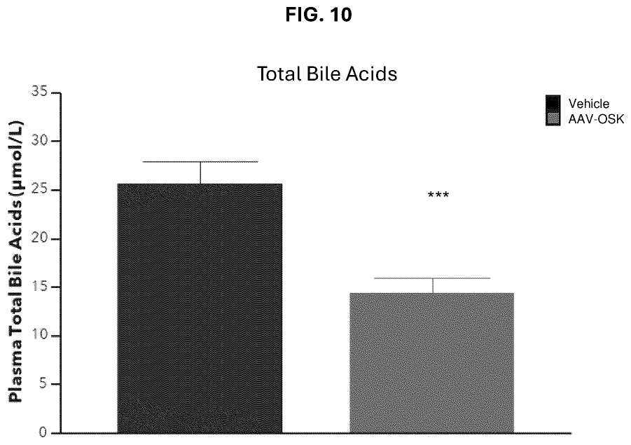

FIG. 10 compares plasma total bile acids in mice administered OSK therapy and plasma total bile acids in control mice. Total bile acids were significantly reduced in mice administered OSK therapy as compared to control mice.

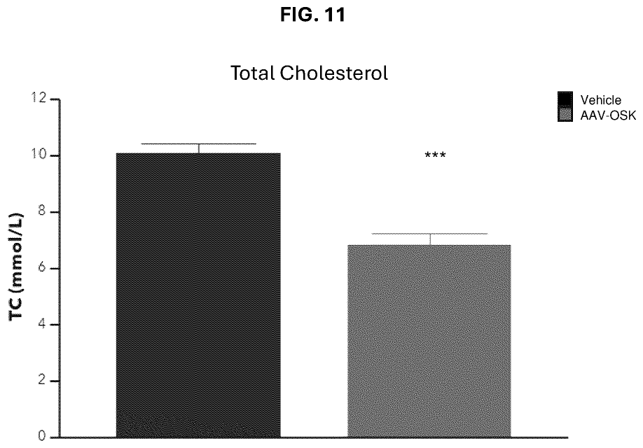

FIG. 11 compares plasma total cholesterol in mice administered OSK therapy and plasma total cholesterol in control mice. Total cholesterol was significantly reduced in mice administered OSK therapy as compared to control mice.

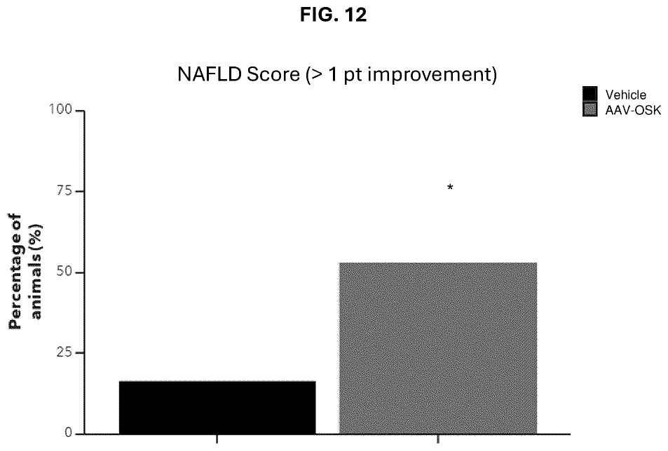

FIG. 12 compares NAFLD score in mice administered OSK therapy and NAFLD score in control mice. NAFLD score was significantly improved in mice administered OSK therapy as compared to control mice.

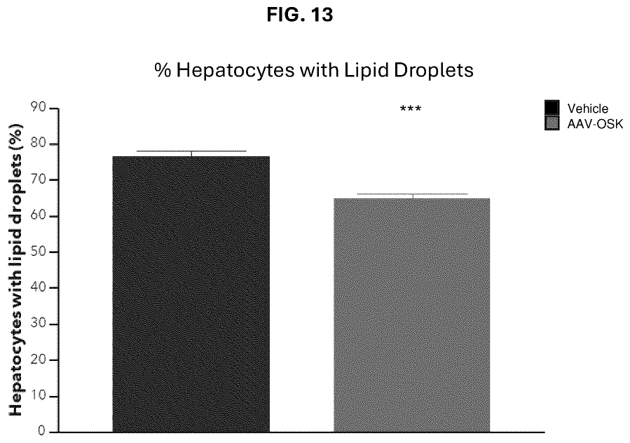

FIG. 13 compares percentage of hepatocytes with lipid droplets in mice administered OSK therapy and percentage of hepatocytes with lipid droplets in control mice. Percentage of hepatocytes with lipid droplets was significantly reduced in mice administered OSK therapy as compared to control mice.

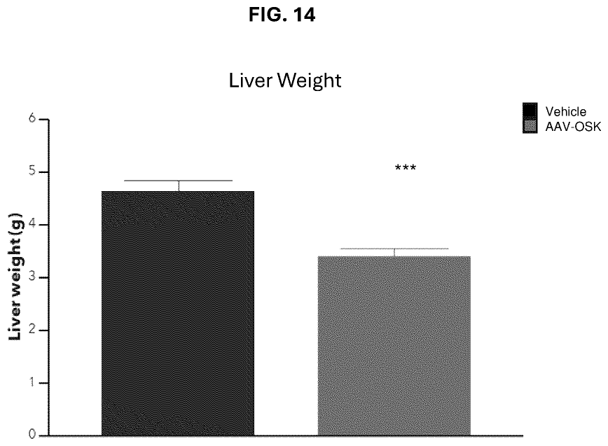

FIG. 14 compares liver weight in mice administered OSK therapy and liver weight in control mice. Liver weight was significantly lowered in mice administered OSK therapy as compared to control mice.

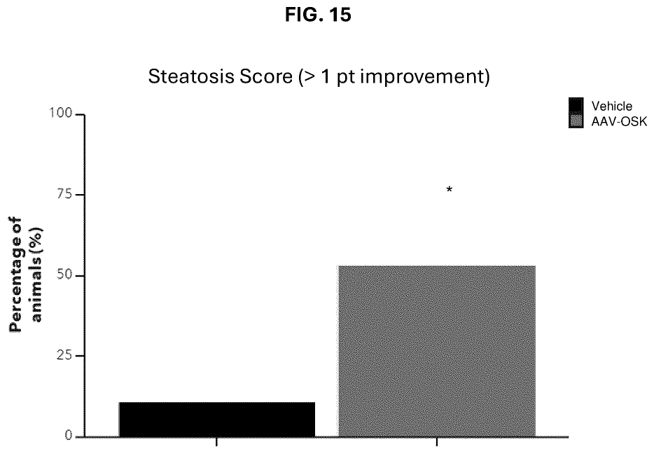

FIG. 15 compares steatosis score in mice administered OSK therapy and steatosis score in control mice. Steatosis score was significantly improved in mice administered OSK therapy as compared to control mice.

### Example 6: Detection of Protein Expression in Liver

The present Example demonstrates that OSK therapy increased expression of OSK proteins in liver, in a mouse model of MASH. The present Example utilized the DIO mouse model, also referred to as the Gubra GAN DIO-MASH mouse model or GAN DIO-MASH model. In this model, male C57BL/6J mice are fed a Gubra Amylin NASH (GAN) diet for a minimum of 30 weeks, which diet included 40% fat (mostly palm oil), 40% carbohydrates (20% fructose), and 2% cholesterol. This model is known to mirror the metabolic burden of human MASH and provide a clinically relevant model of MASH.

In the present Example, mice were fed the GAN diet for 36 to 38 weeks before being randomly stratified into experimental and control groups and respectively administered a single intravenous dose of AAV vector delivering a nucleic acid payload encoding OSK (AAV-OSK), or a vehicle control. After administration of the OSK therapy or vehicle control, mice were maintained on the GAN diet for 2-3 months, at the conclusion of which liver tissue was collected for analysis.

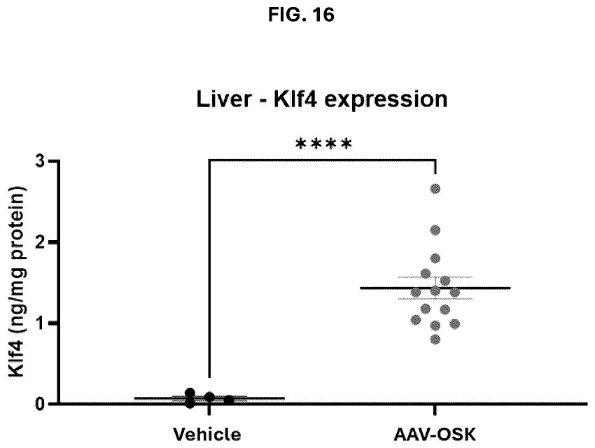

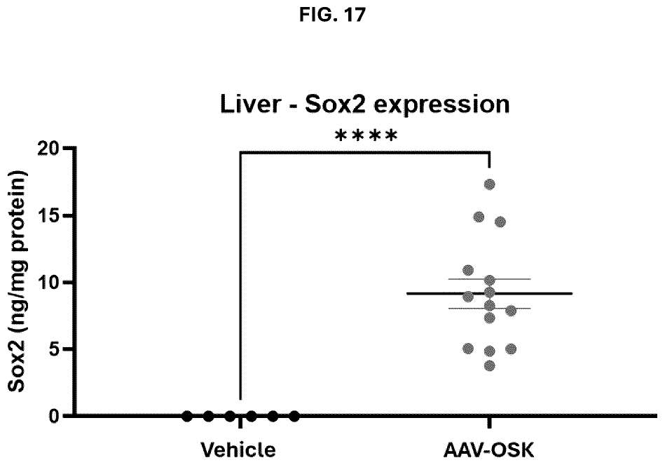

KLF4 and SOX2 protein levels were measured in homogenized perfused liver tissue by an enzyme linked immunosorbent assay (ELISA). As shown in FIGS. 16 and 17, results demonstrated increased expression of KLF4 and SOX2 protein in the liver tissue of mice administered OSK therapy as compared to mice that received the vehicle control.

TABLE 5
SEQUENCES

| SEQ ID | | |
|---|---|---|
| NO: | Component | Sequence |
| 1 | Human OCT4 | ATGGCGGGACACCTGGCTTCGGATTTCGCCTTCTCGCCCCCTCCAGGTGG |
| | nucleic acid | TGGAGGTGATGGGCCAGGGGGGCCGGAGCCGGGCTGGGTTGATCCTCGGA |
| | sequence | CCTGGCTAAGCTTCCAAGGCCCTCCTGGAGGGCCAGGAATCGGGCCGGGG |
| | | GTTGGGCCAGGCTCTGAGGTGTGGGGGATTCCCCCATGCCCCCCGCCGTA |
| | | TGAGTTCTGTGGGGGGATGGCGTACTGTGGGCCCCAGGTTGGAGTGGGGC |
| | | TAGTGCCCCAAGGCGGCTTGGAGACCTCTCAGCCTGAGGGCGAAGCAGGA |
| | | GTCGGGGTGGAGAGCAACTCCGATGGGGCCTCCCCGGAGCCCTGCACCGT |
| | | CACCCCTGGTGCCGTGAAGCTGGAGAAGGAGAAGCTGGAGCAAAACCCGG |
| | | AGGAGTCCCAGGACATCAAAGCTCTGCAGAAAGAACTCGAGCAATTTGCC |
| | | AAGCTCCTGAAGCAGAAGAGGATCACCCTGGGATATACACAGGCCGATGT |
| | | GGGGCTCACCCTGGGGGTTCTATTTGGGAAGGTATTCAGCCAAACGACCA |
| | | TCTGCCGCTTTGAGGCTCTGCAGCTTAGCTTCAAGAACATGTGTAAGCTG |
| | | CGGCCCTTGCTGCAGAAGTGGGTGGAGGAAGCTGACAACAATGAAAATCT |
| | | TCAGGAGATATGCAAAGCAGAAACCCTCGTGCAGGCCCGAAAGAGAAAGC |
| | | GAACCAGTATCGAGAACCGAGTGAGAGGCAACCTGGAGAATTTGTTCCTG |
| | | CAGTGCCCGAAACCCACACTGCAGCAGATCAGCCACATCGCCCAGCAGCT |
| | | TGGGCTCGAGAAGGATGTGGTCCGAGTGTGGTTCTGTAACCGGCGCCAGA |
| | | AGGGCAAGCGATCAAGCAGCGACTATGCACAACGAGAGGATTTTGAGGCT |
| | | GCTGGGTCTCCTTTCTCAGGGGGACCAGTGTCCTTTCCTCTGGCCCCAGG |
| | | GCCCCATTTTGGTACCCCAGGCTATGGGAGCCCTCACTTCACTGCACTGT |
| | | ACTCCTCGGTCCCTTTCCCTGAGGGGGAAGCCTTTCCCCCTGTCTCTGTC |
| | | ACCACTCTGGGCTCTCCCATGCATTCAAAC |
| 2 | Human OCT4 | MAGHLASDFAFSPPPGGGGDGPGGPEPGWVDPRTWLSFQGPPGGPGIGPG |
| | amino acid | VGPGSEVWGIPPCPPPYEFCGGMAYCGPQVGVGLVPQGGLETSQPEGEAG |
| | sequence | VGVESNSDGASPEPCTVTPGAVKLEKEKLEQNPEESQDIKALQKELEQFA |
| | | KLLKQKRITLGYTQADVGLTLGVLFGKVFSQTTICRFEALQLSFKNMCKL |
| | | RPLLQKWVEEADNNENLQEICKAETLVQARKRKRTSIENRVRGNLENLFL |
| | | QCPKPTLQQISHIAQQLGLEKDVVRVWFCNRRQKGKRSSSDYAQREDFEA |
| | | AGSPFSGGPVSFPLAPGPHFGTPGYGSPHFTALYSSVPFPEGEAFPPVSV |
| | | TTLGSPMHSN |
| 3 | Human SOX2 | ATGTACAACATGATGGAGACGGAGCTGAAGCCGCCGGGCCCGCAGCAAAC |
| | nucleic acid | TTCGGGGGGCGGCGGCGGCAACTCCACCGCGGCGGCGGCCGGCGGCAACC |
| | sequence | AGAAAAACAGCCCGGACCGCGTCAAGCGGCCCATGAATGCCTTCATGGTG |
| | | TGGTCCCGCGGGCAGCGGCGCAAGATGGCCCAGGAGAACCCCAAGATGCA |
| | | CAACTCGGAGATCAGCAAGCGCCTGGGCGCCGAGTGGAAACTTTTGTCGG |
| | | AGACGGAGAAGCGGCCGTTCATCGACGAGGCTAAGCGGCTGCGAGCGCTG |
| | | CACATGAAGGAGCACCCGGATTATAAATACCGGCCCCGGCGGAAAACCAA |
| | | GACGCTCATGAAGAAGGATAAGTACACGCTGCCCGGCGGGCTGCTGGCCC |
| | | CCGGCGGCAATAGCATGGCGAGCGGGGTCGGGGTGGGCGCCGGCCTGGGC |
| | | GCGGGCGTGAACCAGCGCATGGACAGTTACGCGCACATGAACGGCTGGAG |
| | | CAACGGCAGCTACAGCATGATGCAGGACCAGCTGGGCTACCCGCAGCACC |
| | | CGGGCCTCAATGCGCACGGCGCAGCGCAGATGCAGCCCATGCACCGCTAC |
| | | GACGTGAGCGCCCTGCAGTACAACTCCATGACCAGCTCGCAGACCTACAT |
| | | GAACGGCTCGCCCACCTACAGCATGTCCTACTCGCAGCAGGGCACCCCTG |
| | | GCATGGCTCTTGGCTCCATGGGTTCGGTGGTCAAGTCCGAGGCCAGCTCC |
| | | AGCCCCCCTGTGGTTACCTCTTCCTCCCACTCCAGGGCGCCCTGCCAGGC |
| | | CGGGGACCTCCGGGACATGATCAGCATGTATCTCCCCGGCGCCGAGGTGC |
| | | CGGAACCCGCCGCCCCCAGCAGACTTCACATGTCCCAGCACTACCAGAGC |
| | | GGCCCGGTGCCCGGCACGGCCATTAACGGCACACTGCCCCTCTCACACAT |
| | | G |
| 4 | Human SOX2 | MYNMMETELKPPGPQQTSGGGGGNSTAAAAGGNQKNSPDRVKRPMNAFMV |
| | amino acid | WSRGQRRKMAQENPKMHNSEISKRLGAEWKLLSETEKRPFIDEAKRLRAL |
| | sequence | HMKEHPDYKYRPRRKTKTLMKKDKYTLPGGLLAPGGNSMASGVGVGAGLG |
| | | AGVNQRMDSYAHMNGWSNGSYSMMQDQLGYPQHPGLNAHGAAQMQPMHRY |
| | | DVSALQYNSMTSSQTYMNGSPTYSMSYSQQGTPGMALGSMGSVVKSEASS |
| | | SPPVVTSSSHSRAPCQAGDLRDMISMYLPGAEVPEPAAPSRLHMSQHYQS |
| | | GPVPGTAINGTLPLSHM |
| 5 | Human KLF4 | ATGGCTGTCAGCGACGCGCTGCTCCCATCTTTCTCCACGTTCGCGTCTGG |
| | nucleic acid | CCCGGCGGGAAGGGAGAAGACACTGCGTCAAGCAGGTGCCCCGAATAACC |
| | sequence | GCTGGCGGGAGGAGCTCTCCCACATGAAGCGACTTCCCCCAGTGCTTCCC |
| | | GGCCGCCCCTATGACCTGGCGGCGGCGACCGTGGCCACAGACCTGGAGAG |
| | | CGGCGGAGCCGGTGCGGCTTGCGGCGGTAGCAACCTGGCGCCCCTACCTC |
| | | GGAGAGAGACCGAGGAGTTCAACGATCTCCTGGACCTGGACTTTATTCTC |
| | | TCCAATTCGCTGACCCATCCTCCGGAGTCAGTGGCCGCCACCGTGTCCTC |
| | | GTCAGCGTCAGCCTCCTCTTCGTCGTCGCCGTCGAGCAGCGGCCCTGCCA |
| | | GCGCGCCCTCCACCTGCAGCTTCACCTATCCGATCCGGGCCGGGAACGAC |
| | | CCGGGCGTGGCGCCGGGCGGCACGGGCGGAGGCCTCCTCTATGGCAGGGA |
| | | GTCCGCTCCCCCTCCGACGGCTCCCTTCAACCTGGCGGACATCAACGACG |
| | | TGAGCCCCTCGGGCGGCTTCGTGGCCGAGCTCCTGCGGCCAGAATTGGAC |
| | | CCGGTGTACATTCCGCCGCAGCAGCCGCAGCCGCCAGGTGGCGGGCTGAT |
| | | GGGCAAGTTCGTGCTGAAGGCGTCGCTGAGCGCCCCTGGCAGCGAGTACG |
| | | GCAGCCCGTCGGTCATCAGCGTCAGCAAAGGCAGCCCTGACGGCAGCCAC |
| | | CCGGTGGTGGTGGCGCCCTACAACGGCGGGCCGCCGCGCACGTGCCCCAA |
| | | GATCAAGCAGGAGGCGGTCTCTTCGTGCACCCACTTGGGCGCTGGACCCC |
| | | CTCTCAGCAATGGCCACCGGCCGGCTGCACACGACTTCCCCCTGGGGCGG |
| | | CAGCTCCCCAGCAGGACTACCCCGACCCTGGGTCTTGAGGAAGTGCTGAG |
| | | CAGCAGGGACTGTCACCCTGCCCTGCCGCTTCCTCCCGGCTTCCATCCCC |
| | | ACCCGGGGCCCAATTACCCATCCTTCCTGCCCGATCAGATGCAGCCGCAA |
| | | GTCCCGCCGCTCCATTACCAAGAGCTCATGCCACCCGGTTCCTGCATGCC |
| | | AGAGGAGCCCAAGCCAAAGAGGGGAAGACGATCGTGGCCCCGGAAAAGGA |
| | | CCGCCACCCACACTTGTGATTACGCGGGCTGCGGCAAAACCTACACAAAG |
| | | AGTTCCCATCTCAAGGCACACCTGCGAACCCACACAGGTGAGAAACCTTA |
| | | CCACTGTGACTGGGACGGCTGTGGATGGAAATTCGCCCGCTCAGATGAAC |
| | | TGACCAGGCACTACCGTAAACACACGGGGCACCGCCCGTTCCAGTGCCAA |
| | | AAATGCGACCGAGCATTTTCCAGGTCGGACCACCTCGCCTTACACATGAA |
| | | GAGGCATTTT |
| 6 | Human KLF4 | MAVSDALLPSFSTFASGPAGREKTLRQAGAPNNRWREELSHMKRLPPVLP |
| | amino acid | GRPYDLAAATVATDLESGGAGAACGGSNLAPLPRRETEEFNDLLDLDFIL |
| | sequence | SNSLTHPPESVAATVSSSASASSSSSPSSSGPASAPSTCSFTYPIRAGND |
| | | PGVAPGGTGGGLLYGRESAPPPTAPFNLADINDVSPSGGFVAELLRPELD |
| | | PVYIPPQQPQPPGGGLMGKFVLKASLSAPGSEYGSPSVISVSKGSPDGSH |
| | | PVVVAPYNGGPPRTCPKIKQEAVSSCTHLGAGPPLSNGHRPAAHDFPLGR |
| | | QLPSRTTPTLGLEEVLSSRDCHPALPLPPGFHPHPGPNYPSFLPDQMQPQ |
| | | VPPLHYQELMPPGSCMPEEPKPKRGRRSWPRKRTATHTCDYAGCGKTYTK |
| | | SSHLKAHLRTHTGEKPYHCDWDGCGWKFARSDELTRHYRKHTGHRPFQCQ |
| | | KCDRAFSRSDHLALHMKRHF |
| 7 | TRE3G (TRE | TTTACTCCCTATCAGTGATAGAGAACGTATGAAGAGTTTACTCCCTATCA |
| | promoter) | GTGATAGAGAACGTATGCAGACTTTACTCCCTATCAGTGATAGAGAACGT |
| | | ATAAGGAGTTTACTCCCTATCAGTGATAGAGAACGTATGACCAGTTTACT |
| | | CCCTATCAGTGATAGAGAACGTATCTACAGTTTACTCCCTATCAGTGATA |
| | | GAGAACGTATATCCAGTTTACTCCCTATCAGTGATAGAGAACGTATAAGC |
| | | TTTAGGCGTGTACGGTGGGCGCCTATAAAAGCAGAGCTCGTTTAGTGAAC |
| | | CGTCAGATCGCCTGGAGCAATTCCACAACACTTTTGTCTTATACCAACTT |
| | | TCCGTACCACTTCCTACCCTCGTAAA |
| 8 | P2A peptide | GCCACGAACTTCTCTCTGTTAAAGCAAGCAGGAGATGTTGAAGAAAACCC |
| | from porcine | CGGGCCT |
| | teschovirus-1 | |
| | polyprotein | |
| 9 | P2A amino acid | ATNFSLLKQAGDVEENPGP |
| 10 | T2A peptide | GAGGGCAGGGGAAGTCTTCTAACATGCGGGGACGTGGAGGAAAATCCCGG |
| | from Thosea | CCCA |
| | asigna virus | |
| | capsid protein | |
| 11 | T2A cleavage | EGRGSLLTCGDVEENPGP |
| | sequence amino | |
| | acid | |
| 12 | SV40 | AACTTGTTTATTGCAGCTTATAATGGTTACAAATAAAGCAATAGCATCAC |
| | | AAATTTCACAAATAAAGCATTTTTTTCACTGCATTCTAGTTGTGGTTTGT |
| | | CCAAACTCATCAATGTATCTTATCATGTCTGGATC |
| 13 | OCT4-2A- | CTATGCGGCATCAGAGCAGATTGTACTGAGAGTGCACCATATGCGGTGTG |
| | SOX2-2A- | AAATACCGCACAGATGCGTAAGGAGAAAATACCGCATCAGGCGCCATTCG |
| | KLF4 (Whole | CCATTCAGGCTGCGCAACTGTTGGGAAGGGCGATCGGTGCGGGCCTCTTC |
| | insert sequence | GCTATTACGCCAGCTGGCGAAAGGGGGATGTGCTGCAAGGCGATTAAGTT |
| | including Tet | GGGTAACGCCAGGGTTTTCCCAGTCACGACGTTGTAAAACGACGGCCAGT |
| | operators and | GAATTGACGCGTATTGGGATCCATGATTACGCCAGATTTAATTAAGGCCT |
| | SV40, minus | TAATTAGGCTGCGCGCTCGCTCGCTCACTGAGGCCGCCCGGGCAAAGCCC |
| | ITRs) | GGGCGTCGGGCGACCTTTGGTCGCCCGGCCTCAGTGAGCGAGCGAGCGCG |
| | | CAGAGAGGGAGTGGCCAACTCCATCACTAGGGGTTCCTTGTAGTTAATGA |
| | | TTAACCCGCCATGCTACTTATCTACGTAGCCATGCTCTAGGAAGATCGGA |
| | | ATTCTTTACTCCCTATCAGTGATAGAGAACGTATGAAGAGTTTACTCCCT |
| | | ATCAGTGATAGAGAACGTATGCAGACTTTACTCCCTATCAGTGATAGAGA |
| | | ACGTATAAGGAGTTTACTCCCTATCAGTGATAGAGAACGTATGACCAGTT |
| | | TACTCCCTATCAGTGATAGAGAACGTATCTACAGTTTACTCCCTATCAGT |
| | | GATAGAGAACGTATATCCAGTTTACTCCCTATCAGTGATAGAGAACGTAT |
| | | AAGCTTTAGGCGTGTACGGTGGGCGCCTATAAAAGCAGAGCTCGTTTAGT |
| | | GAACCGTCAGATCGCCTGGAGCAATTCCACAACACTTTTGTCTTATACCA |
| | | ACTTTCCGTACCACTTCCTACCCTCGTAAAGCGGCCGCGCCACCATGGCG |
| | | GGACACCTGGCTTCGGATTTCGCCTTCTCGCCCCCTCCAGGTGGTGGAGG |
| | | TGATGGGCCAGGGGGGCCGGAGCCGGGCTGGGTTGATCCTCGGACCTGGC |
| | | TAAGCTTCCAAGGCCCTCCTGGAGGGCCAGGAATCGGGCCGGGGGTTGGG |
| | | CCAGGCTCTGAGGTGTGGGGGATTCCCCCATGCCCCCCGCCGTATGAGTT |
| | | CTGTGGGGGGATGGCGTACTGTGGGCCCCAGGTTGGAGTGGGGCTAGTGC |
| | | CCCAAGGCGGCTTGGAGACCTCTCAGCCTGAGGGCGAAGCAGGAGTCGGG |
| | | GTGGAGAGCAACTCCGATGGGGCCTCCCCGGAGCCCTGCACCGTCACCCC |
| | | TGGTGCCGTGAAGCTGGAGAAGGAGAAGCTGGAGCAAAACCCGGAGGAGT |
| | | CCCAGGACATCAAAGCTCTGCAGAAAGAACTCGAGCAATTTGCCAAGCTC |
| | | CTGAAGCAGAAGAGGATCACCCTGGGATATACACAGGCCGATGTGGGGCT |
| | | CACCCTGGGGGTTCTATTTGGGAAGGTATTCAGCCAAACGACCATCTGCC |
| | | GCTTTGAGGCTCTGCAGCTTAGCTTCAAGAACATGTGTAAGCTGCGGCCC |
| | | TTGCTGCAGAAGTGGGTGGAGGAAGCTGACAACAATGAAAATCTTCAGGA |
| | | GATATGCAAAGCAGAAACCCTCGTGCAGGCCCGAAAGAGAAAGCGAACCA |
| | | GTATCGAGAACCGAGTGAGAGGCAACCTGGAGAATTTGTTCCTGCAGTGC |
| | | CCGAAACCCACACTGCAGCAGATCAGCCACATCGCCCAGCAGCTTGGGCT |
| | | CGAGAAGGATGTGGTCCGAGTGTGGTTCTGTAACCGGCGCCAGAAGGGCA |
| | | AGCGATCAAGCAGCGACTATGCACAACGAGAGGATTTTGAGGCTGCTGGG |
| | | TCTCCTTTCTCAGGGGGACCAGTGTCCTTTCCTCTGGCCCCAGGGCCCCA |
| | | TTTTGGTACCCCAGGCTATGGGAGCCCTCACTTCACTGCACTGTACTCCT |
| | | CGGTCCCTTTCCCTGAGGGGGAAGCCTTTCCCCCTGTCTCTGTCACCACT |
| | | CTGGGCTCTCCCATGCATTCAAACGCTAGCGGCAGCGGCGCCACGAACTT |
| | | CTCTCTGTTAAAGCAAGCAGGAGATGTTGAAGAAAACCCCGGGCCTGCAT |
| | | GCATGTACAACATGATGGAGACGGAGCTGAAGCCGCCGGGCCCGCAGCAA |
| | | ACTTCGGGGGGCGGCGGCGGCAACTCCACCGCGGCGGCGGCCGGCGGCAA |
| | | CCAGAAAAACAGCCCGGACCGCGTCAAGCGGCCCATGAATGCCTTCATGG |
| | | TGTGGTCCCGCGGGCAGCGGCGCAAGATGGCCCAGGAGAACCCCAAGATG |
| | | CACAACTCGGAGATCAGCAAGCGCCTGGGCGCCGAGTGGAAACTTTTGTC |
| | | GGAGACGGAGAAGCGGCCGTTCATCGACGAGGCTAAGCGGCTGCGAGCGC |
| | | TGCACATGAAGGAGCACCCGGATTATAAATACCGGCCCCGGCGGAAAACC |
| | | AAGACGCTCATGAAGAAGGATAAGTACACGCTGCCCGGCGGGCTGCTGGC |
| | | CCCCGGCGGCAATAGCATGGCGAGCGGGGTCGGGGTGGGCGCCGGCCTGG |
| | | GCGCGGGCGTGAACCAGCGCATGGACAGTTACGCGCACATGAACGGCTGG |
| | | AGCAACGGCAGCTACAGCATGATGCAGGACCAGCTGGGCTACCCGCAGCA |
| | | CCCGGGCCTCAATGCGCACGGCGCAGCGCAGATGCAGCCCATGCACCGCT |
| | | ACGACGTGAGCGCCCTGCAGTACAACTCCATGACCAGCTCGCAGACCTAC |
| | | ATGAACGGCTCGCCCACCTACAGCATGTCCTACTCGCAGCAGGGCACCCC |
| | | TGGCATGGCTCTTGGCTCCATGGGTTCGGTGGTCAAGTCCGAGGCCAGCT |
| | | CCAGCCCCCCTGTGGTTACCTCTTCCTCCCACTCCAGGGCGCCCTGCCAG |
| | | GCCGGGGACCTCCGGGACATGATCAGCATGTATCTCCCCGGCGCCGAGGT |
| | | GCCGGAACCCGCCGCCCCCAGCAGACTTCACATGTCCCAGCACTACCAGA |
| | | GCGGCCCGGTGCCCGGCACGGCCATTAACGGCACACTGCCCCTCTCACAC |
| | | ATGGCATGCGGCTCCGGCGAGGGCAGGGGAAGTCTTCTAACATGCGGGGA |
| | | CGTGGAGGAAAATCCCGGCCCACTCGAGATGGCTGTCAGCGACGCGCTGC |
| | | TCCCATCTTTCTCCACGTTCGCGTCTGGCCCGGCGGGAAGGGAGAAGACA |
| | | CTGCGTCAAGCAGGTGCCCCGAATAACCGCTGGCGGGAGGAGCTCTCCCA |
| | | CATGAAGCGACTTCCCCCAGTGCTTCCCGGCCGCCCCTATGACCTGGCGG |
| | | CGGCGACCGTGGCCACAGACCTGGAGAGCGGCGGAGCCGGTGCGGCTTGC |
| | | GGCGGTAGCAACCTGGCGCCCCTACCTCGGAGAGAGACCGAGGAGTTCAA |
| | | CGATCTCCTGGACCTGGACTTTATTCTCTCCAATTCGCTGACCCATCCTC |
| | | CGGAGTCAGTGGCCGCCACCGTGTCCTCGTCAGCGTCAGCCTCCTCTTCG |
| | | TCGTCGCCGTCGAGCAGCGGCCCTGCCAGCGCGCCCTCCACCTGCAGCTT |
| | | CACCTATCCGATCCGGGCCGGGAACGACCCGGGCGTGGCGCCGGGCGGCA |
| | | CGGGCGGAGGCCTCCTCTATGGCAGGGAGTCCGCTCCCCCTCCGACGGCT |
| | | CCCTTCAACCTGGCGGACATCAACGACGTGAGCCCCTCGGGCGGCTTCGT |
| | | GGCCGAGCTCCTGCGGCCAGAATTGGACCCGGTGTACATTCCGCCGCAGC |
| | | AGCCGCAGCCGCCAGGTGGCGGGCTGATGGGCAAGTTCGTGCTGAAGGCG |
| | | TCGCTGAGCGCCCCTGGCAGCGAGTACGGCAGCCCGTCGGTCATCAGCGT |
| | | CAGCAAAGGCAGCCCTGACGGCAGCCACCCGGTGGTGGTGGCGCCCTACA |
| | | ACGGCGGGCCGCCGCGCACGTGCCCCAAGATCAAGCAGGAGGCGGTCTCT |
| | | TCGTGCACCCACTTGGGCGCTGGACCCCCTCTCAGCAATGGCCACCGGCC |
| | | GGCTGCACACGACTTCCCCCTGGGGCGGCAGCTCCCCAGCAGGACTACCC |
| | | CGACCCTGGGTCTTGAGGAAGTGCTGAGCAGCAGGGACTGTCACCCTGCC |
| | | CTGCCGCTTCCTCCCGGCTTCCATCCCCACCCGGGGCCCAATTACCCATC |
| | | CTTCCTGCCCGATCAGATGCAGCCGCAAGTCCCGCCGCTCCATTACCAAG |
| | | AGCTCATGCCACCCGGTTCCTGCATGCCAGAGGAGCCCAAGCCAAAGAGG |
| | | GGAAGACGATCGTGGCCCCGGAAAAGGACCGCCACCCACACTTGTGATTA |
| | | CGCGGGCTGCGGCAAAACCTACACAAAGAGTTCCCATCTCAAGGCACACC |
| | | TGCGAACCCACACAGGTGAGAAACCTTACCACTGTGACTGGGACGGCTGT |
| | | GGATGGAAATTCGCCCGCTCAGATGAACTGACCAGGCACTACCGTAAACA |
| | | CACGGGGCACCGCCCGTTCCAGTGCCAAAAATGCGACCGAGCATTTTCCA |
| | | GGTCGGACCACCTCGCCTTACACATGAAGAGGCATTTTTAAATGACTAGT |
| | | GCGCGCAGCGGCCGACCATGGCCCAACTTGTTTATTGCAGCTTATAATGG |
| | | TTACAAATAAAGCAATAGCATCACAAATTTCACAAATAAAGCATTTTTTT |
| | | CACTGCATTCTAGTTGTGGTTTGTCCAAACTCATCAATGTATCTTATCAT |
| | | GTCTGGATCTCGGTACCGGATCCAAATTCCCGATAAGGATCTTCCTAGAG |
| | | CATGGCTACGTAGATAAGTAGCATGGCGGGTTAATCATTAACTACAAGGA |
| | | ACCCCTAGTGATGGAGTTGGCCACTCCCTCTCTGCGCGCTCGCTCGCTCA |
| | | CTGAGGCCGGGCGACCAAAGGTCGCCCGACGCCCGGGCTTTGCCCGGGCG |
| | | GCCTCAGTGAGCGAGCGAGCGCGCAGCCTTAATTAACCTAATTCATCCCA |
| | | ATGGCGCGCCGAGCTTGGCTCGAGCATGGTCAT |
| 14 | OCT4-2A- | ATGGCGGGACACCTGGCTTCGGATTTCGCCTTCTCGCCCCCTCCAGGTGG |
| | SOX2-2A- | TGGAGGTGATGGGCCAGGGGGGCCGGAGCCGGGCTGGGTTGATCCTCGGA |
| | KLF4 (Whole | CCTGGCTAAGCTTCCAAGGCCCTCCTGGAGGGCCAGGAATCGGGCCGGGG |
| | insert sequence, | GTTGGGCCAGGCTCTGAGGTGTGGGGGATTCCCCCATGCCCCCCGCCGTA |
| | does not include | TGAGTTCTGTGGGGGGATGGCGTACTGTGGGCCCCAGGTTGGAGTGGGGC |
| | SV40 or Tet | TAGTGCCCCAAGGCGGCTTGGAGACCTCTCAGCCTGAGGGCGAAGCAGGA |
| | Operators) | GTCGGGGTGGAGAGCAACTCCGATGGGGCCTCCCCGGAGCCCTGCACCGT |
| | | CACCCCTGGTGCCGTGAAGCTGGAGAAGGAGAAGCTGGAGCAAAACCCGG |
| | | AGGAGTCCCAGGACATCAAAGCTCTGCAGAAAGAACTCGAGCAATTTGCC |
| | | AAGCTCCTGAAGCAGAAGAGGATCACCCTGGGATATACACAGGCCGATGT |
| | | GGGGCTCACCCTGGGGGTTCTATTTGGGAAGGTATTCAGCCAAACGACCA |
| | | TCTGCCGCTTTGAGGCTCTGCAGCTTAGCTTCAAGAACATGTGTAAGCTG |
| | | CGGCCCTTGCTGCAGAAGTGGGTGGAGGAAGCTGACAACAATGAAAATCT |
| | | TCAGGAGATATGCAAAGCAGAAACCCTCGTGCAGGCCCGAAAGAGAAAGC |
| | | GAACCAGTATCGAGAACCGAGTGAGAGGCAACCTGGAGAATTTGTTCCTG |
| | | CAGTGCCCGAAACCCACACTGCAGCAGATCAGCCACATCGCCCAGCAGCT |
| | | TGGGCTCGAGAAGGATGTGGTCCGAGTGTGGTTCTGTAACCGGCGCCAGA |
| | | AGGGCAAGCGATCAAGCAGCGACTATGCACAACGAGAGGATTTTGAGGCT |
| | | GCTGGGTCTCCTTTCTCAGGGGGACCAGTGTCCTTTCCTCTGGCCCCAGG |
| | | GCCCCATTTTGGTACCCCAGGCTATGGGAGCCCTCACTTCACTGCACTGT |
| | | ACTCCTCGGTCCCTTTCCCTGAGGGGGAAGCCTTTCCCCCTGTCTCTGTC |
| | | ACCACTCTGGGCTCTCCCATGCATTCAAACGCTAGCGGCAGCGGCGCCAC |
| | | GAACTTCTCTCTGTTAAAGCAAGCAGGAGATGTTGAAGAAAACCCCGGGC |
| | | CTGCATGCATGTACAACATGATGGAGACGGAGCTGAAGCCGCCGGGCCCG |
| | | CAGCAAACTTCGGGGGGCGGCGGCGGCAACTCCACCGCGGCGGCGGCCGG |
| | | CGGCAACCAGAAAAACAGCCCGGACCGCGTCAAGCGGCCCATGAATGCCT |
| | | TCATGGTGTGGTCCCGCGGGCAGCGGCGCAAGATGGCCCAGGAGAACCCC |
| | | AAGATGCACAACTCGGAGATCAGCAAGCGCCTGGGCGCCGAGTGGAAACT |
| | | TTTGTCGGAGACGGAGAAGCGGCCGTTCATCGACGAGGCTAAGCGGCTGC |
| | | GAGCGCTGCACATGAAGGAGCACCCGGATTATAAATACCGGCCCCGGCGG |
| | | AAAACCAAGACGCTCATGAAGAAGGATAAGTACACGCTGCCCGGCGGGCT |
| | | GCTGGCCCCCGGCGGCAATAGCATGGCGAGCGGGGTCGGGGTGGGCGCCG |
| | | GCCTGGGCGCGGGCGTGAACCAGCGCATGGACAGTTACGCGCACATGAAC |
| | | GGCTGGAGCAACGGCAGCTACAGCATGATGCAGGACCAGCTGGGCTACCC |
| | | GCAGCACCCGGGCCTCAATGCGCACGGCGCAGCGCAGATGCAGCCCATGC |
| | | ACCGCTACGACGTGAGCGCCCTGCAGTACAACTCCATGACCAGCTCGCAG |
| | | ACCTACATGAACGGCTCGCCCACCTACAGCATGTCCTACTCGCAGCAGGG |
| | | CACCCCTGGCATGGCTCTTGGCTCCATGGGTTCGGTGGTCAAGTCCGAGG |
| | | CCAGCTCCAGCCCCCCTGTGGTTACCTCTTCCTCCCACTCCAGGGCGCCC |
| | | TGCCAGGCCGGGGACCTCCGGGACATGATCAGCATGTATCTCCCCGGCGC |
| | | CGAGGTGCCGGAACCCGCCGCCCCCAGCAGACTTCACATGTCCCAGCACT |
| | | ACCAGAGCGGCCCGGTGCCCGGCACGGCCATTAACGGCACACTGCCCCTC |
| | | TCACACATGGCATGCGGCTCCGGCGAGGGCAGGGGAAGTCTTCTAACATG |
| | | CGGGGACGTGGAGGAAAATCCCGGCCCACTCGAGATGGCTGTCAGCGACG |
| | | CGCTGCTCCCATCTTTCTCCACGTTCGCGTCTGGCCCGGCGGGAAGGGAG |
| | | AAGACACTGCGTCAAGCAGGTGCCCCGAATAACCGCTGGCGGGAGGAGCT |
| | | CTCCCACATGAAGCGACTTCCCCCAGTGCTTCCCGGCCGCCCCTATGACC |
| | | TGGCGGCGGCGACCGTGGCCACAGACCTGGAGAGCGGCGGAGCCGGTGCG |
| | | GCTTGCGGCGGTAGCAACCTGGCGCCCCTACCTCGGAGAGAGACCGAGGA |
| | | GTTCAACGATCTCCTGGACCTGGACTTTATTCTCTCCAATTCGCTGACCC |
| | | ATCCTCCGGAGTCAGTGGCCGCCACCGTGTCCTCGTCAGCGTCAGCCTCC |
| | | TCTTCGTCGTCGCCGTCGAGCAGCGGCCCTGCCAGCGCGCCCTCCACCTG |
| | | CAGCTTCACCTATCCGATCCGGGCCGGGAACGACCCGGGCGTGGCGCCGG |
| | | GCGGCACGGGCGGAGGCCTCCTCTATGGCAGGGAGTCCGCTCCCCCTCCG |
| | | ACGGCTCCCTTCAACCTGGCGGACATCAACGACGTGAGCCCCTCGGGCGG |
| | | CTTCGTGGCCGAGCTCCTGCGGCCAGAATTGGACCCGGTGTACATTCCGC |
| | | CGCAGCAGCCGCAGCCGCCAGGTGGCGGGCTGATGGGCAAGTTCGTGCTG |
| | | AAGGCGTCGCTGAGCGCCCCTGGCAGCGAGTACGGCAGCCCGTCGGTCAT |
| | | CAGCGTCAGCAAAGGCAGCCCTGACGGCAGCCACCCGGTGGTGGTGGCGC |
| | | CCTACAACGGCGGGCCGCCGCGCACGTGCCCCAAGATCAAGCAGGAGGCG |
| | | GTCTCTTCGTGCACCCACTTGGGCGCTGGACCCCCTCTCAGCAATGGCCA |
| | | CCGGCCGGCTGCACACGACTTCCCCCTGGGGCGGCAGCTCCCCAGCAGGA |
| | | CTACCCCGACCCTGGGTCTTGAGGAAGTGCTGAGCAGCAGGGACTGTCAC |
| | | CCTGCCCTGCCGCTTCCTCCCGGCTTCCATCCCCACCCGGGGCCCAATTA |
| | | CCCATCCTTCCTGCCCGATCAGATGCAGCCGCAAGTCCCGCCGCTCCATT |
| | | ACCAAGAGCTCATGCCACCCGGTTCCTGCATGCCAGAGGAGCCCAAGCCA |
| | | AAGAGGGGAAGACGATCGTGGCCCCGGAAAAGGACCGCCACCCACACTTG |
| | | TGATTACGCGGGCTGCGGCAAAACCTACACAAAGAGTTCCCATCTCAAGG |
| | | CACACCTGCGAACCCACACAGGTGAGAAACCTTACCACTGTGACTGGGAC |
| | | GGCTGTGGATGGAAATTCGCCCGCTCAGATGAACTGACCAGGCACTACCG |
| | | TAAACACACGGGGCACCGCCCGTTCCAGTGCCAAAAATGCGACCGAGCAT |
| | | TTTCCAGGTCGGACCACCTCGCCTTACACATGAAGAGGCATTTT |
| 15 | pAAV2- | TCGCGCGTTTCGGTGATGACGGTGAAAACCTCTGACACATGCAGCTCCCG |
| | TRE3G-OSK- | GAGACTGTCACAGCTTGTCTGTAAGCGGATGCCGGGAGCAGACAAGCCCG |
| | SV40 (full | TCAGGGCGCGTCAGCGGGTGTTGGCGGGTGTCGGGGCTGGCTTAACTATG |
| | plasmid | CGGCATCAGAGCAGATTGTACTGAGAGTGCACCATATGCGGTGTGAAATA |
| | sequence, | CCGCACAGATGCGTAAGGAGAAAATACCGCATCAGGCGCCATTCGCCATT |
| | including | CAGGCTGCGCAACTGTTGGGAAGGGCGATCGGTGCGGGCCTCTTCGCTAT |
| | selection | TACGCCAGCTGGCGAAAGGGGGATGTGCTGCAAGGCGATTAAGTTGGGTA |
| | casettes) | ACGCCAGGGTTTTCCCAGTCACGACGTTGTAAAACGACGGCCAGTGAATT |
| | | GACGCGTATTGGGATCCATGATTACGCCAGATTTAATTAAGGCCTTAATT |
| | | AGGCTGCGCGCTCGCTCGCTCACTGAGGCCGCCCGGGCAAAGCCCGGGCG |
| | | TCGGGCGACCTTTGGTCGCCCGGCCTCAGTGAGCGAGCGAGCGCGCAGAG |
| | | AGGGAGTGGCCAACTCCATCACTAGGGGTTCCTTGTAGTTAATGATTAAC |
| | | CCGCCATGCTACTTATCTACGTAGCCATGCTCTAGGAAGATCGGAATTCT |
| | | TTACTCCCTATCAGTGATAGAGAACGTATGAAGAGTTTACTCCCTATCAG |
| | | TGATAGAGAACGTATGCAGACTTTACTCCCTATCAGTGATAGAGAACGTA |
| | | TAAGGAGTTTACTCCCTATCAGTGATAGAGAACGTATGACCAGTTTACTC |
| | | CCTATCAGTGATAGAGAACGTATCTACAGTTTACTCCCTATCAGTGATAG |
| | | AGAACGTATATCCAGTTTACTCCCTATCAGTGATAGAGAACGTATAAGCT |
| | | TTAGGCGTGTACGGTGGGCGCCTATAAAAGCAGAGCTCGTTTAGTGAACC |
| | | GTCAGATCGCCTGGAGCAATTCCACAACACTTTTGTCTTATACCAACTTT |
| | | CCGTACCACTTCCTACCCTCGTAAAGCGGCCGCGCCACCATGGCGGGACA |
| | | CCTGGCTTCGGATTTCGCCTTCTCGCCCCCTCCAGGTGGTGGAGGTGATG |
| | | GGCCAGGGGGGCCGGAGCCGGGCTGGGTTGATCCTCGGACCTGGCTAAGC |
| | | TTCCAAGGCCCTCCTGGAGGGCCAGGAATCGGGCCGGGGGTTGGGCCAGG |
| | | CTCTGAGGTGTGGGGGATTCCCCCATGCCCCCCGCCGTATGAGTTCTGTG |
| | | GGGGGATGGCGTACTGTGGGCCCCAGGTTGGAGTGGGGCTAGTGCCCCAA |
| | | GGCGGCTTGGAGACCTCTCAGCCTGAGGGCGAAGCAGGAGTCGGGGTGGA |
| | | GAGCAACTCCGATGGGGCCTCCCCGGAGCCCTGCACCGTCACCCCTGGTG |
| | | CCGTGAAGCTGGAGAAGGAGAAGCTGGAGCAAAACCCGGAGGAGTCCCAG |
| | | GACATCAAAGCTCTGCAGAAAGAACTCGAGCAATTTGCCAAGCTCCTGAA |
| | | GCAGAAGAGGATCACCCTGGGATATACACAGGCCGATGTGGGGCTCACCC |
| | | TGGGGGTTCTATTTGGGAAGGTATTCAGCCAAACGACCATCTGCCGCTTT |
| | | GAGGCTCTGCAGCTTAGCTTCAAGAACATGTGTAAGCTGCGGCCCTTGCT |
| | | GCAGAAGTGGGTGGAGGAAGCTGACAACAATGAAAATCTTCAGGAGATAT |
| | | GCAAAGCAGAAACCCTCGTGCAGGCCCGAAAGAGAAAGCGAACCAGTATC |
| | | GAGAACCGAGTGAGAGGCAACCTGGAGAATTTGTTCCTGCAGTGCCCGAA |
| | | ACCCACACTGCAGCAGATCAGCCACATCGCCCAGCAGCTTGGGCTCGAGA |
| | | AGGATGTGGTCCGAGTGTGGTTCTGTAACCGGCGCCAGAAGGGCAAGCGA |
| | | TCAAGCAGCGACTATGCACAACGAGAGGATTTTGAGGCTGCTGGGTCTCC |
| | | TTTCTCAGGGGGACCAGTGTCCTTTCCTCTGGCCCCAGGGCCCCATTTTG |
| | | GTACCCCAGGCTATGGGAGCCCTCACTTCACTGCACTGTACTCCTCGGTC |
| | | CCTTTCCCTGAGGGGGAAGCCTTTCCCCCTGTCTCTGTCACCACTCTGGG |
| | | CTCTCCCATGCATTCAAACGCTAGCGGCAGCGGCGCCACGAACTTCTCTC |
| | | TGTTAAAGCAAGCAGGAGATGTTGAAGAAAACCCCGGGCCTGCATGCATG |
| | | TACAACATGATGGAGACGGAGCTGAAGCCGCCGGGCCCGCAGCAAACTTC |
| | | GGGGGGCGGCGGCGGCAACTCCACCGCGGCGGCGGCCGGCGGCAACCAGA |
| | | AAAACAGCCCGGACCGCGTCAAGCGGCCCATGAATGCCTTCATGGTGTGG |
| | | TCCCGCGGGCAGCGGCGCAAGATGGCCCAGGAGAACCCCAAGATGCACAA |
| | | CTCGGAGATCAGCAAGCGCCTGGGCGCCGAGTGGAAACTTTTGTCGGAGA |
| | | CGGAGAAGCGGCCGTTCATCGACGAGGCTAAGCGGCTGCGAGCGCTGCAC |
| | | ATGAAGGAGCACCCGGATTATAAATACCGGCCCCGGCGGAAAACCAAGAC |
| | | GCTCATGAAGAAGGATAAGTACACGCTGCCCGGCGGGCTGCTGGCCCCCG |
| | | GCGGCAATAGCATGGCGAGCGGGGTCGGGGTGGGCGCCGGCCTGGGCGCG |
| | | GGCGTGAACCAGCGCATGGACAGTTACGCGCACATGAACGGCTGGAGCAA |
| | | CGGCAGCTACAGCATGATGCAGGACCAGCTGGGCTACCCGCAGCACCCGG |
| | | GCCTCAATGCGCACGGCGCAGCGCAGATGCAGCCCATGCACCGCTACGAC |
| | | GTGAGCGCCCTGCAGTACAACTCCATGACCAGCTCGCAGACCTACATGAA |
| | | CGGCTCGCCCACCTACAGCATGTCCTACTCGCAGCAGGGCACCCCTGGCA |
| | | TGGCTCTTGGCTCCATGGGTTCGGTGGTCAAGTCCGAGGCCAGCTCCAGC |
| | | CCCCCTGTGGTTACCTCTTCCTCCCACTCCAGGGCGCCCTGCCAGGCCGG |
| | | GGACCTCCGGGACATGATCAGCATGTATCTCCCCGGCGCCGAGGTGCCGG |
| | | AACCCGCCGCCCCCAGCAGACTTCACATGTCCCAGCACTACCAGAGCGGC |
| | | CCGGTGCCCGGCACGGCCATTAACGGCACACTGCCCCTCTCACACATGGC |
| | | ATGCGGCTCCGGCGAGGGCAGGGGAAGTCTTCTAACATGCGGGGACGTGG |
| | | AGGAAAATCCCGGCCCACTCGAGATGGCTGTCAGCGACGCGCTGCTCCCA |
| | | TCTTTCTCCACGTTCGCGTCTGGCCCGGCGGGAAGGGAGAAGACACTGCG |
| | | TCAAGCAGGTGCCCCGAATAACCGCTGGCGGGAGGAGCTCTCCCACATGA |
| | | AGCGACTTCCCCCAGTGCTTCCCGGCCGCCCCTATGACCTGGCGGCGGCG |
| | | ACCGTGGCCACAGACCTGGAGAGCGGCGGAGCCGGTGCGGCTTGCGGCGG |
| | | TAGCAACCTGGCGCCCCTACCTCGGAGAGAGACCGAGGAGTTCAACGATC |
| | | TCCTGGACCTGGACTTTATTCTCTCCAATTCGCTGACCCATCCTCCGGAG |
| | | TCAGTGGCCGCCACCGTGTCCTCGTCAGCGTCAGCCTCCTCTTCGTCGTC |
| | | GCCGTCGAGCAGCGGCCCTGCCAGCGCGCCCTCCACCTGCAGCTTCACCT |
| | | ATCCGATCCGGGCCGGGAACGACCCGGGCGTGGCGCCGGGCGGCACGGGC |
| | | GGAGGCCTCCTCTATGGCAGGGAGTCCGCTCCCCCTCCGACGGCTCCCTT |
| | | CAACCTGGCGGACATCAACGACGTGAGCCCCTCGGGCGGCTTCGTGGCCG |
| | | AGCTCCTGCGGCCAGAATTGGACCCGGTGTACATTCCGCCGCAGCAGCCG |
| | | CAGCCGCCAGGTGGCGGGCTGATGGGCAAGTTCGTGCTGAAGGCGTCGCT |
| | | GAGCGCCCCTGGCAGCGAGTACGGCAGCCCGTCGGTCATCAGCGTCAGCA |
| | | AAGGCAGCCCTGACGGCAGCCACCCGGTGGTGGTGGCGCCCTACAACGGC |
| | | GGGCCGCCGCGCACGTGCCCCAAGATCAAGCAGGAGGCGGTCTCTTCGTG |
| | | CACCCACTTGGGCGCTGGACCCCCTCTCAGCAATGGCCACCGGCCGGCTG |
| | | CACACGACTTCCCCCTGGGGCGGCAGCTCCCCAGCAGGACTACCCCGACC |
| | | CTGGGTCTTGAGGAAGTGCTGAGCAGCAGGGACTGTCACCCTGCCCTGCC |
| | | GCTTCCTCCCGGCTTCCATCCCCACCCGGGGCCCAATTACCCATCCTTCC |
| | | TGCCCGATCAGATGCAGCCGCAAGTCCCGCCGCTCCATTACCAAGAGCTC |
| | | ATGCCACCCGGTTCCTGCATGCCAGAGGAGCCCAAGCCAAAGAGGGGAAG |
| | | ACGATCGTGGCCCCGGAAAAGGACCGCCACCCACACTTGTGATTACGCGG |
| | | GCTGCGGCAAAACCTACACAAAGAGTTCCCATCTCAAGGCACACCTGCGA |
| | | ACCCACACAGGTGAGAAACCTTACCACTGTGACTGGGACGGCTGTGGATG |
| | | GAAATTCGCCCGCTCAGATGAACTGACCAGGCACTACCGTAAACACACGG |
| | | GGCACCGCCCGTTCCAGTGCCAAAAATGCGACCGAGCATTTTCCAGGTCG |
| | | GACCACCTCGCCTTACACATGAAGAGGCATTTTTAAATGACTAGTGCGCG |
| | | CAGCGGCCGACCATGGCCCAACTTGTTTATTGCAGCTTATAATGGTTACA |
| | | AATAAAGCAATAGCATCACAAATTTCACAAATAAAGCATTTTTTTCACTG |
| | | CATTCTAGTTGTGGTTTGTCCAAACTCATCAATGTATCTTATCATGTCTG |
| | | GATCTCGGTACCGGATCCAAATTCCCGATAAGGATCTTCCTAGAGCATGG |
| | | CTACGTAGATAAGTAGCATGGCGGGTTAATCATTAACTACAAGGAACCCC |
| | | TAGTGATGGAGTTGGCCACTCCCTCTCTGCGCGCTCGCTCGCTCACTGAG |
| | | GCCGGGCGACCAAAGGTCGCCCGACGCCCGGGCTTTGCCCGGGCGGCCTC |
| | | AGTGAGCGAGCGAGCGCGCAGCCTTAATTAACCTAATTCATCCCAATGGC |
| | | GCGCCGAGCTTGGCTCGAGCATGGTCATAGCTGTTTCCTGTGTGAAATTG |
| | | TTATCCGCTCACAATTCCACACAACATACGAGCCGGAAGCATAAAGTGTA |
| | | AAGCCTGGGGTGCCTAATGAGTGAGCTAACTCACATTAATTGCGTTGCGC |
| | | TCACTGCCCGCTTTCCAGTCGGGAAACCTGTCGTGCCAGCTGCATTAATG |
| | | AATCGGCCAACGCGCGGGGAGAGGCGGTTTGCGTATTGGGCGCTGTTCCG |
| | | CTTCCTCGCTCACTGACTCGCTGCGCTCGGTCGTTCGGCTGCGGCGAGCG |
| | | GTATCAGCTCACTCAAAGGCGGTAATACGGTTATCCACAGAATCAGGGGA |
| | | TAACGCAGGAAAGAACATGTGAGCAAAAGGCCAGCAAAAGGCCAGGAACC |
| | | GTAAAAAGGCCGCGTTGCTGGCGTTTTTCCATAGGCTCCGCCCCCCTGAC |
| | | GAGCATCACAAAAATCGACGCTCAAGTCAGAGGTGGCGAAACCCGACAGG |
| | | ACTATAAAGATACCAGGCGTTTCCCCCTGGAAGCTCCCTCGTGCGCTCTC |
| | | CTGTTCCGACCCTGCCGCTTACCGGATACCTGTCCGCCTTTCTCCCTTCG |
| | | GGAAGCGTGGCGCTTTCTCATAGCTCACGCTGTAGGTATCTCAGTTCGGT |
| | | GTAGGTCGTTCGCTCCAAGCTGGGCTGTGTGCACGAACCCCCCGTTCAGC |
| | | CCGACCGCTGCGCCTTATCCGGTAACTATCGTCTTGAGTCCAACCCGGTA |
| | | AGACACGACTTATCGCCACTGGCAGCAGCCACTGGTAACAGGATTAGCAG |
| | | AGCGAGGTATGTAGGCGGTGCTACAGAGTTCTTGAAGTGGTGGCCTAACT |
| | | ACGGCTACACTAGAAGAACAGTATTTGGTATCTGCGCTCTGCTGAAGCCA |
| | | GTTACCTTCGGAAAAAGAGTTGGTAGCTCTTGATCCGGCAAACAAACCAC |
| | | CGCTGGTAGCGGTGGTTTTTTTGTTTGCAAGCAGCAGATTACGCGCAGAA |
| | | AAAAAGGATCTCAAGAAGATCCTTTGATCTTTTCTACGGGGTCTGACGCT |
| | | CAGTGGAACGAAAACTCACGTTAAGGGATTTTGGTCATGAGATTATCAAA |
| | | AAGGATCTTCACCTAGATCCTTTTAAATTAAAAATGAAGTTTTAAATCAA |
| | | TCTAAAGTATATATGAGTAAACTTGGTCTGACAGTTAGAAAAACTCATCG |
| | | AGCATCAAATGAAACTGCAATTTATTCATATCAGGATTATCAATACCATA |
| | | TTTTTGAAAAAGCCGTTTCTGTAATGAAGGAGAAAACTCACCGAGGCAGT |
| | | TCCATAGGATGGCAAGATCCTGGTATCGGTCTGCGATTCCGACTCGTCCA |
| | | ACATCAATACAACCTATTAATTTCCCCTCGTCAAAAATAAGGTTATCAAG |
| | | TGAGAAATCACCATGAGTGACGACTGAATCCGGTGAGAATGGCAAAAGTT |
| | | TATGCATTTCTTTCCAGACTTGTTCAACAGGCCAGCCATTACGCTCGTCA |
| | | TCAAAATCACTCGCATCAACCAAACCGTTATTCATTCGTGATTGCGCCTG |
| | | AGCGAAACGAAATACGCGATCGCTGTTAAAAGGACAATTACAAACAGGAA |
| | | TCGAATGCAACCGGCGCAGGAACACTGCCAGCGCATCAACAATATTTTCA |
| | | CCTGAATCAGGATATTCTTCTAATACCTGGAATGCTGTTTTCCCAGGGAT |
| | | CGCAGTGGTGAGTAACCATGCATCATCAGGAGTACGGATAAAATGCTTGA |
| | | TGGTCGGAAGAGGCATAAATTCCGTCAGCCAGTTTAGTCTGACCATCTCA |
| | | TCTGTAACATCATTGGCAACGCTACCTTTGCCATGTTTCAGAAACAACTC |
| | | TGGCGCATCGGGCTTCCCATACAATCGATAGATTGTCGCACCTGATTGCC |
| | | CGACATTATCGCGAGCCCATTTATACCCATATAAATCAGCATCCATGTTG |
| | | GAATTTAATCGCGGCCTAGAGCAAGACGTTTCCCGTTGAATATGGCTCAT |
| | | ACTCTTCCTTTTTCAATATTATTGAAGCATTTATCAGGGTTATTGTCTCA |
| | | TGAGCGGATACATATTTGAATGTATTTAGAAAAATAAACAAATAGGGGTT |
| | | CCGCGCACATTTCCCCGAAAAGTGCCACCTGACGTCTAAGAAACCATTAT |
| | | TATCATGACATTAACCTATAAAAATAGGCGTATCACGAGGCCCTTTTGTC |
| 16 | ITR - forward | CCTTAATTAGGCTGCGCGCTCGCTCGCTCACTGAGGCCGCCCGGGCAAAG |
| | | CCCGGGCGTCGGGCGACCTTTGGTCGCCCGGCCTCAGTGAGCGAGCGAGC |
| | | GCGCAGAGAGGGAGTGGCCAACTCCATCACTAGGGGTTCCT |
| 32 | ITR - reverse | AGGAACCCCTAGTGATGGAGTTGGCCACTCCCTCTCTGCGCGCTCGCTCG |
| | | CTCACTGAGGCCGGGCGACCAAAGGTCGCCCGACGCCCGGGCTTTGCCCG |
| | | GGCGGCCTCAGTGAGCGAGCGAGCGCGCAGCTGCCTGCAGG |
| 35 | OCT4-2A- | CCTTAATTAGGCTGCGCGCTCGCTCGCTCACTGAGGCCGCCCGGGCAAAG |
| | SOX2-2A- | CCCGGGCGTCGGGCGACCTTTGGTCGCCCGGCCTCAGTGAGCGAGCGAGC |
| | KLF4 (Whole | GCGCAGAGAGGGAGTGGCCAACTCCATCACTAGGGGTTCCTTGTAGTTAA |
| | insert sequence | TGATTAACCCGCCATGCTACTTATCTACGTAGCCATGCTCTAGGAAGATC |
| | including Tet | GGAATTCTTTACTCCCTATCAGTGATAGAGAACGTATGAAGAGTTTACTC |
| | operators, | CCTATCAGTGATAGAGAACGTATGCAGACTTTACTCCCTATCAGTGATAG |
| | SV40, and | AGAACGTATAAGGAGTTTACTCCCTATCAGTGATAGAGAACGTATGACCA |
| | ITRs) | GTTTACTCCCTATCAGTGATAGAGAACGTATCTACAGTTTACTCCCTATC |
| | | AGTGATAGAGAACGTATATCCAGTTTACTCCCTATCAGTGATAGAGAACG |
| | | TATAAGCTTTAGGCGTGTACGGTGGGCGCCTATAAAAGCAGAGCTCGTTT |
| | | AGTGAACCGTCAGATCGCCTGGAGCAATTCCACAACACTTTTGTCTTATA |
| | | CCAACTTTCCGTACCACTTCCTACCCTCGTAAAGCGGCCGCGCCACCATG |
| | | GCGGGACACCTGGCTTCGGATTTCGCCTTCTCGCCCCCTCCAGGTGGTGG |
| | | AGGTGATGGGCCAGGGGGGCCGGAGCCGGGCTGGGTTGATCCTCGGACCT |
| | | GGCTAAGCTTCCAAGGCCCTCCTGGAGGGCCAGGAATCGGGCCGGGGGTT |
| | | GGGCCAGGCTCTGAGGTGTGGGGGATTCCCCCATGCCCCCCGCCGTATGA |
| | | GTTCTGTGGGGGGATGGCGTACTGTGGGCCCCAGGTTGGAGTGGGGCTAG |
| | | TGCCCCAAGGCGGCTTGGAGACCTCTCAGCCTGAGGGCGAAGCAGGAGTC |
| | | GGGGTGGAGAGCAACTCCGATGGGGCCTCCCCGGAGCCCTGCACCGTCAC |
| | | CCCTGGTGCCGTGAAGCTGGAGAAGGAGAAGCTGGAGCAAAACCCGGAGG |
| | | AGTCCCAGGACATCAAAGCTCTGCAGAAAGAACTCGAGCAATTTGCCAAG |
| | | CTCCTGAAGCAGAAGAGGATCACCCTGGGATATACACAGGCCGATGTGGG |
| | | GCTCACCCTGGGGGTTCTATTTGGGAAGGTATTCAGCCAAACGACCATCT |
| | | GCCGCTTTGAGGCTCTGCAGCTTAGCTTCAAGAACATGTGTAAGCTGCGG |
| | | CCCTTGCTGCAGAAGTGGGTGGAGGAAGCTGACAACAATGAAAATCTTCA |
| | | GGAGATATGCAAAGCAGAAACCCTCGTGCAGGCCCGAAAGAGAAAGCGAA |
| | | CCAGTATCGAGAACCGAGTGAGAGGCAACCTGGAGAATTTGTTCCTGCAG |
| | | TGCCCGAAACCCACACTGCAGCAGATCAGCCACATCGCCCAGCAGCTTGG |
| | | GCTCGAGAAGGATGTGGTCCGAGTGTGGTTCTGTAACCGGCGCCAGAAGG |
| | | GCAAGCGATCAAGCAGCGACTATGCACAACGAGAGGATTTTGAGGCTGCT |
| | | GGGTCTCCTTTCTCAGGGGGACCAGTGTCCTTTCCTCTGGCCCCAGGGCC |
| | | CCATTTTGGTACCCCAGGCTATGGGAGCCCTCACTTCACTGCACTGTACT |
| | | CCTCGGTCCCTTTCCCTGAGGGGGAAGCCTTTCCCCCTGTCTCTGTCACC |
| | | ACTCTGGGCTCTCCCATGCATTCAAACGCTAGCGGCAGCGGCGCCACGAA |
| | | CTTCTCTCTGTTAAAGCAAGCAGGAGATGTTGAAGAAAACCCCGGGCCTG |
| | | CATGCATGTACAACATGATGGAGACGGAGCTGAAGCCGCCGGGCCCGCAG |
| | | CAAACTTCGGGGGGCGGCGGCGGCAACTCCACCGCGGCGGCGGCCGGCGG |
| | | CAACCAGAAAAACAGCCCGGACCGCGTCAAGCGGCCCATGAATGCCTTCA |
| | | TGGTGTGGTCCCGCGGGCAGCGGCGCAAGATGGCCCAGGAGAACCCCAAG |
| | | ATGCACAACTCGGAGATCAGCAAGCGCCTGGGCGCCGAGTGGAAACTTTT |
| | | GTCGGAGACGGAGAAGCGGCCGTTCATCGACGAGGCTAAGCGGCTGCGAG |
| | | CGCTGCACATGAAGGAGCACCCGGATTATAAATACCGGCCCCGGCGGAAA |
| | | ACCAAGACGCTCATGAAGAAGGATAAGTACACGCTGCCCGGCGGGCTGCT |
| | | GGCCCCCGGCGGCAATAGCATGGCGAGCGGGGTCGGGGTGGGCGCCGGCC |
| | | TGGGCGCGGGCGTGAACCAGCGCATGGACAGTTACGCGCACATGAACGGC |
| | | TGGAGCAACGGCAGCTACAGCATGATGCAGGACCAGCTGGGCTACCCGCA |
| | | GCACCCGGGCCTCAATGCGCACGGCGCAGCGCAGATGCAGCCCATGCACC |
| | | GCTACGACGTGAGCGCCCTGCAGTACAACTCCATGACCAGCTCGCAGACC |
| | | TACATGAACGGCTCGCCCACCTACAGCATGTCCTACTCGCAGCAGGGCAC |
| | | CCCTGGCATGGCTCTTGGCTCCATGGGTTCGGTGGTCAAGTCCGAGGCCA |
| | | GCTCCAGCCCCCCTGTGGTTACCTCTTCCTCCCACTCCAGGGCGCCCTGC |
| | | CAGGCCGGGGACCTCCGGGACATGATCAGCATGTATCTCCCCGGCGCCGA |
| | | GGTGCCGGAACCCGCCGCCCCCAGCAGACTTCACATGTCCCAGCACTACC |
| | | AGAGCGGCCCGGTGCCCGGCACGGCCATTAACGGCACACTGCCCCTCTCA |
| | | CACATGGCATGCGGCTCCGGCGAGGGCAGGGGAAGTCTTCTAACATGCGG |
| | | GGACGTGGAGGAAAATCCCGGCCCACTCGAGATGGCTGTCAGCGACGCGC |
| | | TGCTCCCATCTTTCTCCACGTTCGCGTCTGGCCCGGCGGGAAGGGAGAAG |
| | | ACACTGCGTCAAGCAGGTGCCCCGAATAACCGCTGGCGGGAGGAGCTCTC |
| | | CCACATGAAGCGACTTCCCCCAGTGCTTCCCGGCCGCCCCTATGACCTGG |
| | | CGGCGGCGACCGTGGCCACAGACCTGGAGAGCGGCGGAGCCGGTGCGGCT |
| | | TGCGGCGGTAGCAACCTGGCGCCCCTACCTCGGAGAGAGACCGAGGAGTT |
| | | CAACGATCTCCTGGACCTGGACTTTATTCTCTCCAATTCGCTGACCCATC |
| | | CTCCGGAGTCAGTGGCCGCCACCGTGTCCTCGTCAGCGTCAGCCTCCTCT |
| | | TCGTCGTCGCCGTCGAGCAGCGGCCCTGCCAGCGCGCCCTCCACCTGCAG |
| | | CTTCACCTATCCGATCCGGGCCGGGAACGACCCGGGCGTGGCGCCGGGCG |
| | | GCACGGGCGGAGGCCTCCTCTATGGCAGGGAGTCCGCTCCCCCTCCGACG |
| | | GCTCCCTTCAACCTGGCGGACATCAACGACGTGAGCCCCTCGGGCGGCTT |
| | | CGTGGCCGAGCTCCTGCGGCCAGAATTGGACCCGGTGTACATTCCGCCGC |
| | | AGCAGCCGCAGCCGCCAGGTGGCGGGCTGATGGGCAAGTTCGTGCTGAAG |
| | | GCGTCGCTGAGCGCCCCTGGCAGCGAGTACGGCAGCCCGTCGGTCATCAG |
| | | CGTCAGCAAAGGCAGCCCTGACGGCAGCCACCCGGTGGTGGTGGCGCCCT |
| | | ACAACGGCGGGCCGCCGCGCACGTGCCCCAAGATCAAGCAGGAGGCGGTC |
| | | TCTTCGTGCACCCACTTGGGCGCTGGACCCCCTCTCAGCAATGGCCACCG |
| | | GCCGGCTGCACACGACTTCCCCCTGGGGCGGCAGCTCCCCAGCAGGACTA |
| | | CCCCGACCCTGGGTCTTGAGGAAGTGCTGAGCAGCAGGGACTGTCACCCT |
| | | GCCCTGCCGCTTCCTCCCGGCTTCCATCCCCACCCGGGGCCCAATTACCC |
| | | ATCCTTCCTGCCCGATCAGATGCAGCCGCAAGTCCCGCCGCTCCATTACC |
| | | AAGAGCTCATGCCACCCGGTTCCTGCATGCCAGAGGAGCCCAAGCCAAAG |
| | | AGGGGAAGACGATCGTGGCCCCGGAAAAGGACCGCCACCCACACTTGTGA |
| | | TTACGCGGGCTGCGGCAAAACCTACACAAAGAGTTCCCATCTCAAGGCAC |
| | | ACCTGCGAACCCACACAGGTGAGAAACCTTACCACTGTGACTGGGACGGC |
| | | TGTGGATGGAAATTCGCCCGCTCAGATGAACTGACCAGGCACTACCGTAA |
| | | ACACACGGGGCACCGCCCGTTCCAGTGCCAAAAATGCGACCGAGCATTTT |
| | | CCAGGTCGGACCACCTCGCCTTACACATGAAGAGGCATTTTTAAATGACT |
| | | AGTGCGCGCAGCGGCCGACCATGGCCCAACTTGTTTATTGCAGCTTATAA |
| | | TGGTTACAAATAAAGCAATAGCATCACAAATTTCACAAATAAAGCATTTT |
| | | TTTCACTGCATTCTAGTTGTGGTTTGTCCAAACTCATCAATGTATCTTAT |
| | | CATGTCTGGATCTCGGTACCGGATCCAAATTCCCGATAAGGATCTTCCTA |
| | | GAGCATGGCTACGTAGATAAGTAGCATGGCGGGTTAATCATTAACTACAA |
| | | GGAACCCCTA |

### OTHER EMBODIMENTS

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Elements described with respect to one aspect or embodiment of the present disclosure are also contemplated with respect to other aspects or embodiments of the present disclosure. Moreover, recitation of claim elements in connection with a particular independent claim support recitation of such elements in connection with other independent claims. Throughout the disclosure and claims, where compositions or methods are described as having, including, or comprising specific elements, compositions that consist essentially of, consist of, or do not comprise the recited elements are likewise hereby disclosed. All references cited herein are hereby incorporated by reference. It is intended that the following claims define the scope of the invention and that methods and structures within the scope of these claims and their equivalents be covered thereby.

1-64. (canceled)

65. A method of treating a subject having a liver disease, comprising:
administering to the subject a pharmaceutical composition comprising one or more polynucleotides encoding octamer-binding transcription factor 4 (OCT4), sex determining region Y-box 2 (SOX2) and Krüppel-like factor 4 (KLF4),
wherein the pharmaceutical composition does not comprise a polynucleotide encoding a MYC protein.

66. The method of claim 65, wherein the method modifies the epigenetic state of one or more hepatocytes or stellate cells of the liver of the subject.

67. The method of claim 65, wherein the method does not reprogram a cell, tissue, or organ to a pluripotent state in the subject.

68. The method of claim 65, wherein the composition does not induce c-Myc expression and/or does not induce expression of Nanog in a cell, tissue, or organ of the subject.

69. The method of claim 65, wherein the one or more polynucleotides comprise DNA.

70. The method of claim 69, wherein a single polynucleotide encodes the OCT4, SOX2, and KLF4 and the one or more polynucleotides encoding OCT4, SOX2 and KLF4 are operably linked with one or more promoters.

71. The method of claim 70, wherein a single multicistronic polynucleotide encodes the OCT4, SOX2 and KLF4.

72. The method of claim 70, wherein the wherein the pharmaceutical composition comprises a viral vector comprising the one or more polynucleotides encoding OCT4, SOX2, and KLF4.

73. The method of claim 65, wherein the pharmaceutical composition comprises a lipid nanoparticle (LNP) comprising the one or more polynucleotides encoding OCT4, SOX2, and KLF4.

74. The method of claim 65, wherein the administration is systemic.

75. The method of claim 65, wherein the administration comprises an injection.

76. The method of claim 65, wherein the liver disease comprises liver steatosis, liver fibrosis, or liver cirrhosis.

77. The method of claim 65, wherein the liver disease comprises metabolic dysfunction-associated steatotic liver disease (MASLD) or metabolic dysfunction-associated steatohepatitis (MASH).

78. The method of claim 65, wherein the method decreases plasma ALT, decreases plasma AST, decreases plasma total bile acids, decreases plasma total cholesterol, decreases the percentage of hepatocytes with lipid droplets, decreases liver weight, improves NAFLD score of the subject by at least 1 point, and/or improves steatosis score of the subject by at least 1 point in the subject.

79. The method of claim 65, wherein the method does not cause a decrease in body weight of the subject and/or body weight of the subject does not decrease.

80. The method of claim 65, wherein the subject is administered, the subject has been administered, and/or the method comprises administering to the subject an obesity therapy.

81. The method of claim 65, wherein:
i) the OCT4 comprises an amino acid sequence having at least 90% identity to SEQ ID NO: 2;
ii) the SOX2 comprises an amino acid sequence having at least 90% identity to SEQ ID NO: 4; and/or
iii) the KLF4 comprises an amino acid sequence having at least 90% identity to SEQ ID NO: 6.

82. The method of claim 65, wherein:
i) the OCT4 comprises an amino acid sequence having at least 95% identity to SEQ ID NO: 2;
ii) the SOX2 comprises an amino acid sequence having at least 95% identity to SEQ ID NO: 4; and/or
iii) the KLF4 comprises an amino acid sequence having at least 95% identity to SEQ ID NO: 6.

83. The method of claim 65, wherein:
i) the OCT4 comprises the amino acid sequence of SEQ ID NO: 2;
ii) the SOX2 comprises the amino acid sequence of SEQ ID NO: 4; and/or
iii) the KLF4 comprises the amino acid sequence of SEQ ID NO: 6.

84. The method of claim 65, wherein the OCT4, SOX2, and KLF4 are the only transcription factors encoded by the pharmaceutical composition.
